SEQUENCE LISTING (39 sequences)

SEQ ID NO: 1 - protein, 121 aa, Griffithsia sp
SLTHRKFGGSGGSPFSGLSSIAVRSGSYLDXIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 2 - protein, 121 aa, synthetic construct
SLTHRKFGGSGGSPFSGLSSIAVRSGSYLDAIIIDGVHHGGSGGNLSPTFTFGSGEYISNMTIRSGDYIDNISFETNQGRRFGPYGGSGGSANTLSNVKVIQINGSAGDYLDSLDIYYEQY

SEQ ID NO: 3 - DNA, 366 bp, synthetic construct
  source 1..366: mol_type = other DNA
tcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatcagggcagacgctttggaccgtatggcggaagcggaggcagcgcaaatacactgtcaaatgttaaagtcattcagatcaatggcagcgcaggcgattatctggattcactggatatctattacgaacagtattaa

SEQ ID NO: 4 - protein, 269 aa, Kappaphycus alvarezil
MGRYTVQNQWGGSSAPWNDAGLWILGSRGNQNVMAIDVNSSDGGANLNGTMTYSGEGPIGFKGARRGDSNVYDVENQWGGSSAPWHAGGQFVIGSRSGQGVLAVNITSSDGGKTLTGTMTYEREGPIGFKGTQSGGDTYNVENQWGGSSAPWNKAGIWALGDRSGQAMIAMDVSSSDGGKTLEGTMQYKGEGPIGFRGKLSGANNYSVENQWGGSSAPWNKAGDWLIGDRHNQNITAVKVSSDNDGKNLDGTCTYEREGPIGFKGVATS

SEQ ID NO: 5 - protein, 144 aa, Musa sp.
MAGAIKVGTWGGNGGSEWDMGPAYRIDSVKINAGDIIDAIEITFTRYGLTETQHYGGTGGEPHEIAFEDGEYIMSMEGHVVDYTGLTIIGKLTLTTNRRTFGPFGAYEGTPFSIPVAEGKIAGFFGRAGSFIDAIGVYLMPNLE

SEQ ID NO: 6 - protein, 145 aa, Helianthus sp.
MANNYVEVGPWGGSGGANPWSIIPNGGRITRINVRSGAIVDAIYFGYTEGGTNYETAIFGGRNGSLSTIDIADDEEIIEINGKVATFENLNLVTQLTFVTNKQTYGPYGTNGGTDFSCPIAKGKVVGFFGRYGAYLDAIGVVLSP

SEQ ID NO: 7 - protein, 121 aa, Renouxia sp.
MTSVTLGGEGGRPFPSYNPRTIGGRSGAYIDQLTINGQVHGGQGGSPTSTFTFSDNEYIDSITVRHGLYIDSFSFTTSEGRRWGPYGGSGGSSTTIKNICVVGIAGRSGRFIDQLKIHYLY

SEQ ID NO: 8 - protein, 157 aa, Artocarpus sp.
DEQSGISQTVIVGPWGAKVSTSSNGKAFDDGAFTGIREINLSYNKETAIGDFQVVYDLNGSPYVGQNHKSFITGFTPVKISLDFPSEYIMEVSGYTGNVSGYVVVRSLTFKTNKKTYGPYGVTSGTPFNLPIENGLIVGFKGSIGYWLDYFSMYLSL

SEQ ID NO: 9 - protein, 101 aa, Nostoc ellipsosporum
LGKFSQTCYNSAIQGSVLTSTCERTNGGYNTSSIDLNSVIENVDGSLKWQPSNFIETCRNTQLAGSSELAAECKTRAQQFVSTKINLDDHIANIDGTLKYE

SEQ ID NO: 10 - protein, 114 aa, Microcystis sp.
MASYKVNIPAGPLWSNAEAQQVGPKIAAAHQGNFTGQWTTVVESAMSVVEVELQVENTGIHEFKTDVLAGPLWSNDEAQKLGPQIAASYGAEFTGQWRTIVEGVMSVIQIKYTF

SEQ ID NO: 11 - protein, 108 aa, Microcystis aeruginosa
MPNFSHTCSSINYDPDSTILSAECQARDGEWLPTELRLSDHIGNIDGELQFGDQNFQETCQDCHLEFGDGEQSVWLVCTCQTMDGEWKSTQILLDSQIDNNDSQLEIG

SEQ ID NO: 12 - protein, 156 aa, Homo sapiens
AERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 13 - protein, 134 aa, Kappaphycus alvarezil
APAADTTASPALGSQVSAQFASVTIRNAQTGRLLDSNYNGNVYTLPANGGNYQRWTGPGDGTVRNAQTGRCLDSNYDGAVYTLPCNGGSYQKWLFYSNGYIQNVETGRVLDSNYNGNVYTLPANGGNYQKWYTG

SEQ ID NO: 14 - protein, 269 aa, Eucheuma sp.
MGRYTVQNQWGGSSAPWNDAGLCILGSRGNQNVIAVDVTSSDGGANLGGTMTYSGEGPIGFKGARRGESNVYDVENQWGGSSAPWHAGGQFVIGSRSGQGVTALSVTSSDGGKTLTGTMTYEREGPIGFKGTQSGGDTYNVENQWGGSSAPWNKAGIWALGDRNGQAMIAMDVSSSDGGQTLEGTMQYKGEGPIGFRGKLSGANNYAVENQWGGSSAPWNKAGDWLIGDRYNQNITAVKVSSDNDGKNLDGTCTYEREGPIGFKGVATS

SEQ ID NO: 15 - protein, 268 aa, Meristotheca sp.
MGRYTVQNQWGGSSAPWNDAGLYVLGSRANQNVVAIEVSSNDGGANLTGTMTYSGEGPIGFKGARRGESNVYEVKNQWGGSSAPWHDGGDFVIGSRSGQGVVGLDIKSSDNGKTLTGTMTYEREGPIGFKGTQSGGDTYNVENQWGGSSAPWNKAGVWALGDRSSQAMIKLEVSSSDGGSTLEGTMQYKGEGPIGFRAKLASANNYSVENQWGGSSAPWNKAGDWLIGDRYNQNITAVNVSSSDDGKNLEGTCTYAREGPIGFKGSAV

SEQ ID NO: 16 - protein, 133 aa, Oscillatoria sp.
MALYNVENQWGGSSAPWNEGGQWEIGSRSDQNVVAINVESGDDGQTLNGTMTYAGEGPIGFRATLLGNNSYEVENQWGGDSAPWHSGGNWILGSRENQNVVAINVESGDDGQTLNGTMTYAGEGPIGFKGTLT

SEQ ID NO: 17 - protein, 268 aa, Eucheuma sp.
GRYTVQNQWGGSSAPWNDAGLWILGSRGNQNVMAVDVNSSDGGANLNGTMTYSGEGPIGFKGARRGESNVYDVENQWGGSSAPWHAGGQFVIGSRSGQGVLAVNITSSDGGKTLTGTMTYEREGPIGFKGTQSGGDTYNVENQWGGSSAPWNKAGIWALGDRSGQAMIAMDVSSSDGGKTLEGTMQYKGEGPIGFRGKLSGANNYSVENQWGGSSAPWNAAGDWLIGDRHNQNITAVKVSSDNDGKNLDGTCTYEREGPIGFKGVATS

SEQ ID NO: 18 - protein, 133 aa, Herpetosiphon aurantiacus
MSVYWVENQWGGDSAPWHPGGTWVLGARDNQNVVAINISSADNGQTFTGTMTYINEGPIGFRATRTSANNYAVENQWGGDSAPWHPGGHWIIGTRDNQNPVNLDVDSRDGGQTLNGTMVYAGEGPIGFRGKLQ

SEQ ID NO: 19 - protein, 149 aa, Roseofilum sp.
MSDYAVKNQWGGSGAPWHNAGTWVLGSRQNQLVVSIDIESNDNGKTFSGTITYEGEGPIGFKAEQISGNKYNTQNQWGGNDAPWHEGGTWVIGQRVDQRVVSLKVHSDDKGQTLVGNNTYDGEGPVGFIGVLVLVGAASQKTNQVATHA

SEQ ID NO: 20 - protein, 133 aa, Pseudomonas baetica
MSKYVVANQWGGSSAPWHPGGDWVLGARDNQNVVAIEIKSGDDGKTFTGTMTYAGEGPIGFKAQRTGQNQYNVENQWGGNDAPWHPGGKWVIGGRDNQNVVALSVTSNDGGKNLSGTNTYANEGPIGFRGQIE

SEQ ID NO: 21 - protein, 263 aa, Verrucomicrobiaceae sp.
NHYKVLNQWGEESAPWKDGGSWVMGSRSEQNIVALEVKSDDGGETLIGTVTYEGEGPIGFKGESGEPHTYYVKNEFGGAWHEGGAWYIGDRDNQEVVALSLKSEDGGRTLEGTMTYSGEGAIRVRAALFNAYEVENRWGDAAEPWRAGGHWTLGGRLEQKAVQFSIKSDDDGQTLTGTMTYAGEGSVDFRAKRVKQNTYKVENHWGDAAEPWHEAGTWKLGGRDDQNVVGLEVKSENEGKDFSGTVTYKGEGPIDFRARIFVP

SEQ ID NO: 22 - protein, 267 aa, Melittangium boletus
MSIYNVQNQWGGSSAAWNQGGLWAIGGRSNQGVIALKAKSEDGGKTLTGTMTYSGEGEIGFRATLSGTNTYTVQNQWGGSSAPWQEGGTWILGSRKNQGVVAIDLTSNDGGKTLAGTMTYAGEGPIGFKGELRSGGAYTVQNQWGGASAPWQQGGAWLIGARDGQKVTAIKATSTDGGKTLTGSMTYSGEGAIAFKATQDGENTYTVQNQWGGSAAPWQPGGIWLIGARQSQGVVSLDVTSTDGGKTLAGTMTYAGEGPIGFRGTLN

SEQ ID NO: 23 - protein, 273 aa, Rhodocyclaceae sp.
MAKISKQENGLYHVQNQWGGNSAPWHDGGMWVIGARGAQKVITLQVHSNDGGRSLFGIMSYAGEGPIGFRATLTQTNTYVVENNWGDSPWQPGGTWLLGARNGQNVVSIDIRSEDNGGTYTGAITYSGEGPIGFKAGGVDGGVYVAENQWGGNQAPWYPGGLWVIGCRGTQYVVGVDYTSADGGRSINGTMTYMEEGPIGLKGAVALADTYITENQWGGSSAPWHPGGEWVLGCRGNQLVVGLKVSSPDSGKTLTGTTTYAGEGPIGFRASLR

SEQ ID NO: 24 - protein, 131 aa, Longispora albida
ADTTASPALGSQVSAQFASVTIRNAQTGRLLDSNYNGNVYTLPANGGNYQRWTGPGDGTVRNAQTGRCLDSNYDGAVYTLPCNGGSYQKWLFYSNGYIQNVETGRVLDSNYNGNVYTLPANGGNYQKWYTG

SEQ ID NO: 25 - protein, 135 aa, Mus musculus
MACGLVASNLNLKPGECLKVRGEVASDAKSFVLNLGKDSNNLCLHFNPRFNAHGDANTIVCNTKEDGTWGTEHREPAFPFQPGSITEVCITFDQADLTIKLPDGHEFKFPNRLNMEAINYMAADGDFKIKCVAFE

SEQ ID NO: 26 - protein, 135 aa, Desmodus rotundus
MTSNLVAHNVNLRPGRSIRVQGEVAADPKSFALNLGTDSNNLCLHFNPRFKIFGDVNKTVYNSRKAGVWEKELRDPVFPFQPGSVIEVCITFDVAELTIKLPDGHTFKFPNRHNLETINYLETNGDLKIKSVTFE

SEQ ID NO: 27 - protein, 133 aa, Scleropages formosus
MSLTLKNMSIKAGKELKITGVPKPDALSFSVNIGHGEDNIALHFNPRFNYQGDNNVIVFNSRHGGSWSEERRENHFPFEQGEEFKVTISFNNEEFRVKLPDGNMLYFPNRFGDDKYMYIQVEGDARVTGFKLK

SEQ ID NO: 28 - protein, 142 aa, Callorhinchus milii
MKLVLEMFNVNMKPGDTLKIKGVIEPDAERFAVNLGTDPSRYALHFNPRFDDEVDGCVIVCNNKEEGEWSTEQRDEDFPLKKGESFKISITFQGDVFEIKLPEDRVLEFPNRSCAEILTYLVIEGQVRVKGLKFDYLPEVAL

SEQ ID NO: 29 - protein, 268 aa, Trichodesmium sp.
MTLYKVENQWGGSSAPWHPGGDWVIGGRSNQNVVAIDVKSDDGGETLNGTMTYIGEGPIGFRATRTGKNNNYTVENQWGGSSAPWHPGGDWVIGGRSDQNVVALNIKSDDDGKTLNGTMTYIGEGPIGFKGTLMNLYKVENQWGGSSAPWHPGGDWVIGGRSNQNVVAIDVKSDDGGETLNGTMTYIGEGPIGFRATRTGKNNNYTVENQWGGSSAPWHPGGDWVIGGRSDQNVVALNIKSDDDGKTLNGTMTYVGEGPIGFKGTLVG

SEQ ID NO: 30 - protein, 276 aa, Burkholderia ubonensis
MAKTSKGFNNLQHVQNQWGGSSAPWHEGGMWVLGCRSGQNVVALNIESRDGGRTFNGTMTYAGEGPIGFKATLTQSNTYVVENQWGGSSAPWHPGGTWMIGCRVNQNVVALSIESGDGGATLAGSMTYAGEGPIGFKSQQADGGVYAVENQWGGSSAPWHNGGVWVIGARDQAVVALNVSSKDSGKTLEGNMTYAGEGHIGFKGNSVAGNNYAVENQWGGSSAAWHPGGVYLLGCRAGQNVVEVNITSGDNGNTFHGSMTYSGEGPIGFRATELPQ

SEQ ID NO: 31 - protein, 274 aa, Aquimarina sp.
MAIFNVQNQWGGNSAPWHAGGTWVLGSRDNQNVVEIDIKSGDGGRTFTGTMTYAGEGPIGFKAKQISGNNYTVENQWGGDSAPWHPAGNWIIGGREGQNVIALKVNSVDDSANLEGTMIYAGEGPIGFRGLETDGSSYSVENQWGGHTAPWHDGGTFVLGARKDQNPVAYDINSTDGGKTFTGTMTYQGEGPIGFRATKIGANNYAAENQWGGDSAPWHPGGNLIIGSRTNQNVVNLKINSTDEGKTFTGEMTYQGEGPIGFKGVLSDNVLAKA

SEQ ID NO: 32 - protein, 136 aa, Microcystis aeruginosa
MSLLYQVENQWGGDNAPWNNGGTWVMGCRPSQNIIAIHIKSDDGGKTFHGNMTYAGEGPIGFRATLSGGNNYFVQNQWGGDNAPWNNGGTWVIGGRSQQNVVELNLQSDDGGNHLKGTMTYNGEGPIGFKGTMIPS

SEQ ID NO: 33 - protein, 267 aa, Corallococcus sp.
MTAYVVQNQWGGSGAPWNPGGLWVIGSRPGQGVVAINVTSSDGGKTLTGTMTYNGEGPIGFRGTLSDGNNYTVENQWGGSSAPWQPGGVWVLGARKGQHIVALNVTSSDGGNTLLGTTTYNGEGPIGFRSEQSNGGAYTVENQWGGASAPWQPGGVWVLGARKGQNVVAVSVTSSDGGKTLTGTMTYNGEGPIGFRGTLSDGNNYTVENQWGGSSAPWQPGGLWIIGARQNQNVVALNFTSPDGGKALTGTTTYNGEGPIGFRGKLN

SEQ ID NO: 34 - protein, 268 aa, Agardhiella subulata
MARYTVQNQWGGSSAPWNDAGLYILGNRANQNVMAIDVTSSDGGAVLTGTMTYSGEGPIGFKGTRRGDSTVYEVENQWGGSSAPWRPGGDFVIGSRAGQGVVGLDVSSSDDGKTLTGTMTYEREGPIGFKGTQSGGDSYNVENQWGGSSAPWNKAGIWALGDRNGQGMIGLDVTSPDGGKTLEGTMQYKNEGPIGFRGKLSGANNYSVENQWGGSSAPWNKAGNWLIGDRHNQNIVAVKVSSTDNGKNLEGTCTYAGEGPVGFKGVAN

SEQ ID NO: 35 - protein, 273 aa, Sphingomonas sp.
MTVYVIQNQWGGISAPWHEGGVFNIGNRADQRPVALKIQSGDGGHSFSGTMTYQGEGPIGVRATLVTTNCYRVENQWGGSNAPWHDAGLFLLGARNGQNAVAFDLRSDDQGDTLTGTMTYQGEGPIGVKAAVSDGVAYNAQNQWGGDQAPWHPGGQFILGCRPDQPVVAIDVSSADSGKTLAGTITYKGEGPIGFRGTLIMANTYSVVNQWGGNDKPWHPGGTWVLGCRTNQGVVAIKAAGDGVEVDGTMTYQGEGPIGLELQRSSQQALADA

SEQ ID NO: 36 - protein, 274 aa, Proteobacteria sp.
MSNTNSYKVQVQWGGSAAPWHQDGIWVVGGRDNQSAVALNIISHNGGQTFTGTMQYEGEGPLGFRAQQESGNSYNTWVQWGGSSAPWHENGIWVIGGRDGQRGVEVNISSGDGGQTFLGNIIYAGEGPIGFKAEQDFKAGFNSYDTEVQWGGSAEPWHANGVWIIGGRDKQHAVAVDINSGDGGKSFTGSMTYAGEGPLGFQATLKGDNEYDTWVQWGGSYAPWHQNGVWVIGGRDKQNAVALKIDSKDGGETFTGTMTYDGEGPIGFKAKNTD

SEQ ID NO: 37 - protein, 273 aa, Sinobacterium sp.
MALYQVENQWGGNSAPWHEGGKWIIGSRDNQNPIAVDLQSFDGGKTLKGTMSYEGEGVISVRATRHGCNNYRVENQWGGSDAQWHDAGQWILGFRADQSIIALSVKSTDGGNTLSGTTTYESEGAIGFNASRAPGEAFTVVNQWGGSQEPWREGGTWVLGSRLDQAVIAIDVQSNDNGKTLTGTMTYANEGAIGFKASLIGANNYKVQNQWGGSSAPWQPAGIWVIGYRNNQPVTALSLLTDDQGETLSGEMTYLNEGSIAISASVVGETEPA

SEQ ID NO: 38 - protein, 276 aa, Aquimarina sp.
MAIYQVQNQWGGNSAPWHAGGTWVLGGRDNQNVVAIDIKSGDGGRTFSGTMTYEGEGPIGFKAIQIAGNNYSVENQWGGASAPWHPGGNWIIGGRNGQNVIELNVTAESGSANLEGTMKYAGEGPIGFKGQETVGSSYSIENQWGGASAPWHPGGTFVLGARENQNPVAYDIQSTDGGKTFTGTMTYAGEGPIGFRAIQTAGNNYAAENQWGGASAPWHPGGNLVIGARVNQNVVQLKINSNDNGETFSGEMTYLGEGPIGVKAVLSSRVLSGATS

SEQ ID NO: 39 - protein, 149 aa, Musa troglodytarum
MHACVQAGAVIKVGKWGADGGSHWDTGPADHIKGIKINAAGVVDSITFMYVIGETADETPRYGGPGGKHNQIKFFDEEYLNAISGCIGEYDGTPCISQLTFTTNMGTYGPYGDGGGTPFNLPVDEGKIAGFYGRAGTYLIAIGAYLKPN

(19) United States
(12) Patent Application Publication
(10) Pub. No.: US 2025/0197910 A1
(43) Pub. Date: Jun. 19, 2025

(54) COMPOSITIONS AND METHODS FOR INDUSTRIAL SCALE PRODUCTION AND RECOVERY OF LECTINS

(71) Applicants: DANISCO US INC., Palo Alto, CA (US); DANISCO (CHINA) HOLDING CO., LTD., Shanghai (CN)

(72) Inventors: Cristina Bongiorni, Fremont, CA (US); Gopal K. Chotani, Cupertino, CA (US); David A. Estell, San Mateo, CA (US); Jeffrey Veach Miller, Menlo Park, CA (US); Matthew Reboli, Sunnyvale, CA (US); Chao Zhu, Mountain View, CA (US); Meng Hong HENG, Belmont, CA (US); Chunli Lei, Shanghai (CN); Qiang Zhao, Shanghai (CN); Zhongmei Tang, Shanghai (CN)

(21) Appl. No.: 18/849,402

(22) PCT Filed: Mar. 23, 2023
(86) PCT No.: PCT/US2023/064847

§ 371 Date: Sep. 20, 2024

Related U.S. Application Data
(60) Provisional application No. 63/323,256, filed on Mar. 24, 2022.

(51) Int. Cl. C12P 21/00; C07K 14/405; C12N 15/75

(52) U.S. Cl. CPC C12P 21/005; C07K 14/405; C12N 15/75; C12N 2800/101

(57) ABSTRACT

Certain aspects of the instant disclosure are related to the industrial scale production and recovery of lectin proteins. Certain aspects are related to recombinant Gram-positive bacterial cells producing heterologous lectin proteins. Certain other aspects are related to compositions and methods for recovering and/or purifying one or more lectins and enhanced purity lectin preparations thereof.

### CROSS REFERENCE TO REPLATED APPLICATIONS

This application claims benefit to U.S. Provisional Application No. 63/323,256, filed Mar. 24, 2022, which is hereby incorporated by reference in its entirety.

### FIELD

The present disclosure is generally related to the fields of microbial cells, molecular biology, fermentation, protein production, protein recovery, protein purification, protein preparations, and the like. Certain aspects of the disclosure are related to the industrial scale production and recovery of lectin proteins, recombinant Gram-positive bacterial cells producing heterologous lectins, compositions, and methods for recovering and/or purifying one or more lectins, enhanced purity lectin preparations thereof and the like.

### REFERENCE TO A SEQUENCE LISTING

The contents of the electronic submission of the text file Sequence Listing, named “NB41857-WO-PCT_SequenceListing.txt” was created on Mar. 21, 2023, and is 41 KB in size, which is hereby incorporated by reference in its entirety.

### BACKGROUND OF THE DISCLOSURE

Lectins are generally defined as carbohydrate binding proteins that can recognize and bind simple or complex carbohydrates in a reversible and highly specific manner, while displaying no catalytic activity (Lagarda-Diaz et al., 2017). Lectin proteins were originally named hemagglutinins, due to their ability to agglutinate red blood cells (and other cells). More recently, lectins such as the red algae (Griffithsia sp.) griffithsin (GRFT) protein, the red algae (Kappaphycus alvarezii) KAA-2 protein, the concanavalin A (ConA) protein from jack-beans, the jacalin protein from jackfruit (A. heterophyllus), the cyanovirin-N (CV-N) protein from cyanobacteria (N. ellipsosporum) and the like, have been evaluated for their anti-viral activities (Whitley et al., 2013).

For example, PCT Publications WO2005/118627 and WO2007/064844, describe methods for isolating the native griffithsin (GRFT) lectin from red algae (Griffithsia sp.), cloning the wild-type (grft) gene thereof, generating recombinant polynucleotides thereof, fermenting and producing the same in E. coli host cells, followed by isolating the recombinant His-tagged GRFT protein from the E. coli host, and characterizing its anti-viral activity. PCT Publication No. WO2010/01424 generally describes methods of inhibiting a hepatitis C viral infection of a host comprising administering to the host an effective amount of a glycosylation resistant GRFT (variant) protein (or a polypeptide conjugate thereof) in combination with another anti-viral protein. For example, as described in this publication, the inventors noted that the anti-viral protein combination of scytovirin (SVN) and griffithsin (GRFT) have (nanomolar) activity against the Hepatitis C virus (HCV).

U.S. Patent Publication No. US20110263485 describes methods of inhibiting a human immunodeficiency virus (HIV) infection of a host comprising administering to the host an effective amount of a gp120 Griffithsin and a peptide selected from a gp41-binding protein, a CCR5-binding protein, a gp120-binding protein, or another griffithsin, which combinations are potent inhibitors to HIV infection. PCT Publication No. WO2016/130628 discloses variant griffithsin proteins having mutations that change the isoelectric point of the GRFT protein, which are reported to alter its solubility in various pH ranges allowing for improved product release. PCT Publication No. WO2019/108656 generally describes microbicidal compositions comprising an endosperm extract and an anti-HIV lectin, an anti-HIV antibody, or antigen binding antibody fragment thereof.

The recombinant production of GRFT in tobacco plants (Nicotiana benthamiana) has been described by O'Keefe et al. (2009), wherein the GRFT accumulates to a level of about 1 gram of recombinant GRFT per kilogram of Nicotiana benthamiana leaf material, when expressed via an infectious tobacco mosaic virus (TMV) based vector. For example, as contemplated in the O'Keefe et al. publication, despite the promise that biologics such as griffithsin have as HIV prophylactics, their practical application as topical microbicides is hampered by high production costs, wherein it is unlikely that any manufacturing system reliant on growth in sterile conditions can be competitive with the price of a male condom, which is necessary if the product is to be available for use by those at risk for sexual transmission of HIV.

Hirayama et al. (2016) have described the elucidated primary structure of KAA-2 lectin using peptide mapping and complementary DNA (cDNA) cloning and prepared its active recombinants using an E. coli expression system. Gengenbach et al. (2019) have described the transient expression of the mistletoe lectin named “viscumin” (Viscum album) in intact Nicotiana benthamiana plants, and purification of the recombinant viscumin from crude plant extracts by affinity chromatography, wherein the performance and economics of tobacco plant-based process was compared to the corresponding process based on E. coli expression. As summarized by Gengenbach et al., the E. coli process has a low recovery, requires extensive dilution and is complex, whereas the plant-based process included only half the number of steps. According to a direct cost comparison between the two processes performed in the Gengenbach et al. study, the plant expression system was 50% less expensive, in comparison with the native host V. album or the heterologous expression in E. coli host cells.

Petrova et al. (2016) have described a probiotic Lactobacillus rhamnosus strain (GR-1), documented to survive implantation onto the vaginal epithelium and interfere with urogenital pathogens. As set forth in this publication, a genomic region encoding a protein with homology to lectin-like proteins was identified (i.e., llp1 gene encoding the lectin-like protein 1 (Llp1)), wherein phenotypic analysis of the knock-out mutant strain (GR-1_Δllp1) of the L. rhamnosus (GR-1) parent strain revealed a two-fold decreased adhesion to the vaginal and ectocervical epithelial cell lines compared to wild-type.

More recently, Petrova et al. (2018) described probiotic L. rhamnosus strains expressing (HIV-inhibiting lectins) actinohivin (AH) or griffithsin (GRFT) for in situ delivery. As generally summarized in this publication, L. rhamnosus strains were not able to produce intracellular or extracellular AH, postulating that the observed results might be that the AH is toxic during export out of the cell wall of L. rhamnosus strains, possibly by binding to essential glycosylated cell wall molecules, such as peptidoglycan, exopolysaccharides, or glycosylated proteins of the Sec pathway. Petrova et al. (2018) further describe L. rhamnosus strains constructed for the extracellular expression of GRFT, wherein the obtained results “suggest that L. rhamnosus strains can synthesize GRFT, but does not to secrete it out of the cells under the tested conditions”. For example, as set forth in this publication, for recombinant strains CMPG10731, CMPG10734, CMPG10767 and CMPG10768, bands corresponding to GRFT were detected in the cell wall fractions, suggesting possible trapping of the recombinant protein in the cell wall.

Based on the foregoing, it is apparent that there remain ongoing and unmet needs in the art for improved host organisms capable of producing heterologous lectins for various anti-microbial uses, as well as ongoing and unmet needs for novel methods and compositions enabling the cost-effective industrial scale production, recovery and/or purification of lectins.

### SUMMARY OF THE INVENTION

As described and exemplified hereinafter, the instant disclosure provides, inter alia, novel recombinant (modified) Gram-positive bacterial cells (strains) expressing heterologous lectin proteins, polynucleotides (e.g., vectors, expression cassettes) comprising nucleic acids encoding heterologous lectins, methods, and compositions for producing heterologous lectins in recombinant Gram-positive host cells, methods, and compositions for recovering and/or purifying lectins, and the like.

Certain one or more embodiments or aspects of the disclosure are therefore related to methods for expressing/producing heterologous lectins in Gram-positive bacterial (host) cells. In certain embodiments, the disclosure provides methods for producing heterologous lectins in a Gram-positive bacterial cell by introducing into the cell an expression cassette encoding the lectin, wherein the cassette comprises an upstream (5′) promoter sequence operably linked to a downstream (3′) open reading frame (ORF) encoding the lectin, and fermenting the cell under suitable conditions for the production of the lectin. In certain embodiments of the methods, at least two cassettes encoding the lectin are introduced into the cell, wherein the cassettes comprise an upstream promoter sequence operably linked to a downstream ORF encoding the lectin. In certain other embodiments of the methods, the introduced cassette is integrated into the genome of the cell. In certain other embodiments of the methods, the at least two introduced cassettes are integrated into the genome of the cell. In other one or more embodiments of the methods, the at least two introduced cassettes comprise the same ORF encoding the same lectin, or comprise different open reading frames (ORFs) encoding different lectins.

Certain other one or more embodiments or aspects of the disclosure are related to methods for producing heterologous lectins in a Gram-positive bacterial cell comprising introducing into the cell an expression cassette encoding the lectin, wherein the cassette comprises an upstream (5′) promoter sequence operably linked to a downstream nucleic acid encoding a signal (secretion) sequence operably linked to a downstream (3′) open reading frame (ORF) encoding the lectin, and fermenting the cell under suitable conditions for the production of the lectin. In certain embodiments of the methods, the introduced cassette is integrated into the genome of the cell. In yet other embodiments of the methods, the cell comprises at least two introduced cassettes encoding the lectin, wherein the cassettes comprise an upstream promoter sequence operably linked to a downstream nucleic acid encoding a signal sequence operably linked to a downstream ORF encoding the lectin. In other one or more embodiments of the methods, the at least two introduced cassettes are integrated into the genome of the cell. In certain other embodiments of the methods, the at least two introduced cassettes comprise the same ORF encoding the same lectin, or comprise different ORFs encoding different lectins.

Certain other one or more embodiments or aspects of the disclosure are related to methods for producing heterologous lectins in a Gram-positive bacterial cell comprising introducing into the cell a first and a second expression cassette encoding the lectin, wherein the first cassette comprises an upstream promoter sequence operably linked to a downstream open reading frame (ORF) encoding the lectin and the second cassette comprises an upstream promoter sequence operably linked to a downstream nucleic acid encoding a signal (secretion) sequence operably linked to a downstream ORF encoding the lectin, and fermenting the cell under suitable conditions for the production of the lectin. In certain embodiments of the methods, the first introduced cassette, or the second introduced cassette, is integrated into the genome of the cell. In other embodiments of the methods, the first and second introduced cassettes are integrated into the genome of the cell. In yet other embodiments of the methods, the at least two introduced cassettes comprise the same ORF encoding the same lectin, or comprise different ORFs encoding different lectins.

In any of the preceding embodiments of the methods, the lectin is expressed intracellularly and/or the lectin is expressed and secreted extracellularly.

In yet other embodiments of the methods, the ORF encodes a native lectin, or a variant lectin, derived from a cyanobacterial cell, a eukaryotic cell, or a bacterial cell. In certain other embodiments of the methods, a native or variant lectin is derived from a eukaryotic cell selected from the group consisting of plant cells, fungal cells, insect cells, and animal cells.

In other one or more embodiments of the methods, the ORF encodes a lectin selected from the group consisting of a native griffithsin (GRFT) lectin (or a variant GRFT lectin derived therefrom), a native scytovirin (SVN) lectin (or a variant SVN lectin derived therefrom), a native cyanovirin-N (CVN) lectin (or a variant CVN lectin derived therefrom), a native K. alvarezii KAA-1 lectin (or a variant KAA-1 lectin derived therefrom), a native K. alvarezii KAA-2 lectin (or a variant KAA-2 lectin derived therefrom), a native Microcystis viridis (MVL) lectin (or a variant MVL lectin derived therefrom), a native Boodlea coacta agglutinin (BCA) lectin (or a variant BCA lectin derived therefrom), a native Artocarpus heterophyllus (Jacalin) lectin (or a variant Jacalin lectin derived therefrom), a native Musa acuminata (Banana) lectin (or a variant Banana lectin derived therefrom), a native Aaptos papilleta (Sponge) lectin (or a variant Sponge lectin derived therefrom), a native Abrus precatorius (Jequirty bean) lectin (or a variant Jequirty bean lectin derived therefrom), a native Aegapodium podagraria (Ground elder) lectin (or a variant Ground elder lectin derived therefrom), an Agaricus bisporus (Common mushroom) lectin (or a variant Common mushroom lectin derived therefrom), a native Albizzia julibrissin (Mimosa tree seed) lectin (or a variant Mimosa tree seed lectin derived therefrom), a native Allomyrina dichotoma (Japanese beetle) lectin (or a variant Japanese beetle lectin derived therefrom), a native Aloe arborescens (Aloe plant) lectin (or a variant Aloe plant lectin derived therefrom), a native Amphicarpaea bracteata (Hog peanut) lectin (or a variant Hog peanut lectin derived therefrom), a native Anguilla (Eel) lectin (or a variant Eel lectin derived therefrom), a native Aplysia depilans (Mollusca) lectin (or a variant Mollusca lectin derived therefrom), a native Arachis hypogaea (Peanut) lectin (or a variant Peanut lectin derived therefrom), a native Bauhinia purpurea (Camel's foot tree) lectin (or a variant Camel's foot tree lectin derived therefrom), a native Bryonia diocia (White bryony) lectin (or a variant White bryony lectin derived therefrom), a native Caragana Arborescens (Siberian pea tree) lectin (or a variant Siberian pea tree lectin derived therefrom), a native Carcinoscorpius rotundacauda (Horseshoe crab) lectin (or a variant Horseshoe crab lectin derived therefrom), a native Microcystis aeruginosa (cyanobacterium) microvirin (MVN) lectin (or a variant MVN lectin derived therefrom), a native Eucheuma serra (red algae) ESA-2 lectin (or a variant ESA-2 lectin derived therefrom), a native Musa acuminate (Banana) BanLec lectin (or a variant BanLec lectin derived therefrom), a native Aspidistra elatior AEL lectin (or a variant AEL lectin derived therefrom), a native Chaetopterus variopedatus (Marine worm) CVL lectin (or a variant CVL lectin derived therefrom), a Vicia faba (Fava bean) lectin (or a variant Fava bean lectin derived therefrom), a native Lens culinaris (lentil) (or a variant lentil lectin derived therefrom), a native Pisum sativum (pea) lectin (or a variant pea lectin derived therefrom), a native jacalin-like lectin (or variant jacalin-like lectin derived therefrom), including but not limited to a jacalin-like lectin set forth in SEQ ID NO: 5, SEQ ID NO: 6, SEQ ID NO: 7, SEQ ID NO: 8 and SEQ ID NO: 39, a native CVN-like lectin (or variant CVN-like lectin derived therefrom), including but not limited to a CVN-like lectin set forth in SEQ ID NO: 10 and SEQ ID NO: 11, a native OAA-like lectin (or variant OAA-like lectin derived therefrom), including but not limited to an OAA-like lectin set forth in one of SEQ ID NO: 14-SEQ ID NO: 23 or SEQ ID NO: 29-SEQ ID NO: 38, a native galectin-1-like lectin (or variant galectin-1-like lectin derived therefrom), including but not limited to a galectin-1-like lectin set forth in SEQ ID NO: 25-SEQ ID NO: 28, and a native ricin-like lectin (or variant ricin-like lectin derived therefrom), including but not limited to a ricin-like lectin set forth in SEQ ID NO: 24.

In certain other embodiments of the methods, a Gram-positive bacterial cell is selected from a member of the Firmicutes phylum.

Certain other one or more embodiments or aspects of the disclosure are related to methods for recovering (and optionally purifying) lectins produced by Gram-positive bacterial cells. Thus, certain one or more embodiments of the disclosure provide methods for recovering heterologous lectins expressed and retained intracellularly and/or provides methods for recovering heterologous lectins expressed and secreted extracellularly.

Certain embodiments are therefore directed to methods for recovering an intracellular lectin comprising fermenting a recombinant cell expressing an intracellular lectin and lysing cells at end of the fermentation to obtain a lysed cell broth, heat treating the lysed broth at pH between 1.5 and 8.5, then cooling broth and harvesting the cooled broth, subjecting the harvested cooled broth to a clarification process, and subjecting the clarified broth to a concentration process, wherein the lectin is recovered in the clarified concentrated broth. In certain aspects of the methods, a pH between 1.5 and 8.5 is about pH 1.5, about pH 2.0, about pH 2.5, about pH 3.0, about pH 3.5, about pH 4.0, about pH 4.5, about pH 5.0, about pH 5.5, about pH 6.0, about pH 6.5, about pH 7.0, about pH 7.5, about pH 8.5 to about pH 9.0.

Certain other embodiments are therefore related to methods for recovering a secreted lectin comprising fermenting a recombinant cell expressing and secreting a lectin, and harvesting the broth, subjecting the harvested broth to a clarification process, and subjecting the clarified broth to a concentration process, wherein the lectin is recovered in the clarified concentrated broth.

Certain other embodiments are related to methods for enhancing the purity of a lectin concentrate comprising obtaining a lectin concentrate recovered according to any of the preceding methods, (a) adjusting the pH of concentrate to between 1.5 to 8.5, (b) incubating the concentrate of step (a) for a sufficient amount of time at a temperature between about 5° C. and 55° C., and (c) centrifuging the concentrate and collecting the supernatant, or filtering the concentrate and collecting the filtrate, wherein the collected supernatant, or the collected filtrate, comprises a substantially purified lectin relative to the purity of the lectin concentrate obtained in step (a). In certain embodiments or aspects of step (a), a pH between 1.5 and 8.5 is about pH 1.5, about pH 2.0, about pH 2.5, about pH 3.0, about pH 3.5, about pH 4.0, about pH 4.5, about pH 5.0, about pH 5.5, about pH 6.0, about pH 6.5, about pH 7.0, about pH 7.5, about pH 8.5 to about pH 9.0.

Certain other embodiments provide methods for enhancing the purity of a lectin concentrate comprising obtaining a lectin concentrate recovered according to any of the preceding methods, (a) adjusting the pH of the concentrate to about pH 2, incubating the concentrate at pH 2 for a sufficient amount of time at a temperature between about 55° C. and 65° C., and (b) centrifuging the incubated concentrate and collecting the supernatant, or filtering the concentrate and collecting the filtrate, wherein the collected supernatant, or the collected filtrate, comprises a substantially purified lectin relative to the purity of the lectin concentrate obtained in step (a). In certain embodiments or aspects of step (a), a temperature between about 55° C.-65° C. is about 54.5° C. about 55° C., about 56° C., about 57° C., about 58° C., about 59° C., about 60° C., about 61° C., about 62° C. about 63° C., about 64° C., about 65° C. to about 65.5° C.

In other one or more embodiments, the disclosure provides methods for the crystallization of a lectin comprising (a) obtaining a lectin concentrate recovered according to one of the preceding methods and (b) adding a salt or a mixture of salts, comprising sodium, calcium, ammonium, sulfate, or chloride ions to the concentrate, adjusting the pH of the salted concentrate to about pH 2 to 5, and mixing and incubating the concentrate between about 5° C. to 50° C. for an appropriate amount of time, wherein the lectin crystallizes from the concentrate after mixing and incubating for an appropriate amount of time in step (b). In certain aspects, the pH of salted concentrate is adjusted to about pH 2, about pH 2.5, about pH 3, about pH 3.5, about pH 4, about pH 4.5, or about pH 5.

In certain other one or more embodiments, the disclosure provides methods for the crystallization of a lectin comprising (a) obtaining a lectin concentrate recovered according to one of the preceding methods and (b) adding a salt or a mixture of salts at about 0.5% to about 10%, comprising sodium, calcium, ammonium, sulfate, or chloride ions to the concentrate, adjusting the pH of the salted concentrate to about pH 2 to 5, and mixing and incubating the concentrate between about 5° C. to 50° C. for an appropriate amount of time, wherein the lectin crystallizes from the concentrate after mixing and incubating for an appropriate amount of time in step (b). In certain related embodiments of the method, the pH is about 2.5 to about 3.5, the salt is about 1% to about 5% sodium sulfate, and the incubating temperature is about 5° C. to about 25° C. In other embodiments of the method, the pH is about 2.8 to about 3.2, the salt is about 1.8 to about 2.5% sodium sulfate, and the incubating temperature is about 15° C. to about 25° C.

One or more other embodiments of the disclosure are related to methods for enhancing the purity of a lectin (protein) preparation comprising (a) obtaining a lectin crystal slurry according to one of the preceding methods, optionally washing the crystal slurry with water and mixing, and centrifuging the slurry for sufficient amount of time, decanting, and collecting the supernatant following centrifugation, adding appropriate pH buffers to maintain pH of about 4.8 to about 8.6 with mixing at room temperature for a sufficient amount of time, and (b) filtering supernatant and collecting filtered supernatant, wherein the collected supernatant comprises a high purity lectin preparation.

In certain other one or more embodiments, the disclosure provides methods for recovering (and optionally purifying) lectins produced by other recombinant cells expressing native or heterologous lectins, such as recombinant tobacco cells/plants expressing heterologous lectins, recombinant E. coli cells expressing heterologous lectins, and the like. Thus, certain embodiments or aspects of the disclosure provide methods for recovering a secreted lectin from a fermentation broth in which a recombinant cell has been fermented for the expression of a lectin. In certain embodiments, such methods comprise (a) obtaining and harvesting a whole fermentation broth comprising a secreted lectin, (b) subjecting the harvested broth to a clarification process, and (c) subjecting the clarified broth to a concentration process, wherein the lectin is recovered in the clarified concentrated broth. Certain other one or more embodiments of the disclosure are related to methods for recovering an intracellular lectin generally comprising (a) obtaining and lysing a whole fermentation comprising a recombinant cell which has been fermented for the expression of the lectin, heat treating the lysed broth at pH between about 1.5 and about 8.5, then cooling broth and harvesting the cooled broth. (b) subjecting the harvested cooled broth to a clarification process, and (c) subjecting the clarified broth to a concentration process, wherein the lectin is recovered in the clarified concentrated broth. In related embodiments, the disclosure provides methods for enhancing the purity of a lectin concentrate comprising (a) obtaining a lectin concentrate recovered according to a preceding method of this paragraph, adjusting the pH of concentrate to between about 1.5 to about 8.5. (b) incubating the concentrate of step (a) for a sufficient amount of time at a temperature between about 5° C. and about 55° C., and (c) centrifuging the concentrate and collecting the supernatant, or filtering the concentrate and collecting the filtrate, wherein the collected supernatant, or the collected filtrate, comprises a substantially purified lectin relative to the purity of the lectin concentrate obtained in step (a). In certain other related embodiments, the disclosure provides methods for enhancing the purity of a lectin concentrate comprising (a) obtaining a lectin concentrate recovered according to a preceding method of this paragraph, adjusting the pH of the concentrate to about pH 2, incubating the concentrate at about pH 2 for a sufficient amount of time at a temperature between about 55° C. and about 65° C., and (b) filtering the incubated concentrate and collecting the supernatant, or filtering the concentrate and collecting the filtrate, wherein the collected supernatant, or the collected filtrate, comprises a substantially purified lectin relative to the purity of the lectin concentrate obtained in step (a).

|0029| In other one or more related embodiments, the disclosure provides methods for the crystallization of a lectin comprising (a) obtaining a lectin concentrate recovered according to a method set forth above, (b) adding a salt or a mixture of salts, comprising sodium, calcium, ammonium, sulfate, or chloride ions to the concentrate, adjusting the pH of the salted concentrate to about 2 to about 5, and mixing and incubating the concentrate between about 5° C. to about 50° C. for an appropriate amount of time, wherein the lectin crystallizes from the concentrate after mixing and incubating for an appropriate amount of time in step (b).

In certain other one or more related embodiments, the disclosure provides methods for the crystallization of a lectin comprising (a) obtaining a lectin concentrate recovered according to the according to a method set forth above, (b) adding a salt or a mixture of salts at about 0.5% to about 10% to the concentrate, the salt or mixture of salts comprising sodium, calcium, ammonium, sulfate, or chloride ions, adjusting the pH of the salted concentrate to pH of about 2 to about 5, and mixing and incubating the concentrate between about 5° C. to about 50° C. for an appropriate amount of time, wherein the lectin crystallizes from the concentrate after mixing and incubating for an appropriate amount of time in step (b). In certain aspects, the pH in the preceding method is about 2.5 to about 3.5, the salt is about 1% to about 5% sodium sulfate, and the incubating temperature is about 5° C. to about 25° C. In other embodiments or aspects, the pH in the preceding methods is about 2.8 to about 3.2, the salt is about 1.8% to about 2.5% sodium sulfate, and the incubating temperature is about 15° C. to about 25° C.

Thus, certain other one or more embodiments provide methods for enhancing the purity of a lectin preparation comprising (a) obtaining a lectin crystal slurry according to a method set forth above, optionally washing the crystal slurry with water and mixing, and centrifuging the slurry for sufficient amount of time, decanting, and collecting the supernatant following centrifugation, adding appropriate pH buffers to maintain pH of about 4.8 to about 8.6 with mixing at room temperature for a sufficient amount of time, and (b) filtering supernatant and collecting filtered supernatant, wherein the collected supernatant comprises a high purity lectin preparation.

Certain other embodiments are therefore directed to high purity lectin preparations produced and obtained according to one or more methods of the disclosure.

In particular embodiments, the disclosure provides methods for producing and recovering a high purity griffithsin (GRFT) protein preparation comprising (a) constructing a recombinant Gram-positive bacterial cell expressing a polynucleotide encoding the GRFT protein, (b) fermenting the cell under suitable conditions for the production of the GRFT protein, lysing cells at end of the fermentation to obtain a lysed cell broth, and treating the lysed broth by holding broth for about 1 to about 4 hours at a pH of about 4.8 to about 5.2 and a temperature of about 50° C. to about 80° C., (c) clarifying the broth of step (b) by a filtration or microfiltration process, and concentrating the clarified broth by an ultrafiltration process. (d) performing a crystallization process on the concentrated broth of step (c), the crystallization process comprising adding about 2% sodium sulfate to the concentrate, adjusting pH to about 3 and incubating at about 22° C. for a sufficient amount of time, (e) centrifuging the incubated concentrate of step (d), decanting supernatant to harvest the GRFT crystal, dissolving the GRFT crystal in about 100 mM sodium acetate buffer at about pH 4.5 to about 5.5 to obtain an aqueous GRFT protein preparation, and centrifuging the GRFT preparation to remove any insoluble impurities therein and collecting the supernatant or filtering the GRFT preparation to remove any insoluble impurities therein and collecting the filtrate, wherein the supernatant collected in step (e), or filtrate collected in step (e), comprises a high purity GRFT protein preparation.

In yet other embodiments the disclosure relates to methods for recovering a high purity GRFT preparation comprising (a) obtaining a whole fermentation broth comprising recombinant cells expressing the GRFT protein, lysing cells in the cell broth, and treating the lysed broth by holding broth for about 1 to 4 hours at a pH of about 4.8 to 5.2 and a temperature of about 50° C. to 80° C., (b) clarifying the broth of step (b) by a filtration or microfiltration process, and concentrating the clarified broth by an ultrafiltration process, (c) performing a crystallization process on the concentrated broth of step (b), the crystallization process comprising adding about 2% sodium sulfate to the concentrate adjusting pH to about 3 and incubating at about 22° C. for a sufficient amount of time, and (d) centrifuging the incubated concentrate of step (c), decanting supernatant to harvest the GRFT crystal, dissolving the GRFT crystal in about 100 mM sodium acetate buffer at about pH 4.5 to about 5.5 to obtain an aqueous GRFT protein preparation, and centrifuging the GRFT preparation to remove any insoluble impurities therein, and collecting the supernatant, or filtering the GRFT preparation to remove any insoluble impurities therein and collecting the filtrate, wherein the supernatant collected in step (d), or filtrate collected in step (d), comprises a high purity GRFT protein preparation.

In certain embodiments of the methods, the high purity GRFT preparation comprises a native GRFT protein, or a variant GRFT protein preparation. In certain other embodiments of the methods, the high purity GRFT preparation is at least 2.0 times higher in purity than the recovered GRFT concentrate, as determined via the GRFT concentration measured at A₂₈₀ nm. In certain other embodiments of the methods, the GRFT is the major band, or the only band of about 12.7 kDa in the high purity GRFT preparation when visualized by SDS-PAGE. In one or more other embodiments, the high purity GRFT comprises hemagglutination activity when assayed/screened against one or more animal red blood cells (erythrocytes).

In certain other embodiments of the methods, recombinant cells of the disclosure are rendered deficient in the production of one or more endogenous genes encoding one or more highly expressed background (native) proteins, including, but not limited to amylases, xylanases, pullulanases, phytases, pectate lyases, glucanases, mannosidases, lipases, esterases and the like.

Certain other embodiments of the disclosure are therefore related to one or more recombinant Gram-positive bacterial cells (strains) expressing heterologous lectins. In certain embodiments, the disclosure is related to a recombinant Gram-positive bacterial cell expressing a polynucleotide encoding a heterologous lectin. In certain embodiments, the recombinant cell comprises an introduced polynucleotide cassette encoding the lectin, wherein the cassette comprises an upstream promoter operably linked to a downstream nucleic acid encoding the lectin, or wherein the cassette comprises an upstream promoter operably linked to a downstream nucleic acid encoding a signal sequence operably linked to a downstream nucleic acid encoding the lectin. In other embodiments, the recombinant cell comprises at least two introduced cassettes encoding the lectin, wherein the at least two introduced cassettes comprise an upstream promoter operably linked to a downstream nucleic acid encoding the lectin. In yet other embodiments, the recombinant cell comprises at least two introduced cassettes encoding the lectin, wherein the introduced cassettes comprise an upstream promoter operably linked to a downstream nucleic acid encoding a signal sequence operably linked to a downstream nucleic acid encoding the lectin. In certain other embodiments, the recombinant cell comprises at least two introduced cassettes encoding the lectin, wherein one introduced cassette comprises an upstream promoter operably linked to a downstream nucleic acid encoding the lectin and the second introduced cassette comprises an upstream promoter operably linked to a downstream nucleic acid encoding a signal sequence operably linked to a downstream nucleic acid encoding the lectin.

In certain embodiments of the recombinant cells, the lectin is expressed intracellularly. In certain other embodiments of the recombinant cells, the lectin is expressed and secreted extracellularly. In yet other embodiments of the recombinant cells, the lectin is expressed intracellularly, and the lectin expressed and secreted extracellularly.

In certain other embodiments of the recombinant cells, the cassette is integrated into the genome of the cell. In other embodiments of the recombinant cells, at least one of the at least two introduced cassettes are integrated into the genome of the cell. In yet other embodiments of the recombinant cells, the at least two introduced cassettes are integrated into the genome of the cell.

In other embodiments of the recombinant cells, the at least two introduced cassettes encode the same lectin, or encode different lectins.

In other embodiments of the recombinant cells, the Gram-positive bacterial cell is selected from a member of the Firmicutes phylum. In other embodiments, the Gram-positive bacterial cell is selected from a Bacillaceae family member. In certain other embodiments, the Gram-positive bacterial cell is selected from the group consisting of Alkalibacillus sp. cells, Amphibacillus sp. cells, Anoxybacillus sp. cells, Bacillus sp. cells, Caldalkalibacillus sp. cells, Cerasilbacillus sp. cells, Exiguobacterium sp. cells, Filobacillus sp. cells, Geobacillus sp. cells, Gracilibacillus sp. cells, Halobacillus sp. cells, Halolactibacillus sp. cells, Jeotgalibacillus sp. cells, Lentibacillus sp. cells, Marinibacillus sp. cells, Oceanobacillus sp. cells, Ornithinibacillus sp. cells, Paraliobacillus sp. cells, Paucisalibacillus sp. cells, Pontibacillus sp. cells, Pontibacillus sp. cells, Saccharococcus sp. cells. Salibacillus sp. cells, Salinibacillus sp. cells, Tenuibacillus sp. cells, Thalassobacillus sp. cells, Ureibacillus sp. cells, and Virgibacillus sp. cells.

In other one or more embodiments of the recombinant cells, the heterologous lectin is derived from a bacterial cell, a eukaryotic cell, or a cyanobacterial cell. In another embodiment of the recombinant cells, the heterologous lectin is derived from a eukaryotic cell is selected from the group consisting of plant cells, fungal cells, insect cells, and animal cells. In certain other one or more embodiments of the recombinant cells, the heterologous lectin is selected from the group consisting of a native griffithsin (GRFT) lectin (or a variant GRFT lectin derived therefrom), a native scytovirin (SVN) lectin (or a variant SVN lectin derived therefrom), a native cyanovirin-N (CVN) lectin (or a variant CVN lectin derived therefrom), a native K. alvarezii KAA-1 lectin (or a variant KAA-1 lectin derived therefrom), a native K. alvarezii KAA-2 lectin (or a variant KAA-2 lectin derived therefrom), a native Microcystis viridis (MVL) lectin (or a variant MVL lectin derived therefrom), a native Boodlea coacta agglutinin (BCA) lectin (or a variant BCA lectin derived therefrom), a native Artocarpus heterophyllus (Jacalin) lectin (or a variant Jacalin lectin derived therefrom), a native Musa acuminata (Banana) lectin (or a variant Banana lectin derived therefrom), a native Aaptos papilleta (Sponge) lectin (or a variant Sponge lectin derived therefrom), a native Abrus precatorius (Jequirty bean) lectin (or a variant Jequirty bean lectin derived therefrom), a native Aegapodium podagraria (Ground elder) lectin (or a variant Ground elder lectin derived therefrom), an Agaricus bisporus (Common mushroom) lectin (or a variant Common mushroom lectin derived therefrom), a native Albizzia julibrissin (Mimosa tree seed) lectin (or a variant Mimosa tree seed lectin derived therefrom), a native Allomyrina dichotoma (Japanese beetle) lectin (or a variant Japanese beetle lectin derived therefrom), a native Aloe arborescens (Aloe plant) lectin (or a variant Aloe plant lectin derived therefrom), a native Amphicarpaea bracteata (Hog peanut) lectin (or a variant Hog peanut lectin derived therefrom), a native Anguilla (Eel) lectin (or a variant Eel lectin derived therefrom), a native Aplysia depilans (Mollusca) lectin (or a variant Mollusca lectin derived therefrom), a native Arachis hypogaea (Peanut) lectin (or a variant Peanut lectin derived therefrom), a native Bauhinia purpurea (Camel's foot tree) lectin (or a variant Camel's foot tree lectin derived therefrom), a native Bryonia diocia (White bryony) lectin (or a variant White bryony lectin derived therefrom), a native Caragana Arborescens (Siberian pea tree) lectin (or a variant Siberian pea tree lectin derived therefrom), a native Carcinoscorpius rotundacauda (Horseshoe crab) lectin (or a variant Horseshoe crab lectin derived therefrom), a native Microcystis aeruginosa (cyanobacterium) microvirin (MVN) lectin (or a variant MVN lectin derived therefrom), a native Eucheuma serra (red algae) ESA-2 lectin (or a variant ESA-2 lectin derived therefrom), a native Musa acuminate (Banana) BanLec lectin (or a variant BanLec lectin derived therefrom), a native Aspidistra elatior AEL lectin (or a variant AEL lectin derived therefrom), a native Chaetopterus variopedatus (Marine worm) CVL lectin (or a variant CVL lectin derived therefrom), a Vicia faba (Fava bean) lectin (or a variant Fava bean lectin derived therefrom), a native Lens culinaris (lentil) (or a variant lentil lectin derived therefrom), a native Pisum sativum (pea) lectin (or a variant pea lectin derived therefrom), a native jacalin-like lectin (or variant jacalin-like lectin derived therefrom), including but not limited to a jacalin-like lectin set forth in SEQ ID NO: 5, SEQ ID NO: 6, SEQ ID NO: 7, SEQ ID NO: 8 and SEQ ID NO: 39, a native CVN-like lectin (or variant CVN-like lectin derived therefrom), including but not limited to a CVN-like lectin set forth in SEQ ID NO: 10 and SEQ ID NO: 11, a native OAA-like lectin (or variant OAA-like lectin derived therefrom), including but not limited to an OAA-like lectin set forth in one of SEQ ID NO: 14-SEQ ID NO: 23 or SEQ ID NO: 29-SEQ ID NO: 38, a native galectin-1-like lectin (or variant galectin-1-like lectin derived therefrom), including but not limited to a galectin-1-like lectin set forth in SEQ ID NO: 25-SEQ ID NO: 28, and a native ricin-like lectin (or variant ricin-like lectin derived therefrom), including but not limited to a ricin-like lectin set forth in SEQ ID NO: 24.

In certain other embodiments, recombinant cells of the disclosure are rendered deficient in the production of one or more endogenous genes encoding one or more highly expressed background (native) proteins, including, but not limited to amylases, xylanases, pullulanases, phytases, pectate lyases, glucanases, mannosidases, lipases, esterases and the like.

### BRIEF DESCRIPTION OF DRAWINGS

FIG. 1 shows the mature amino acid sequences of exemplary lectins GRFT (SEQ ID NO: 1), Q-GRFT (SEQ ID NO: 2) and KAA-2 (SEQ ID NO: 4). More particularly, the native griffithsin (GRFT) protein comprises 121 amino acid residues, wherein the amino acid “X” at position 31 of the native GRFT is an unknown, non-naturally occurring amino acid residue (FIG. 1A, SEQ ID NO: 1). A variant of the native griffithsin protein is set forth in FIG. 1B (Q-GRFT; SEQ ID NO: 2). For example, the variant Q-GRFT comprises 121 amino acid residues, wherein the non-naturally occurring residue X at position 31 of the native GRFT (FIG. 1A; SEQ ID NO: 1) has been substituted with an alanine (A) at position 31 of Q-GRFT (FIG. 1B; SEQ ID NO: 2) and the methionine (M) residue at position 78 of the native GRFT (FIG. 1A; SEQ ID NO: 1) has been substituted with a glutamine (Q) at position 78 of Q-GRFT (FIG. 1B; SEQ ID NO: 2). The native KAA-2 lectin protein comprises 269 amino acid residues (FIG. 1C; SEQ ID NO: 4).

FIG. 2 presents the mature amino acid sequences of exemplary jacalin-like lectins (FIG. 2A-2E).

FIG. 3 presents the mature amino acid sequences of exemplary CVN-like lectins (FIG. 3A-3B).

FIG. 4 presents the mature amino acid sequences of exemplary OAA-like lectins (FIG. 4A-4T).

FIG. 5 presents the mature amino acid sequences of exemplary ricin-like and galectin-like lectins (FIG. 5A-5E).

FIG. 6 presents schematic diagrams of exemplary lectin polynucleotide expression cassettes of the disclosure. More particularly, as shown in FIG. 6, expression cassettes may be constructed for intracellular expression (FIG. 6A, FIG. 6B), or extracellular expression/secretion (FIG. 6C. FIG. 6D) of the lectin protein in a Gram positive (host) cell. As shown in FIG. 6, the promoter sequence (abbreviated, “pro”), the signal sequence (abbreviated, “ss”), and the optional terminator sequence (abbreviated, “term”) of the expression cassettes are generally selected so as to be functional in the desired Gram-positive host. As shown in FIG. 6C and FIG. 6D, the DNA sequence (lectin) encoding the mature lectin protein is positioned downstream (3′) and is operably linked to the (5′) nucleic acid sequence (ss) encoding the secretion signal sequence. For enhanced expression of the cassette in a specific Gram-positive (host) cell, the DNA sequence (lectin) encoding the mature lectin protein may be codon optimized using techniques and methods known to those skilled in the art.

FIG. 7 shows an SDS-PAGE gel of broth supernatants obtained from B. subtilis strains CB447 (secreted Q-GRFT) and CB476 (intracellular Q-GRFT), which strains were evaluated in 10 L bioreactors. More specifically, as presented in FIG. 7, ten (10) μL of 10-fold diluted samples (lanes 1-6), along with the See Blue Plus 2 molecular weight standard (labeled, “kDa”) and the T4 lysozyme protein standard (McLab), followed by staining and detaining of the gel using standard molecular biology procedures. As shown in FIG. 7, lanes 1, 2 and 3 are the broth supernatants from strain CB447 at eighteen (18) hours, twenty-four (24) hours and thirty (30) hours of fermentation, respectively, and lanes 4, 5 and 6 are the broth supernatants of strain CB476 at eighteen (18) hours, twenty-four (24) hours and thirty (30) hours of fermentation, respectively, wherein the Q-GRFT protein appears as a single band with a molecular weight of 12.7 kDa.

FIG. 8 presents an SDS-PAGE gel of Q-GRFT protein preparations (FIG. 8) described in Example 11. As presented in FIG. 8, the lane labeled “MW” is a molecular weight ladder, lane 1 is crystal slurry at 47 hours, lane 2 is no wash crystal slurry supernatant, lanes 3, 4 and 5 are no wash crystal pellet plus buffer, lanes 6, 7 and 8 are no wash crystal pellet plus buffer, filtered, lane 9 is 1× wash crystal slurry supernatant, lanes 10, 11 and 12 are washed crystal pellet plus buffer, lanes 13, 14 and 15 are washed crystal pellet plus buffer, filtered, as described in Example 11. For example, the buffer in lanes 3, 6, 10, 13 is 100 mM Tris (pH 8.6), the buffer in lanes 4, 7, 11, 14 is 100 mM Bis-Tris (pH 6.5) and the buffer in lanes 5, 8, 12, 15 is 100 mM sodium acetate (pH 5.5).

FIG. 9 shows a chromatogram indicating the total protein composition of the purified lectin (Q-GRFT) described in Example 14. As presented in FIG. 9, the Q-GRFT purity was about 90% of the total protein composition.

FIG. 10 presents a schematic diagram of plasmid p3JM for expressing lectin gene (ORF) coding sequences. As shown in FIG. 10, p3JM comprises an upstream (5′) B. subtilis aprE promoter region operably linked to a downstream (3′) nucleic acid coding a lectin protein of interest operably linked to a downstream terminator (Tlat) sequence. In addition, plasmid p3JM (FIG. 10) includes a β-lactamase gene coding sequence (Bla) and chloramphenicol acetyltransferase coding gene (CAT).

FIG. 11 shows an SDS-PAGE analysis of purified jacalin-like, CVN-like, OAA-like, Ricin-like, and galectin-1-like lectins expressed in Gram-positive bacterial cells. More particularly, FIG. 11 shows SDS-PAGE results of various lectins with amino sequences (SEQ ID NO, e.g., see FIG. 1-5) expressed in the supernatant of B. subtilis strain CBS12. These lectins expressed embody a variety of different molecular weights, structural folds, and species of origin.

FIG. 12 shows the hemagglutination activity of lectins on different erythrocytes. More particularly, FIG. 124 presents the hemagglutination activity of different doses of lectins on 1% mouse erythrocytes, showing lectin hemagglutination is dose dependent. FIG. 12B shows the hemagglutination activity of different lectins (100 μg/ml) on 1% animal erythrocytes. The lane marked PB indicates phosphate buffer used as a control.

FIG. 13 shows hemagglutination activity of jacalin-like, CVN-like, OAA-like and galectin-like lectins produced in Gram-positive bacterial cells of the disclosure. The lane marked PB indicates phosphate buffer used as a control. The various lectins annotated with sequence identification numbers (SEQ ID Nos.) at the top of the gel were tested against different animal erythrocytes. Hemagglutination activity suggests that the lectins produced are functionally active.

### BRIEF DESCRIPTION OF THE BIOLOGICAL SEQUENCES

SEQ ID NO: 1 is the amino acid sequence of the native, mature griffithsin (GRFT) protein isolated from Griffithsia sp.

SEQ ID NO: 2 is the amino acid sequence of a variant griffithsin (Q-GRFT) protein having a methionine (M) to glutamine (Q) substitution at amino acid position 78 (M78Q).

SEQ ID NO: 3 is a DNA sequence encoding the Q-GRFT protein (SEQ ID NO: 2), which DNA sequence has been codon optimized for expression in B. subtilis cells.

SEQ ID NO: 4 is the amino acid sequence of the native, high-mannose binding lectin KAA 2 isolated from Kappaphycus alvarezii.

SEQ ID NO: 5 is the amino acid sequence of a Musa acuminate (Banlec) lectin.

SEQ ID NO: 6 is the amino acid sequence of a Helianthus annuus lectin.

SEQ ID NO: 7 is the amino acid sequence of a Renouxia sp. lectin.

SEQ ID NO: 8 is the amino acid sequence of a Artocarpus integrifolia (Jacalin) lectin.

SEQ ID NO: 9 is the amino acid sequence of a Nostoc ellipsosporum (CV-N) lectin.

SEQ ID NO: 10 is the amino acid sequence of a Microcystis viridis (MVL) lectin.

SEQ ID NO: 11 is the amino acid sequence of a Microcystis aeruginosa PCC7806 (MVN) lectin.

SEQ ID NO: 12 is the amino acid sequence of a Homo sapiens (DCSIGN) lectin.

SEQ ID NO: 13 is the amino acid sequence of a Kappaphycus alvarezil (KAA-1) lectin.

SEQ ID NO: 14 is the amino acid sequence of a Eucheuma denticulatum (EDA2) lectin.

SEQ ID NO: 15 is the amino acid sequence of a Meristotheca papulosa (MPA-2) lectin.

SEQ ID NO: 16 is the amino acid sequence of a Oscillatoria agardhii NIES-204 (OAA) lectin.

SEQ ID NO: 17 is the amino acid sequence of a Eucheuma serra (ESA-2) lectin.

SEQ ID NO: 18 is the amino acid sequence of a Herpetosiphon aurantiacus DSM 785 lectin.

SEQ ID NO: 19 is the amino acid sequence of a Roseofilum reptotaenium AO1-C lectin.

SEQ ID NO: 20 is the amino acid sequence of a Pseudomonas baetica lectin.

SEQ ID NO: 21 is the amino acid sequence of a Verrucomicrobiaceae bacterium lectin.

SEQ ID NO: 22 is the amino acid sequence of a Melittangium boletus lectin.

SEQ ID NO: 23 is the amino acid sequence of a Rhodocyclaceae bacterium lectin.

SEQ ID NO: 24 is the amino acid sequence of a Longispora albida (Actinohivin) lectin.

SEQ ID NO: 25 is the amino acid sequence of a Mus musculus (Galectin-1) lectin.

SEQ ID NO: 26 is the amino acid sequence of a Desmodus rotundus (Galectin-1) lectin.

SEQ ID NO: 27 is the amino acid sequence of a Scleropages formosus (Galectin-1) lectin.

SEQ ID NO: 28 is the amino acid sequence of a Callorhinchus milii lectin.

SEQ ID NO: 29 is the amino acid sequence of a Trichodesmium sp. ALOHA_ZT_67 lectin.

SEQ ID NO: 30 is the amino acid sequence of a Burkholderia ubonensis lectin.

SEQ ID NO: 31 is the amino acid sequence of a Aquimarina longa lectin.

SEQ ID NO: 32 is the amino acid sequence of a Microcystis aeruginosa lectin.

SEQ ID NO: 33 is the amino acid sequence of a Corallococcus sp. Z5C101001 lectin.

SEQ ID NO: 34 is the amino acid sequence of a Agardhiella subulata lectin.

SEQ ID NO: 35 is the amino acid sequence of a Sphingomonas sp. TDK1 lectin.

SEQ ID NO: 36 is the amino acid sequence of a Proteobacteria bacterium lectin.

SEQ ID NO: 37 is the amino acid sequence of a Sinobacterium caligoides lectin.

SEQ ID NO: 38 is the amino acid sequence of a Aquimarina sp. TRLI lectin.

SEQ ID NO: 39 is the amino acid sequence of a Musa troglodytarum lectin.

### DETAILED DESCRIPTION

### I. Overview

As briefly set forth above, and described hereinafter, certain embodiments of the disclosure provide, inter alia, novel recombinant Gram-positive bacterial cells expressing heterologous lectin proteins, wherein the lectin proteins can be the same lectin or combinations of different lectin proteins, recombinant polynucleotides (e.g., vectors, expression cassettes) encoding heterologous lectins for introducing (e.g., transforming) into Gram-positive host cells for the expression of the heterologous lectins, fermentation broths comprising lectin proteins (and lectin preparations obtained therefrom), lectin proteins recovered from a fermentation broth (and lectin preparations obtained therefrom), purified lectin preparations, and the like. Certain embodiments of the disclosure therefore provide novel methods for the recovery and/or purification of lectins derived from any naturally occurring lectin sources (e.g., plant lectins, algal lectins, cyanobacterial lectins, etc.). Certain other embodiments of the disclosure therefore provide novel methods for the recovery and/or purification of lectins obtained from recombinant cells expressing one or more lectins (e.g., recombinant Gram-negative bacterial cells, recombinant Gram-positive bacterial cells, recombinant plant cells (e.g., tobacco) and the like).

### II. DEFINITIONS

In view of the recombinant cells expressing heterologous lectins, polynucleotides comprising nucleic acids encoding heterologous lectins, one or more recombinant lectins (e.g., native lectins and/or functional variants thereof) encoded by such recombinant host cells, fermentation processes thereof for producing such recombinant lectins, recovery and/or purification processes thereof and the like, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely”, “only”, “excluding”, “not including” and the like, in connection with the recitation of claim elements, or use of a “negative” limitation or proviso thereof. An example of a proviso used herein, in certain embodiments, a “recombinant lectin protein” produced and/or recovered and/or purified according to the instant disclosure is not a “His-tagged lectin”.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the terms “recombinant” or “non-natural” refer to an organism, microorganism, cell, nucleic acid molecule, vector and the like that has at least one engineered genetic alteration, or has been modified by the introduction of a heterologous nucleic acid molecule; or refer to a cell (e.g., a Gram-positive cell) that has been altered such that the expression of a heterologous nucleic acid molecule or an endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions, or other functional alteration of a cell's genetic material. For example, recombinant cells may express genes or other nucleic acid molecules (e.g., polynucleotide constructs) that are not found in identical or homologous form within a native (wild-type) cell or may provide an altered expression pattern of endogenous genes, such as being over-expressed, under-expressed, minimally expressed, or not expressed at all. “Recombination”, “recombining” or generating a “recombined” nucleic acid is generally the assembly of two or more nucleic acid fragments wherein the assembly gives rise to a chimeric DNA sequence that would not otherwise be found in the genome.

The term “derived” encompasses the terms “originated”, “obtained.” “obtainable,” and “created,” and generally indicates that one specified material or composition finds its origin in another specified material or composition or has features that can be described with reference to another specified material or composition. For example, recombinant Gram-positive bacterial cells of the disclosure may be derived/obtained from any known Gram-positive bacterial strains (e.g., B. subtilis 168 strain, etc.). Native lectin proteins (and/or functional lectin variants thereof) and the DNA sequences encoding the same, may be derived/obtained from known lectin proteins and/or functional variants thereof.

As used herein, an “endogenous gene” refers to a gene in its natural location in the genome of an organism.

As used herein, a “heterologous” gene, a “non-endogenous” gene, or a “foreign” gene refer to a gene (or gene coding sequence (CDS) or (ORF)) not normally found in the host organism, but that is introduced into the host organism by gene transfer. The term “foreign” gene(s) comprise native genes (or ORF's) inserted into a non-native organism and/or chimeric genes inserted into a native or non-native organism.

As used herein, a “heterologous control sequence”, refers to a gene expression control sequence (e.g., promoters, enhancers, terminators, etc.) which does not function in nature to regulate (control) the expression of the gene of interest. Generally, heterologous nucleic acids are not endogenous (native) to the cell, or a part of the genome in which they are present, and have been added to the cell, by infection, transfection, transduction, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase during translocation.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”. “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter. “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located (3′) downstream to a promoter sequence. Promoters may be derived in their entirety from a native gene or be composed of different elements derived from different promoters found in nature, or even comprise synthetic nucleic acid segments.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

Thus, a nucleic acid is “operably linked” when it is placed into a functional relationship with another nucleic acid sequence. For example, DNA encoding a secretory leader (e.g., secretory signal sequence) is operably linked to DNA for a polypeptide if it is expressed as a pre-protein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in a desired Gram-positive host cell. For example, in certain embodiments, the present disclosure is directed to a polynucleotide comprising an upstream (5′) promoter (or 5′ promoter region, or tandem 5′ promoters and the like) functional in a Gram-positive cell, wherein the promoter region is operably linked to a nucleic acid sequence (e.g., an ORF) encoding a lectin protein.

As used herein, “suitable regulatory sequences” refer to nucleotide sequences located upstream (5′ non-coding sequences), within, or downstream (3′ non-coding sequences) of a coding sequence, and which influence the transcription. RNA processing or stability, or translation of the associated coding sequence. Regulatory sequences may include promoters, transcription leader sequences, RNA processing site, effector binding site and stem-loop structures.

As used herein, “a functional gene” is a gene capable of being used by cellular components to produce an active gene product, typically a protein. Functional genes are the antithesis of mutagenized genes, which are modified such that they cannot be used by cellular components to produce an active gene product or have a reduced ability to be used by cellular components to produce an active gene product.

As used herein, a “functional protein” is a protein that possesses an activity, such as an enzymatic activity, a binding activity, a surface-active property, or the like, and which has not been mutagenized, truncated, or otherwise modified to abolish or reduce that activity.

As used herein, the terms “modification” and “genetic modification” are used interchangeably and include, but are not limited to: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein (such that a functional protein is not made), internal deletions, insertions disrupting the coding sequence, mutations removing the operable link between a native promoter required for transcription and the open reading frame, and the like.

As used herein, the term “introducing”, as used in phrases such as “introducing into a bacterial cell” or “introducing into a bacterial cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein “an incoming sequence” refers to a DNA sequence that is introduced into the Gram-positive host cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” or “homology arm” refers to a nucleic acid sequence, which is homologous to a sequence in the Gram-positive bacterial cell's chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Gram-positive bacterial cell's chromosome a DNA construct is integrated and directs what part of the Gram-positive bacterial cell's chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R}. Other markers useful in accordance with the invention include, but are not limited to auxotrophic markers, such as serine, lysine, tryptophan; and detection markers, such as β-galactosidase.

As defined herein, a host cell “genome”, a Gram-positive bacterial (host) cell “genome”, a Bacillus sp. (host) cell “genome” and the like, include chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. In some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophages, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously) or can integrate into a chromosome of a host organism.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental Bacillus sp. (host) cell is modified (e.g., transformed) by introducing therein one or more “targeting vectors”.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

As used herein, a “host cell” refers to a cell that has the capacity to act as a host or expression vehicle for a newly introduced DNA sequence.

In certain aspects, a host cell of the disclosure is a Gram-positive bacterial cell/strain. As appreciated by one skilled in the art, many Gram-positive host strains are generally recognized as safe (GRAS) per US FDA guidelines, and as such, Gram-positive host cells are particularly useful protein production hosts relative to Gram-negative hosts (e.g., E. coli expression systems), which require additional costly processing steps to remove endotoxins (e.g., LPS).

As used herein, B. subtilis strains herein named “CB447”, “CB476”, “CB460”, “CB462”, “CB488”, “CBS6”, “CZ438”, “CBS6” and “CBS12” were constructed for the expression of the mature Q-GRFT variant (SEQ ID NO: 2). More particularly, as described in the Examples section, B. subtilis strains CB447, CB476, CB460 and CZ438 were constructed for extracellular secretion of the mature Q-GRFT variant, B. subtills strain CB476 was constructed for intracellular expression of the mature Q-GRFT variant. B. subtillis strains CBS6 and CBS12 were constructed for intracellular or secreted expression of the mature Q-GRFT variant, and B. subtillis strain CB488 was constructed for both intracellular and secreted expression of the mature Q-GRFT variant. In certain embodiments, recombinant Gram-positive bacterial cells expressing a heterologous lectin comprise at least one introduced cassette encoding the heterologous lectin, or at least two introduced cassettes encoding the same heterologous lectin or different heterologous lectins. For instance, in the case of one or more modified B. subtillis strains exemplified herein, the CZ438 strain comprises two introduced expression cassettes encoding mature Q-GRFT variant (SEQ ID NO: 2) for the secreted expression (Example 6). As further described in Example 15, cassettes encoding various other classes of lectin proteins were introduced into B. subtillis strain CBS12 and screened for lectin expression/production (TABLE 3).

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, heat treatment, ammonium sulphate precipitation or other protein salt precipitation, crystallization, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “protein preparation” is any material, typically a solution, generally aqueous, comprising one or more proteins.

As used herein, the terms “broth”, “cultivation broth”, “fermentation broth” and/or “whole fermentation broth” may be used interchangeably and refer to a preparation produced by cellular fermentation that undergoes no processing steps after the fermentation is complete. For example, whole fermentation broths are typically produced when microbial cultures are grown to saturation, incubated under carbon-limiting conditions to allow protein synthesis (e.g., expression of proteins by host cells; and optionally, secretion of the proteins into cell culture medium). Typically, the whole fermentation broth is unfractionated and comprises spent cell culture medium, metabolites, extracellular polypeptides, and microbial cells.

As used herein, the phrase “treated broth” refers to broth that has been conditioned by making changes to the chemical composition and/or physical properties of the broth. Broth “conditioning” may include one or more treatments such as cell lysis, pH modification, heating, cooling, addition of chemicals (e.g., calcium, salt(s), flocculant(s), reducing agent(s), enzyme activator(s), enzyme inhibitor(s), and/or surfactant(s)), mixing, and/or timed hold (e.g., 0.5 to 200 hours) of the broth without further treatment.

As used herein, a “cell lysis” process includes any cell lysis technique known in the art, including but not limited to, enzymatic treatments (e.g., lysozyme, proteinase K treatments), chemical means (e.g., ionic liquids), physical means (e.g., French pressing, ultrasonic), simply holding culture without feeds, and the like.

The terms “recovery”. “recovered” and “recovering” as used herein refer to at least partial separation of a protein from one or more components of a microbial broth and/or at least partial separation from one or more solvents in the broth (e.g., water or ethanol).

In certain aspects, broths in which host cells have been fermented for the production of lectin proteins, with or without broth treatment, are clarified. As used herein, a “clarified” broth means a broth which has been subjected to at least one clarification process to remove cell debris and/or other insoluble components. Clarification processes, as understood in the art include, but are not limited to, centrifugation techniques, cross-flow membrane filtration techniques, solid/liquid filtration techniques, and the like.

“Cell debris” refers to cell walls and other insoluble components that are released or formed after disruption of the cell membrane (e.g., after performing a cell lysis process).

In certain aspects, separation of solvents, as understood in the art include, but are not limited to ultrafiltration, evaporation, spray drying, freezer drying. The obtained solution is referred to as “clarified broth concentrate”, “UF concentrate”, or “ultrafiltrate concentrate”.

As used herein, the terms “lectin(s)” and “lectin proteins” are used interchangeably and refer to carbohydrate binding proteins (or glycoproteins) that can recognize and bind simple or complex carbohydrates in a reversible and highly specific manner, while displaying no catalytic activity. Thus, lectin proteins described herein have the same meaning as lectins described in art (e.g., see Lagarda-Diaz et al., 2017).

As used herein, the term “functional lectin variant(s)” and “when used in phrases such as a “native lectin and functional (lectin) variants thereof”, the “native griffithsin (GRFT) protein and functional GRFT variants thereof”, and the like, refers to variant (mutant) lectins derived from a native (parent) lectin protein, wherein the functional (lectin) variants comprise carbohydrate binding activity. In certain aspects, functional lectin variants may comprise reduced carbohydrate binding activity relative to the parent (native) lectin, the same carbohydrate binding activity relative to the parent (native) lectin, or increased carbohydrate binding activity relative to the parent (native) lectin.

### III. Lectin Proteins

As briefly set forth above, lectins are proteins (or glycoproteins) that possess non-catalytic carbohydrate-binding sites. As generally understood in the art, lectins differ from enzymes because their carbohydrate-binding properties never change, and they are unlike antibodies because they are not induced as an immune response. For example, some of the most well-known lectins are found in leguminous seeds, which are believed to be responsible for innate immunity and defense mechanisms in plants (Peumans and Van Damme, 1998). More recently, the use of lectins in mitigating viral infections (e.g., HIV, MERS-COV, SARS-COV-2, HCV, Ebola and the like) has received significant attention (PCT Publication No. WO2005/118627, WO2007/064844, WO2010/01424, WO2016/130628, WO2019/108656 and US Publication No. US20110263485). However, as described above in the Background, the economics of recombinant lectin production (e.g., using currently available host expression systems and downstream recovery process thereof) has significantly limited acceptance and/or use of lectins as anti-microbial compositions.

For example, PCT Publications WO2005/118627 and WO2007/064844 describe methods for isolating the native griffithsin (GRFT) lectin from the red algae (Griffithsia sp.), cloning the wild-type (grft) gene thereof, generating recombinant polynucleotides thereof, fermenting and producing the same in E. coli host cells, followed by isolating the recombinant His-tagged GRFT protein from the E. coli host, and characterizing its anti-viral activity. However, as described in WO2005/118627 and WO2007/064844, the recombinant GRFT protein (and a C-terminal His-tagged GRFT protein thereof) encoded by the nucleic acids of Example 2, did not efficiently translocate to the periplasmic fraction of E. coli following GRFT protein expression, wherein the majority of the produced GRFT proteins accumulated in the inclusion bodies of E. coli, without the cleavage of the pelB signal sequence located at the N-terminus of the griffithsin protein. Thus, steps were taken to express griffithsin in the cytosolic fraction of E. coli, using the N-terminal (His) tagged GRFT, or His-tagged variants of GRFT, as described above.

Likewise, PCT Publication No. WO2010/01424 generally describes methods of inhibiting a hepatitis C viral infection of a host comprising administering to the host an effective amount of a glycosylation resistant GRFT protein (or a polypeptide conjugate thereof) in combination with another anti-viral protein. For example, as described in this publication, the inventors noted that the anti-viral protein combination of scytovirin (SVN) and griffithsin (GRFT) have (nanomolar) activity against the Hepatitis C virus (HCV). US Patent Publication No. US20110263485 further describes methods of inhibiting a human immunodeficiency virus (HIV) viral infection of a host comprising administering to the host an effective amount of a gp120 Griffithsin and a peptide selected from a gp41-binding protein, a CCR5-binding protein, a gp120-binding protein, or another griffithsin, which combinations are potent inhibitors to HIV infection.

PCT Publication No. WO2016/130628 discloses variant griffithsin proteins having mutations that change the isoelectric point of the GRFT protein, which are reported to alter its solubility in various pH ranges allowing for improved product release.

PCT Publication No. WO2019/108656 generally describes microbicidal compositions comprising an endosperm extract and an anti-HIV lectin, an anti-HIV antibody, or antigen binding antibody fragment thereof. More particularly, the inventors utilized transgenic plants expressing two or more cyanovirin-N (CVN) proteins, griffithsin (GRFT) proteins, scytovirin (SVN) proteins, other anti-HIV lectin proteins. However, as stated in WO2019/108656, the production of such microbicidal components is expensive because fermenter based expression platforms are required, and the downstream processing facilities must be compliant with good manufacturing practice (GMP) to ensure the removal of viruses or endotoxins, wherein the capacity, scalability and cost issues affecting fermenters are exacerbated when two or three separate products with individual manufacturing processes are required for each microbicide.

The recombinant production of GRFT in tobacco plants (Nicotiana benthamiana) has been described by O'Keefe et al. (2009), wherein the GRFT accumulates to a level of about 1 gram of recombinant GRFT per kilogram of Nicotiana benthamiana leaf material, when expressed via an infectious tobacco mosaic virus (TMV) based vector.

Hirayama et al. (2016) have described the elucidated primary structure of KAA-2 lectin using peptide mapping and complementary DNA (cDNA) cloning and prepared its active recombinants using an E. coli expression system.

Gengenbach et al. (2019) have described the transient expression of the mistletoe lectin named “viscumin” (Viscum album) in intact Nicotiana benthamiana plants and purification of the recombinant viscumin from crude plant extracts by affinity chromatography, wherein the performance and economics of tobacco plant-based process was compared to the corresponding process based on E. coli expression. For example, as described in Gengenbach et al., the full-length viscumin was produce in N. benthamiana leaves at levels of up to 7 mg/kg of purified product, wherein the yield of full-length viscumin was comparable with that of plant lectins expressed in yeast, but approximately 6-fold lower than refolded viscumin A and B chains expressed in E. coli. As summarized by Gengenbach et al., the E. coli process has a low recovery, requires extensive dilution and is complex, whereas the plant-based process included only half the number of steps. According to a direct cost comparison between the two processes performed in the Gengenbach et al. study, the plant expression system was 50% less expensive, in comparison with the native host V. album or the heterologous expression in E. coli host cells.

As described herein, the instant disclosure addresses numerous ongoing and unmet needs in the art, particularly as related to the industrial scale production, recovery and/or purification of lectin proteins. More particularly, as described hereinafter and set forth below in the Examples, Applicant discloses novel end-to-end processes for the large-scale production, recovery, and purification of any recombinant lectin protein (or multiple lectin proteins) in a Gram-positive bacterial (host) cell. As set forth in Example 1, exemplary Gram-positive bacterial cells were designed, constructed, and evaluated for their ability to express heterologous (foreign) lectins, wherein Applicant has demonstrated that Gram-positive bacterial cells can express and produce significant amounts of a heterologous (i.e., eukaryotic) lectin known as griffithsin (GRFT), which was surprising and unexpected based on the current state of the art.

In other aspects, it was surprising and unexpected that Gram-positive bacterial cells can express and produce significant amounts of such heterologous (i.e., eukaryotic plant) lectins, which are known to agglutinate various Gram-positive bacterial cells (e.g., Bacillus sp. cells; Cole et al., 1984). For example, the jack-bean (Canavalia ensiformis) lectin Concanavalin-A (ConA) agglutinates Bacillus sp. cells (e.g., B. subtilis 168, Bacillus sphaericus, Bacillus amyloliquefaciens) by binding cell wall sugars (e.g., α-D-mannopyranosyl, α-D-glucopyranosyl and sterically related residues).

A study of legume (Leguminosiae) lectins (Ayouba et al., 1991) characterized other monosaccharides widely distributed on the cell wall of bacteria (e.g., Glc/Man-specific lectins, Gal/GalNAc-specific lectins, MurAc-sepcific lectins and MurNAc-specific lectins). As described by Ayouba et al. (1991), the interaction of these sugar residues may explain previously reported agglutination of various bacteria by legume lectins. Gram-positive cells have generally not been considered for use in the large-scale production of recombinant (eukaryotic) lectins proteins.

Thus, as contemplated and described herein, certain embodiments of the disclosure are related to, inter alia, nucleic acids encoding lectin proteins, recombinant cells expressing/producing one or more lectin proteins, the recovery of lectin proteins, the purification of lectin proteins, lectin (protein) preparations and the like. More particularly, in certain embodiments, native and/or variant lectin proteins and/or DNA (nucleic acid) sequences encoding the same, may be derived/obtained from known lectin proteins. In certain aspects, lectin proteins are derived from a host organism which naturally produces the lectin protein. Thus, in certain embodiments, a lectin protein of the disclosure is derived from a eukaryotic cell, a bacterial cell, or a cyanobacterial cell. In certain other one or more embodiments, a eukaryotic cell is photosynthetic plant cell, a red algae cell, an animal cell, or an insect cell.

In certain embodiments, a lectin protein is derived from one or more of the antiviral lectins described in US Patent Publication Nos. US20040204365, US20020127675, US20110189105 and US20110263485, and/or PCT Publication Nos. WO2005/118627, WO2008/022303, WO2010/014248, WO2014/197650, WO2016/130628 and WO2019/108656 (each incorporated herein by reference in its entirety). Thus, in certain aspects, a lectin protein is a scytovirin (SVN), a griffithsin (GRFT), a cyanovirin-N (CVN), functional fragments thereof, and/or functional variants (mutants) thereof.

In other embodiments, a lectin protein is one or more of the antiviral lectins described in PCT Publication No. WO2019/108656, such as the Artocarpus heterophyllus (jacalin) lectin, the Musa acuminata (banana) lectin, the Boodlea coacta lectin, the Microcystis viridis lectin, etc.) and/or functional fragments thereof, and/or functional variants thereof that retain the ability to bind to carbohydrates on viral envelopes described therein.

In other embodiments, a lectin protein is derived from a eukaryotic lectin source described in Singh and Sarathi (2012), including but not limited to, the Aaptos papilleta (Sponge) lectin, the Abrus precatorius (Jequirty bean) lectin, the Aegapodium podagraria (Ground elder) lectin, the Agaricus bisporus (Common mushroom) lectin, the Albizzia julibrissin (Mimosa tree seed) lectin, the Allomyrina dichotoma (Japanese beetle) lectin, the Aloe arborescens (Aloe plant) lectin, the Amphicarpaea bracteata (Hog peanut) lectin, the Anguilla (Eel) lectin, the Aplysia depilans (Mollusca; Mediterranean sea) lectin, the Arachis hypogaea (Peanut) lectin, the Artocarpus heterophullus (Jacalin) lectin, the Bauhinia purpurea (Camel's foot tree) lectin, the Bryonia diocia (White bryony) lectin, the Caragana Arborescens (Siberian pea tree) lectin, the Carcinoscorpius rotundacauda (Horseshoe crab) lectin, and/or functional fragments thereof, and/or functional variants thereof that retain the ability to bind to a specified carbohydrate (moiety) described in the Singh and Sarathi (2012) reference.

In certain aspects, a lectin protein of the disclosure may be classified into groups, including, but not limited to, “galactose (Gal)” specific lectins, “glucose (Glu)” specific lectins, “fucose (Fuc)” specific lectins, “mannose (Man)” specific lectins, “N-acetylgalactosamine (GalNAc)” specific lectins, “N-acetylglucosamine (GluNAc)” specific lectins, “sialic acid” specific lectins, and the like.

In certain embodiments, a lectin protein of the disclosure may be classified into groups based on the taxonomy of their origin species, including, but not limited to lectins derived from “red algae”, lectins derived from “plants”, lectins derived from “cyanobacteria”, lectins derived from “mammals”, lectins derived from “bacteria” and lectins derived from “fish”.

In other embodiments, one or more lectin proteins of the disclosure may be grouped or characterized according to a protein fold, such as a β-barrel, β-prism, β-trefoil and the like (e.g., see TABLE 3).

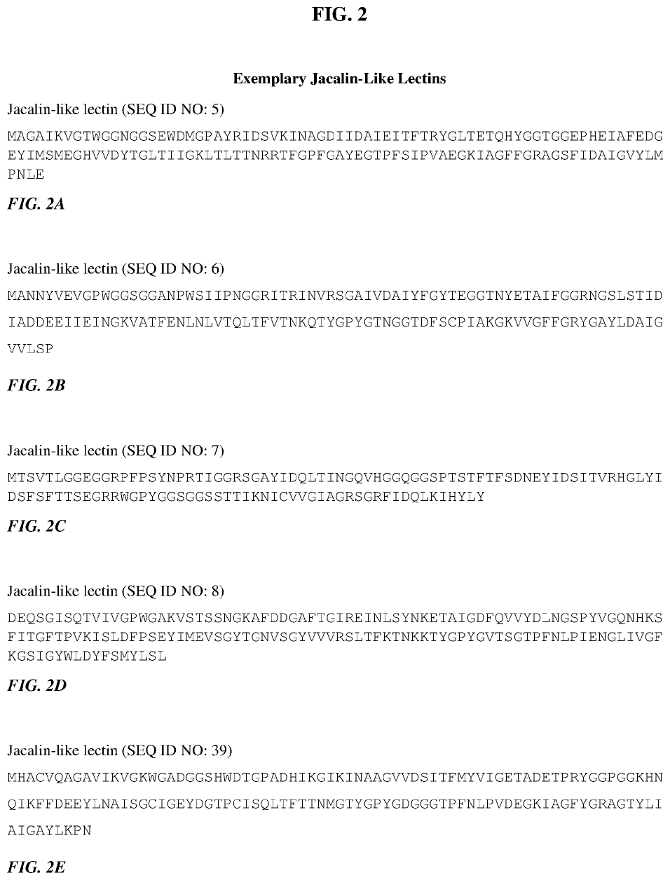

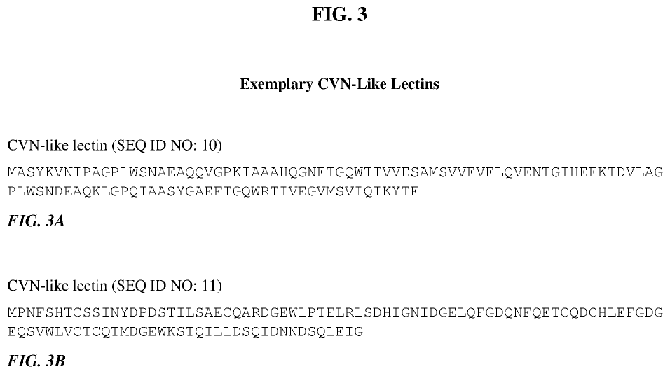

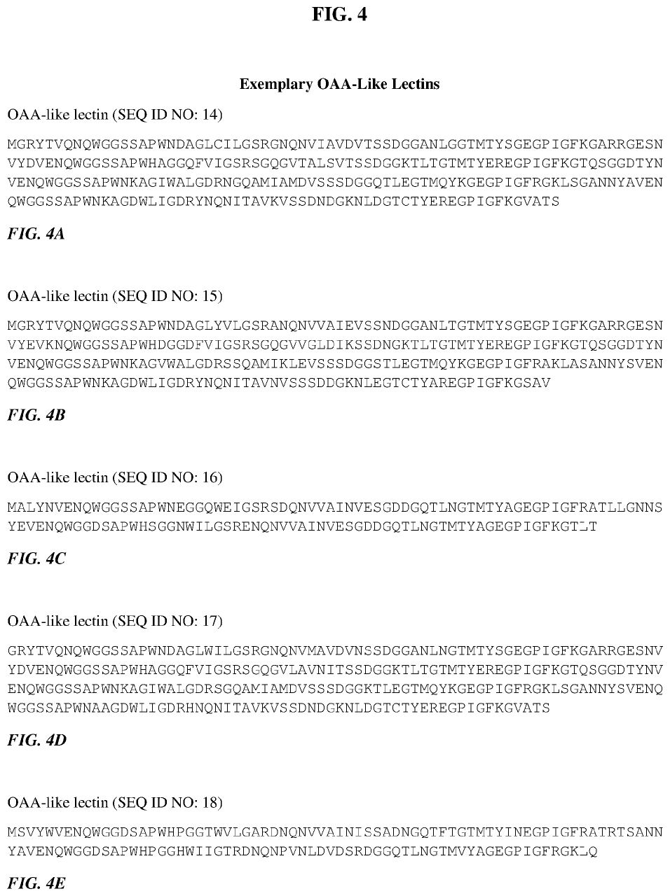

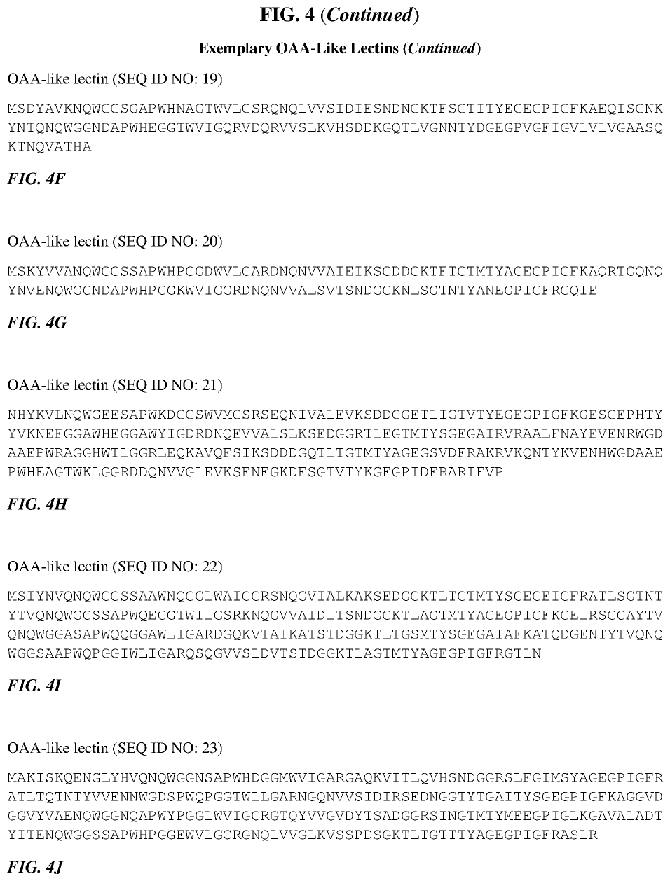

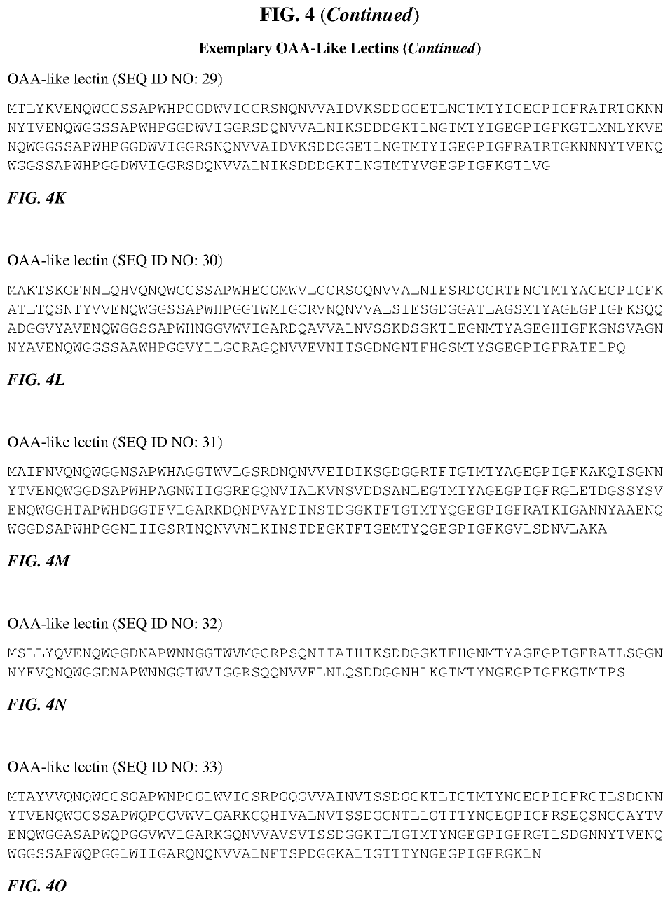

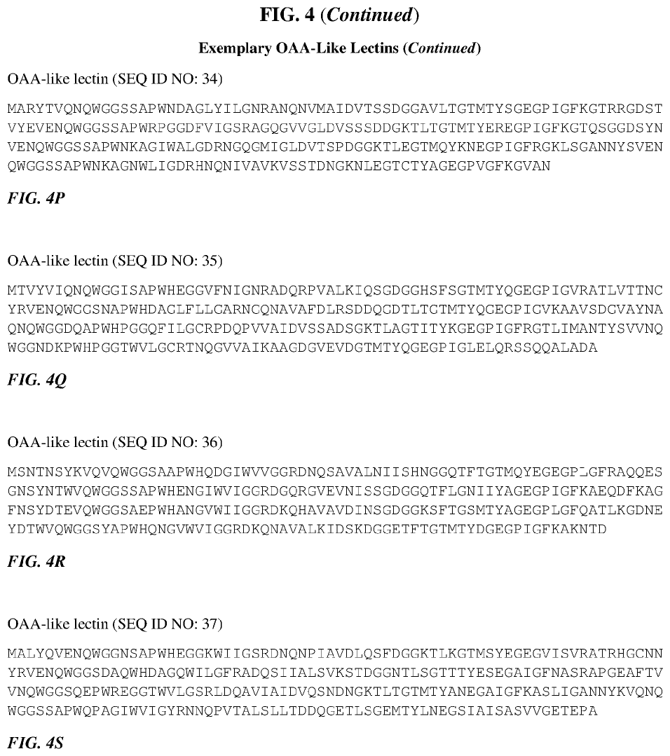

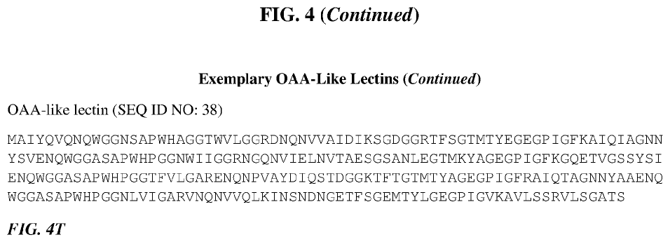

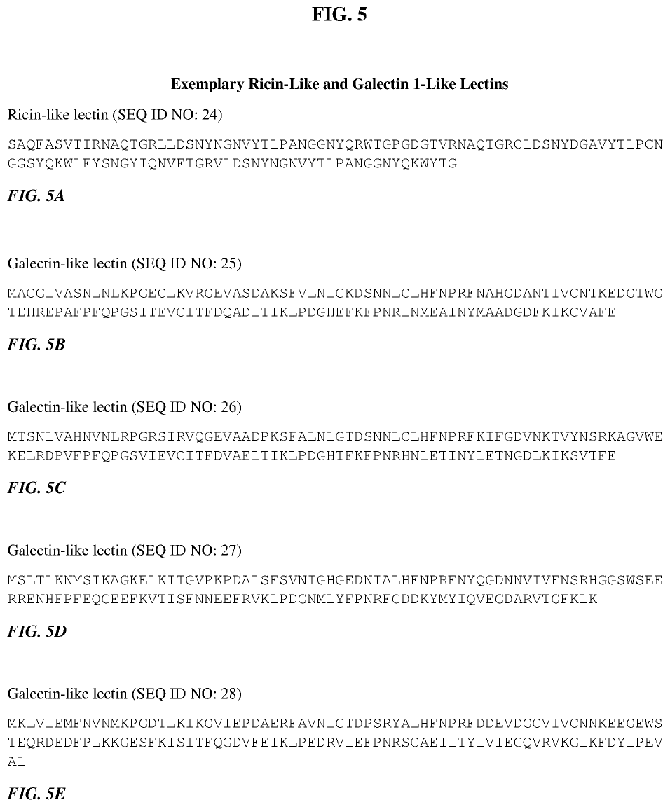

In certain other embodiments, one or more lectin proteins of the disclosure may be grouped or classified as a “jacalin-like” lectin (FIG. 2) a “CVN-like” lectin (FIG. 3), an “OAA-like” lectin (FIG. 4), a “ricin-like” lectin (FIG. 5) and a galactin-1-like lectin (FIG. 5).

Thus, in certain aspects, lectins suitable for use according of the instant disclosure may be derived/isolated from eukaryotic lectin source organisms. For example, in certain embodiments, a lectin (protein) can be isolated from the eukaryotic (source) organism using affinity chromatography processes known to one of skill in the art (i.e., one of the aforementioned carbohydrate moieties (Gal, Man, GalNAc, etc.) are attached the inert (chromatographic) matrix such that lectin proteins having binding specificity to the carbohydrate moiety will be retained.

In certain other embodiments, a lectin protein is a microvirin (MVN) lectin derived from the cyanobacterium Microcystis aeruginosa (PCC7806), which MVN lectin comprises mannose-specific affinity. For example, as described in Breitenbach Barroso Coelho et al. 2018 (incorporated herein by reference), the MVN lectin can inhibit HIV-1 infection, syncyntium formation between infected and uninfected CD4 T cells, and HIV-1 transmission. In other embodiments, a lectin protein is a scytovirin (SVN) derived from the cyanobacterium Scytonema varium, which binds with high affinity to mannose residues on the envelope glycoproteins of viruses and inhibits the virus replication, as observed with the Zaire Ebola virus (e.g., see Breitenbach Barroso Coelho et al., 2018). In yet other aspects, a lectin protein is a ESA-2 lectin derived from the red alga Eucheuma serra, which ESA-2 lectin exhibits anti-HIV activity and potent inhibition on influenza A virus (H1N1) infection (e.g., see Breitenbach Barroso Coelho et al., 2018). In another embodiment, a lectin protein is a BanLec (jacalin-related) lectin derived from the fruit of bananas (Musa acuminate), which recognizes high-mannose glycans found on viral envelopes such as HIV-1 (e.g., see Breitenbach Barroso Coelho et al., 2018).

In certain other embodiments, a lectin protein is a mannose-binding plant lectin derived from the rhizomes of Aspidistra elatior (AEL) which has been demonstrated to have significant in vitro inhibitory activity against the vesicular stomatitis virus, Coxsackie virus B4 and respiratory syncytial virus (e.g., see Breitenbach Barroso Coelho et al., 2018).

In certain other embodiments, a lectin protein is a CVL lectin (β-galactose-specific) derived from the marine worm Chaetopterus variopedatus having anti-HIV-1 activity (e.g., see Breitenbach Barroso Coelho et al., 2018).

In certain other embodiments, a lectin protein of the disclosure may be derived from the seeds of Vicia faba (fava bean), Lens culinaris (lentil), and Pisum sativum (pea), as generally described in El-Araby et al., 2020 (incorporated herein by reference). As generally set forth in the El-Araby et al. (2020) reference, crude extracts of the three leguminous were purified by affinity chromatography using mannose agarose, wherein the purified fava bean, lentil, and pea lectins comprised molecular weights of 18 kDa, 14 kDa, and 17 kDa, respectively, as determined by amino acid sequence analysis. For example, the minimum inhibitory concentration (MIC) values of these purified lectins when tested against bacteria (Pseudomonas aeruginosa, Staphylococcus aureus, Klebsiella pneumonia) and fungi (Candida albicans) ranged from 1.95 μg/ml to 250 μg/ml.

In another embodiment, a lectin protein of the disclosure is an antitumoral (anticancer) lectin derived from Viscum album (Mistletoe), such as the mistletoe lectin I (MLI) and mistletoe lectin III (MLIII) B-subunits described in Pevzner et al., 2004, and Gengenbach et al., 2019 (incorporated herein by reference).

As contemplated herein, native lectins and/or one or more variant lectins derived therefrom may be assessed for function or activity by means including, but not limited to, hemagglutination activity assays, carbohydrate/glycan binding affinity assays, antimicrobial inhibition assays, combinations thereof and the like, as set forth and described in El-Araby et al. (2020). Thus, in certain aspects, native lectins and/or variant lectins of the disclosure comprise antimicrobial activity (e.g., antiviral activity, antifungal activity, antibacterial activity).

In one or more other embodiments, a variant lectin comprises at least about 40% to about 99.9% sequence identity to a native (wild-type) lectin amino acid sequence. In certain other embodiments, a variant lectin comprises at least about 40% sequence identity to a native (wild-type) lectin amino acid sequence and comprises carbohydrate binding activity. In certain other embodiments, a variant lectin comprises at least about 40% sequence identity to a native (wild-type) lectin amino acid sequence and comprises an antimicrobial binding activity.

In other embodiments, a variant lectin is comprises at least about 40%, 41%, 42%, 43%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity to a native (wild-type) lectin protein of the disclosure.

In yet other embodiments, a variant lectin is comprises at least about 40%, 41%, 42%, 43%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity to a native or a variant lectin protein selected from the group consisting of SEQ ID NO: 1, SEQ ID NO: 2 and SEQ ID NO: 4-39.

In certain embodiments, a functional lectin protein comprises one or more non-catalytic carbohydrate-binding sites. Therefore, in certain one or more embodiments or aspects, a functional lectin protein may be characterized or assessed according to its carbohydrate-binding activity. In certain embodiments, native, variant and/or functional lectins may be described or defined by antimicrobial function/activity (e.g., antiviral activity, antifungal activity, antibacterial activity). In other one or more embodiments or aspects, native, variant and/or functional lectins may be screened or described by a hemagglutination activity assay and the like.

### IV. Gram-Positive Cells Producing Heterologous Lectins

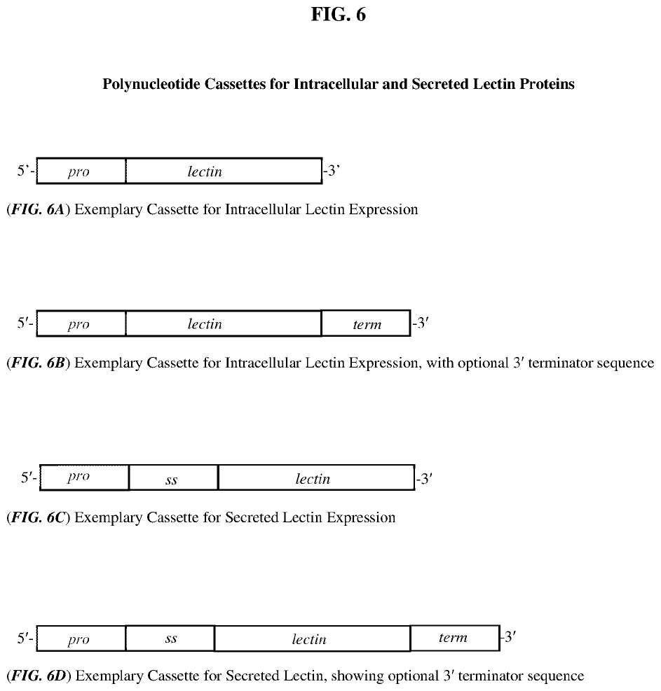

As briefly set forth above, and further described in Examples section below, Applicant has designed, constructed, and evaluated exemplary Gram-positive bacterial cells for their ability to express heterologous lectins. For example, in certain embodiments polynucleotides (expression cassettes; FIG. 6) encoding the variant Q-GRFT protein (Q-GRFT; SEQ ID NO: 2) were introduced into Bacillus cells and evaluated in large scale like bioreactors. More specifically, expression cassettes encoding the Q-GRFT (SEQ ID NO: 2) variant were constructed for intracellular expression and/or secreted expression of Q-GRFT in Bacillus cells and evaluated using large scale (˜10 L) bioreactors, as presented in Example 1. As described in this example, the exemplary Bacillus strains comprising either, the introduced cassette for secreted Q-GRFT expression, or the introduced cassette for intracellular Q-GRFT expression are shown in FIG. 6, which strains produced comparable amounts of Q-GRFT under the large-scale conditions tested, demonstrating that Gram-positive bacterial cells/strains are particularly useful host strains for large scale fermentation and production of lectin proteins.

In certain one or more embodiments, Applicant contemplates that the elimination or reduction of one or more (several) background enzyme (activities) will aid/facilitate lectin downstream recovery and purification processes described herein (e.g., by reducing undesired host cell background (native) protein contaminants). For instance, the use of Bacillus sp. strains deleted for endogenous enzymatic activities allows one to simply the fermentation media, by eliminating complex protein sources resulting in cleaner fermentation broth, faster and highly efficient recovery of the lectin protein. Thus, in certain embodiments, recombinant Gram-positive host cells are rendered deficient in the production of one or more endogenous genes encoding one or more highly expressed background proteins (e.g., an amylase, a xylanase, a pullulanase, a phytase, a pectate lyase, a beta-glucanase, a mannosidase, a lipase, an esterase and the like), which applicant contemplates will facilitate one or more lectin downstream recovery and purification processes described herein and/or reduce costs of one or more lectin downstream recovery and purification processes described herein (e.g., see Section VII).

For example, in the case of certain B. subtilis strains exemplified herein, the strains may be modified to be deficient in the production of one or more background enzymes (activities), such as an α-amylase (e.g., amyE), a protease (e.g., aprE), a β-glucanase (e.g., bglS), and the like. In particular, as set forth in the Examples below, certain B. subtilis strains of the disclosure have been modified to be deficient in the production of a native α-amylase (amyE). In other embodiments, B. subtilis strains of the disclosure are modified to be deficient in the production of a native protease.

In one or more particular embodiments, the B. subtilis strain CB462 comprises deletions of endogenous genes aprE, nprE (encoding background proteases aprE and nprE) and alrA (for use as selection marker), the B. subtilis strain CB460 comprises deletions of endogenous genes aprE, nprE, epr, isp, bpr, wprA (encoding background proteases aprE, nprE, epr, isp, bpr, wprA) and alrA (for use as selection marker), the B. subtilis strain CB447 comprises deletions of endogenous genes aprE, nprE, epr, isp, bpr, wprA, vpr, mpr, nprB (encoding background proteases aprE, nprE, epr, isp, bpr, wprA, vpr, mpr, nprB) and alrA (for use as selection marker), the B. subtilis strain CB476 comprises deletions of endogenous genes aprE, nprE, epr, isp, bpr, wprA, vpr, mpr, nprB (encoding background proteases aprE, nprE, epr, isp, bpr, wprA, vpr, mpr, nprB) and alrA (for use as selection marker), the B. subtilis strain CBS6 comprises deletions of endogenous genes aprE, nprE, epr, vpr, nprB (encoding background proteases aprE, nprE, epr, vpr, nprB), amyE (encoding background amylase AmyE) and scoC, the B. subtilis strain CZ438 comprises deletions of endogenous genes aprE, nprE, epr, isp, bpr, wprA, vpr, mpr, nprB (encoding background proteases aprE, nprE, epr, isp, bpr, wprA, vpr, mpr, nprB) and alrA (for use as selection marker), the B. subtilis strain CB488 comprises deletions of endogenous genes aprE, nprE, epr, isp, bpr, wprA, vpr, mpr, nprB (encoding background proteases aprE, nprE, epr, isp, bpr, wprA, vpr, mpr, nprB) and alrA (for use as selection marker), and the B. subtilis strain CBS12 comprises deletions of endogenous genes aprE, nprE, epr, isp, bpr, wprA, vpr, mpr, nprB (encoding background proteases aprE, nprE, epr, isp, bpr, wprA, vpr, mpr, nprB), amyE (encoding background amylasc AmyE), and scoC.

As further described in the Examples below (Example 15; TABLE 3), various classes of lectin proteins (e.g., jacalin-like lectins, CVN-like lectins, OAA-like lectins, galectin-1-like lectins, ricin-like lectins, and the like) representing different protein folds (e.g., β-barrel, β-sandwich, β-prism, etc.) were screened for expression/production in recombinant (modified) Gram-positive cells of the disclosure. More particularly, as demonstrated in the Examples, it was surprisingly overserved that Gram-positive bacterial cells/strains (e.g., Bacillus sp. cells) are particularly suitable host strains for the large-scale fermentation, production, and recovery of a diverse family of lectin proteins (TABLE 3).

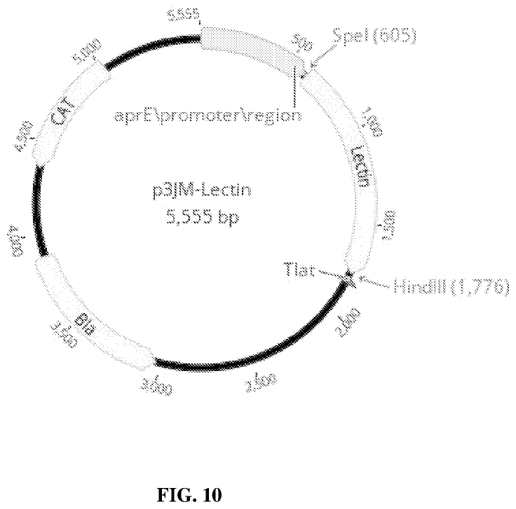

Thus, certain embodiments of the disclosure provide recombinant Gram-positive bacterial cells expressing one or more heterologous nucleic acids (polynucleotides) encoding lectin proteins. In certain embodiments, a recombinant Gram-positive bacterial cell expresses a heterologous polynucleotide encoding native a lectin protein, or a functional variant derived from the native lectin protein. In certain aspects, heterologous polynucleotides encoding lectin proteins are described as expression cassettes introduced into the recombinant cell. In certain embodiments, at least one expression cassette is introduced in the Gram-positive bacterial cell. In other embodiments, at least two expression cassettes are introduced in the Gram-positive bacterial cell. For example, FIG. 6 and FIG. 10 present schematic diagrams of exemplary lectin polynucleotide cassettes suitable for intracellular expression (e.g., FIG. 6A-6B) and/or extracellular expression/secretion (e.g., FIG. 6C-6D) of lectin proteins.

Thus, in certain one or more embodiments or aspects, Gram-positive host cells of the disclosure comprise one or more lectin expressions cassette introduced therein, wherein the host cells express the lectins when cultivated under suitable conditions. In certain other one or more embodiments, recombinant cells of the disclosure comprise at least two introduced expression cassettes encoding the same heterologous lectin, or at least two introduced expression cassettes encoding different (heterologous) lectins. In certain other one or more embodiments or aspects of the disclosure, recombinant cells comprise one or more introduced expression cassettes encoding a lectin for intracellular expression and/or one or more introduced expression cassettes encoding a lectin for secreted (extracellular) expression and combinations thereof. For example, recombinant cells of the disclosure may be constructed to express both intracellular and secreted lectins, which lectins may be recovered and purified according to one or more methods/processes described herein.

In certain related embodiments, Gram-positive bacterial cells include the classes Bacilli, Clostridia and Mollicutes (e.g., including Lactobacillies with the families Acrococcaccac, Carnobacteriaceae, Enterococcaceae, Lactobacillaceae, Leuconostocaceae, Oscillospiraceae, Streptococcaceae and the Bacillales with the families Alicyclobacellaceae, Bacillaceae, Caryophanaceae, Listeriaceae, Paenibacillaceae, Planococcaceae, Sporolactobacillus, Staphylococcaceae, Thermoactinomycetaceae, Turicibacteraceae).

In certain embodiments, species of the family Bacillaceae include Alkalibacillus, Amphibacillus, Anoxybacillus, Bacillus, Caldalkalibacillus, Cerasilbacillus, Exiguobacterium, Filobacillus, Geobacillus, Gracilibacillus, Halobacillus, Halolactibacillus, Jeotgalibacillus, Lentibacillus, Marinibacillus, Oceanobacillus, Ornithinibacillus, Paraliobacillus, Paucisalibacillus, Pontibacillus, Pontibacillus, Saccharococcus, Salibacillus, Salinibacillus, Tenuibacillus, Thalassobacillus, Ureibacillus, Virgibacillus.

In other embodiments, a Bacillus sp. cell includes, but is not limited to, B. acidiceler, B. acidicola, B. acidocaldarius, B. acidoterrestris, B. aeolius, B. aerius, B. aerophilus, B. agaradhaerens. B. agri, B. aidingensis, B. akibai, B. alcalophilus, B. algicola, B. alginolyticus, B. alkalidiazo-trophicus, B. alkalinitrilicus, B. alkalitelluris, B. altitudinis, B. alveayuensis, B. alvei, B. amylolyticus, B. aneurinilyticus, B. aneurinolyticus, B. anthracia, B. aquimaris, B. arenosi, B. arseniciselenatis, B. arsenicoselenatis, B. arsenicus, B. arvi, B. asahii, B. atrophaeus, B. aurantiacus, B. axarquiensis, B. azotofixans, B. azotoformans, B. badius, B. barbaricus, B. bataviensis, B. beijingensis, B. benzoevorans, B. bogoriensis, B. boroniphilus, B. borstelenis, B. butanolivorans, B. carboniphilus, B. cecembensis, B. cellulosilyticus, B. centrosporus, B. chagannorensis, B. chitinolyticus, B. chondroitinus, B. choshinensis, B. cibi, B. circulans, B. clarkii, B. clausii, B. coagulans, B. coahuilensis, B. cohnii, B. curdianolyticus, B. cycloheptanicus, B. decisifrondis, B. decolorationis, B. dipsosauri, B. drentensis, B. edaphicus, B. ehimensis, B. endophyticus, B. farraginis, B. fastidiosus, B. firmus, B. plexus. B. foraminis, B. fordii, B. formosus, B. fortis, B. fumarioli, B. funiculus, B. fusiformis, B. galactophilus, B. galactosidilyticus, B. gelatini, B. gibsonii, B. ginsengi. B. ginsengihumi, B. globisporus, B. globisporus subsp. globisporus, B. globisporus subsp. marinus, B. glucanolyticus, B. gordonae, B. halmapalus, B. haloalkaliphilus, B. halodenitrificans, B. halodurans, B. halophilus, B. hemicellulosilyticus, B. herbersteinensis, B. horikoshii. B. horti, B. hemi, B. hwajinpoensis, B. idriensis, B. indicus, B. infantis, B. infernus, B. insolitus, B. isabeliae, B. jeotgali, B. kaustophilus, B. kobensis, B. koreensis, B. kribbensis, B krulwichiae, B. laevolacticus, B. larvae, B. laterosporus, B. lautus, B. lehensis, B. lentimorbus, B. lentus, B. litoralis, B. luciferensis, B. macauensis. B. macerans, B. macquariensis, B. macyae, B. malacitensis, B. mannanilyticus, B. marinus, B. marisflavi, B. marismortui, B. massiliensis, B. methanolicus, B. migulanus, B. mojavensis, B. mucilaginosus, B. muralis, B. murimartini, B. mycoides, B. naganoensis, B. nealsonii, B. neidei. B, niabensis, B. niacini. B. novalis, B. odysseyi, B. okhensis, B. okuhidensis, B. oleronius, B. oshimensis, B. pabuli, B. pallidus, B. pallidus (illeg.), B. panaciterrae. B. pantothenticus, B. parabrevis, B. pasteurii, B. patagoniensis, B. peoriae, B. plakortidis, B. pocheonensis, B. polygoni, B. polymyxa, B. popilliae, B. pseudalcaliphilus, B. pseudofirmus, B. pseudomycoides, B. psychrodurans, B. psychrophilus, B. psychrosaccarolyticus, B. psychrotolerans, B. pulvifaciens, B. pycnus, B. qingdaonensis, B. reuszeri, B. runs, B. safensis, B. salarius, B. salexigens, B. saliphilus, B. schlegelii, B. selenatarsenatis, B. selenitrireducens, B. seohaeanensis, B. shackletonii, B. silvestris, B. simplex, B. siralis, B. smithii, B. soli, B. sonorensis, B. sphaericus, B. sporothermodurans, B. stearothermophilus, B. stratosphericus, B. subterraneus, B. subtilis subsp. spizizenii. B. subtilis subsp. subtilis, B. taeanensis, B. tequilensis, B. thermantarcticus, B. thermoaerophilus, B. thermoamylovorans, B. thermoantarcticus, B. thermocatenulatus, B. thermocloacae, B. thermodenitrificans, B. thermoglucosidasius, B. thermoleovorans, B. thermoruber, B. thermosphaericus, B. thiaminolyticus, B. thioparans, B. thuringiensis, B. tusciae, B. validus, B. vallismortis, B. vedderi, B. velezensis, B. vietnamensis, B. vireti, B. vulcani, B. wakoensis and B. weihenstephanensis.

In a particular embodiment, the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. As used herein, the “Bacillus genus” include Bacillus sp. that have been reclassified, including, but not limited to B. stearothermophilus, which is now named “Geobacillus stearothermophilus”.

### V. Molecular Biology

As generally set forth above and further described below in the Examples, certain embodiments of the disclosure are related to recombinant Gram-positive bacterial cells expressing heterologous lectin proteins, recombinant polynucleotides (e.g., vectors, expression cassettes) encoding heterologous lectin proteins particularly suitable for introducing (e.g., transforming) into Gram-positive host cells (i.e., for the expression of heterologous lectins) and the like. In other embodiments, Gram-positive host cells of the disclosure are constructed (rendered) to be deficient in the production of one or more endogenous proteins (e.g., enzymes). For example, in one or more embodiments or aspects of the disclosure, Applicant contemplates that the elimination or reduction of one or more (several) background enzyme (activities) will aid/facilitate lectin downstream recovery and purification processes described herein (e.g., by reducing undesired host cell background (native) protein contaminants).

Thus, in one or more embodiments or aspects, recombinant host cells may comprise genetic modifications (e.g., deletions, disruptions, interfering RNA, etc.) of one or more endogenous genes encoding one or more native background proteins (e.g., glycoside hydrolases native to the recombinant cell, proteases native to the recombinant cell, and the like). In certain one or more embodiments, recombinant cells of the disclosure are rendered deficient in the production of one or more endogenous proteins including, but not limited to, amylases, pullulanases, xylanases, proteases, and the like. For example, in the case of recombinant B. subtilis cells expressing one or more lectin proteins, the cells may be rendered deficient in the production of one or more endogenous proteins, including but not limited to, α-amylases (e.g., amyE), β-glucanases (e.g., bglS), esterases (e.g., pnbA), lipases (e.g., lipA), mannosidases (e.g., gmuG), pectate lyases (e.g., pel), phytases (e.g., phy), proteases (e.g., aprE, nprE, etc.), pullulanases (e.g., amyX) and the like.

Thus, certain embodiments of the disclosure are related to, inter alia, nucleic acids, polynucleotides (e.g., plasmids, vectors, expression cassettes), regulatory elements, and the like, suitable for use in constructing recombinant Gram-positive host cells. Accordingly, as presented in the Examples and generally described herein, recombinant cells of the disclosure may be constructed by one of skill using standard and routine recombinant DNA and molecular cloning techniques well known in the art. Methods for genetically modifying cells include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) a gene down-regulation, (f) site specific mutagenesis and/or (g) random mutagenesis.

In certain embodiments, recombinant (modified) cells of the disclosure may be constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified cell is constructed by gene deletion to eliminate or reduce the expression of the gene. Gene deletion techniques enable the partial or complete removal of the gene(s), thereby climinating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. By way of example, the contiguous 5′ and 3′ regions may be introduced into a Bacillus cell (e.g., on a temperature-sensitive plasmid such as pE194) in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is affected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers. Thus, a person of skill in the art may readily identify nucleotide regions in the gene's coding sequence and/or the gene's non-coding sequence suitable for complete or partial deletion.

In other embodiments, a modified cell is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is affected by selection for the marker under conditions not permitting plasmid replication. Selection for a second recombination event leading to gene replacement is affected by examination of colonies for loss of the selectable marker and acquisition of the mutated gene. Alternatively, the defective nucleic acid sequence may contain an insertion, substitution, or delction of one or more nucleotides of the gene, as described below.

In other embodiments, a modified cell is constructed by established anti-sense techniques using a nucleotide sequence complementary to the nucleic acid sequence of the gene. More specifically, expression of the gene by a host cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to, RNA interference (RNAi), small interfering RNA (siRNA), microRNA (miRNA), antisense oligonucleotides, and the like, all of which are well known to the skilled artisan.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions and selecting for mutant cells exhibiting reduced or no expression of the gene.

PCT Publication No. WO2003/083125 discloses methods for modifying Bacillus cells. such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) markcr-less delction.

Those of skill in the art are well aware of suitable methods for introducing polynucleotide sequences into bacterial cells (e.g., E. coli, Bacillus sp.). Indeed, such methods as transformation including protoplast transformation and congression, transduction, and protoplast fusion are known and suited for use in the present disclosure. Methods of transformation are particularly suitable to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Gram-positive bacterial strain by methods known in the art. In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Gram-positive cells are generally known on one of skill in the art. Promoter sequences of the disclosure are generally chosen so that they are functional in the Gram-positive host cells (e.g., Bacillus cells such as B. licheniformis cells, B. subtilis cells, B. amyloliquefaciens and the like). For example, promoters useful for driving gene expression in Bacillus cells include, but are not limited to, the B. subtilis alkaline protease (aprE) promoter, the α-amylase promoter (amyE) of B. subtilis, the α-amylase promoter (amyL) of B. licheniformis, the α-amylase promoter of B. amyloliquefaciens, the neutral protease (nprE) promoter from B. subtilis, a mutant aprE promoter, or any other promoter from B. licheniformis or other related Bacilli. Methods for screening and creating promoter libraries with a range of activities (promoter strength) in Bacillus cells is described in PCT Publication No. WO2002/14490.

### VI. Cultivating Gram-Positive Cells for the Production of Lectins

In certain embodiments, the present disclosure provides recombinant cells capable of producing lectin proteins of interest. More particularly, certain embodiments are related genetically modified (recombinant) Gram-positive bacterial cells expressing heterologous lectins. Thus, particular embodiments are related to cultivating (fermenting) Gram-positive cells for the production of lectin proteins. In general, fermentation methods well known in the art are used to ferment the Gram-positive cells.

In some embodiments, the cells are grown under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as temperature. pH, and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in post log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen, and the partial pressure of waste gases, such as CO₂, O₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Culturing/fermenting is generally accomplished in a growth medium comprising an aqueous mineral salts medium, organic growth factors, a carbon and energy source material, molecular oxygen, and, of course, a starting inoculum of the microbial host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for most Gram-positive cells the temperature generally will be within the range of about 20° C. to 40° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. Preferences for pH range of microorganisms are dependent on the media employed to some extent, as well as the particular microorganism, and thus change somewhat with change in media as can be readily determined by those skilled in the art.

In certain aspects, the fermentation is conducted in such a manner that the carbon-containing substrate can be controlled as a limiting factor, thereby providing good conversion of the carbon-containing substrate to cells, and avoiding contamination and inhibition of the cells with a substantial amount of unconverted substrate. The latter is not a problem with water-soluble substrates since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen, or carbon dioxide in the off-gases from the fermenter, cell density measurable by dry cell weights, light transmittance, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating, or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

### VII. Lectin Recovery and Purification Processes

As further detailed hereinafter and presented in the Examples, the instant disclosure further describes and exemplifies particularly suitable processes (methods) for harvesting, clarifying, recovering, purifying and the like fermentation broths in which one or more lectin proteins have been produced. Thus, certain embodiments are related to, inter alia, collecting broths at the end of fermentation, harvesting collected broths, recovering one or more lectins from a harvested broth (e.g., such as clarifying harvested broths, concentrating clarified broths, purifying clarified broth concentrates, and the like). In certain aspects, purified lectin (protein) preparations are derived from fermentation broths collected and harvested as described herein.

Certain other aspects of the disclosure provide, inter alia, novel methods for the recovering and optionally purifying lectins obtained from a recombinant cell expressing a lectin (e.g., a recombinant Gram-negative cell, a recombinant Gram-positive cell, a recombinant a plant (e.g., tobacco) cell, and the like). Certain other aspects of the disclosure provide, inter alia, novel methods for the recovery and optional purification of lectins obtained from naturally occurring sources (e.g., see Section III, Lectin Proteins).

Thus, in certain aspects, a lectin protein preparation is recovered according to the compositions and methods of the disclosure. In other aspects, a lectin preparation is recovered and purified according to the methods of the disclosure. As used herein, the terms “purified”, “isolated” or “enriched” with regard to a “lectin” (protein) means that the lectin is transformed from a less pure state by virtue of separating it from some, or all of, the contaminants with which it is associated. Contaminants include, but are not limited to, microbial cells, metabolites, solvents, chemicals, color, inactive forms of the target lectin, aggregates, process aids, inhibitors, fermentation media, cell debris, nucleic acids, proteins other than the target lectin. host cell proteins, cross-contaminants from the production equipment and the like.

Thus, in the context of a “purified lectin” as used herein, purification may be accomplished by any art-recognized separation techniques, including, but not limited to, ion exchange chromatography, affinity chromatography, hydrophobic separation, dialysis, protease treatment, heat treatment, ammonium sulphate precipitation or other protein salt precipitation, crystallization, centrifugation, size exclusion chromatography, filtration, microfiltration, gel electrophoresis, or separation on a gradient to remove whole cells, cell debris, impurities, extraneous proteins, or enzymes undesired in the final composition.

It is further possible to then add constituents to a purified or isolated lectin composition which provide additional benefits, for example, activating agents, anti-inhibition agents, desirable ions, compounds to control pH or other enzymes or chemicals.

As used herein, lectin “purity” is a relative term, and is not meant to be limiting, when used in phrases such as a “recovered lectin is of higher purity, the same purity, or lower purity than prior to the recovery process”. For example, the relative “purity” of a lectin (protein), before and after a recovery process, may be determined using methods known in the art, including but not limited to, general quantification methods (e.g., Bradford, UV-Vis, activity assays), electrophoretic analysis (SDS-PAGE), analytical HPLC, mass spectrometry, hydrophobic interaction chromatography and the like.

Non-limiting examples for accessing the relative purity of a lectin, include, but are not limited to, SDS-PAGE analysis and/or the A₂₈₀ ratio of non-lectin (impurities) relative to lectin.

For example, the relative lectin purity via SDS-PAGE can be determined by visual abundance of lectin (protein) band compared to non-lectin protein (unwanted contaminants; impurities) bands present in the preparation. Alternatively, the relative purity of a lectin can be determined by the non-lectin to lectin (A₂₈₀) ratio. For example, the A₂₈₀ ratio is a measure of amount of 280 nm absorbance contributed by non-lectin impurities for 1 unit 280 nm absorbance contributed by lectin in a protein preparation (e.g., non-lectin A₂₈₀/lectin A₂₈₀), wherein a smaller number means higher purity. More particularly, the method for determining the lectin (A₂₈₀) concentration can be measured by HPLC using a purified lectin as the standard, wherein the concentration by HPLC is converted to lectin A₂₈₀ using 1 mg/ml lectin=0.936 at 280 nm absorbance. The method for determining the non-lectin (A₂₈₀) concentration in the preparation can be measured using a 1 cm path glass cuvette zeroed with MilliQ water, diluted to A₂₈₀<1 with MilliQ water as needed, wherein non-lectin A₂₈₀ concentration is calculated by subtracting the lectin A₂₈₀ from the preparation measurement.

Thus, according to certain aspects, lectin (protein) preparations are recovered from fermentation broths, wherein the recovered lectin preparations are of higher purity after performing one or more recovery processes described herein. For example, a fermentation broth (e.g., a whole broth at the end of fermentation) may be subjected to one or more protein recovery processes including, but not limited to, broth conditioning processes, broth clarification processes, protein enrichment and/or protein purification processes (e.g., protein concentration, filtration, precipitation, crystallization, crystal separation, crystal sludge dissolution processes and the like), buffer exchange processes, sterile filtration processes and the like. In certain aspects, the fermentation broth is subjected to a broth treatment (broth conditioning) process to improve subsequent broth handling properties.

In certain embodiments, such as when a Gram-positive host cell has been constructed for intracellular lectin expression, a fermentation broth is subjected to a cell lysis process. For example, cell lysis processes include without limitation, enzymatic treatments (e.g., lysozyme, proteinase K treatments), chemical means (e.g., ionic liquids), physical means (e.g., French pressing, ultrasonic), simply holding culture without feeds, and the like. Thus, in certain preferred embodiments, a broth lysis process releases intracellular lectins into the (lysed) cell broth.

Thus, as described herein, the methods/processes of the disclosure are not meant to be limiting, as one of skill may readily adapt or modify one or more of the compositions and/or methods disclosed herein for the recovery of specific lectin proteins, and/or combinations thereof. In certain aspects, a fermentation broth obtained by fermenting Gram-positive cells expressing secreted lectins can be processed by harvesting, clarifying, and concentrating the broth, as generally described in Example 2. More particularly, as set forth in Example 2, the lectin (Q-GRFT) recovery yield of the concentrate was 65%, with substantial purity vis-à-vis the broth supernatant.

Likewise, as demonstrated in Examples 3 and 4, fermentation broths harvested, clarified and/or concentrated as above, may be further enriched, or purified by means of pH treatments and/or heat treatments. More specifically, as described in Example 3, ultrafiltration (UF) concentrates prepared in Example 2, were treated at various pH and temperature ranges, which UF concentrate samples were subsequently centrifuged, and the supernatants collected for analysis by SDS-PAGE. As presented in this example, all pH treated supernatants (i.e., at 5° C., 22° C. and 55° C.) have higher purity than initial whole broth sample.

Additionally, as described in Example 4, ultrafiltration (UF) concentrates from Example 2, were subjected to a low pH and heat treatments, further comprising the addition of activate carbon, wherein the low pH/heat treated samples were subsequently filtered and the collected filtrate analyzed for purity by SDS-PAGE. Significant purification of Q-GRFT was achieved with low pH treatment (adjustment) followed by filtration, wherein sample incubation at 60° C. further improved protein purity, and the addition of activated carbon further reduced color of the collected filtrate (data not shown).

In certain other embodiments, a fermentation broth obtained by fermenting a Gram-positive cell expressing an intracellular lectin protein is processed by harvesting, lysing, clarifying, and concentrating the broth, as generally described in Example 5. As described in this example, fermentation broths were harvested at the end of fermentation and treated with lysozyme, pH/heat treatments performed and the cooled broth was collected, wherein the collected broth was recovered using the same microfiltration and ultrafiltration procedures outlined and described in Example 2. More particularly, as described in Example 5, a visually clear concentrate was produced, wherein the heat-treated broth supernatant provided a significant purity improvement over the untreated broth supernatant, wherein the Q-GRFT purity remained through the processing steps, e.g., microfiltration for cell separation and ultrafiltration (UF) for dewatering (data not shown).

In certain other embodiments, fermentation broths of the disclosure are processed as described in Example 6. More particularly, fermentation broths of Gram-positive bacterial cells comprising two (2) introduced copies of the Q-grft expression cassette were processed using the same lysozyme treatment, pH adjustment and heat treatment processes described above (see, Example 5), wherein the broth was recovered as described in Example 6. More particularly, as described in Example 6, a visually clear concentrate was produced, wherein the pH adjusted and heat-treated broth supernatants demonstrated significant purity improvements over untreated broth supernatants (data not shown). Likewise, the Q-GRFT purity remained through the processing steps, flocculation and Buchner filtration for cell separation and ultrafiltration (UF) for dewatering. The obtained UF concentrate has purity similar to that of Example 5, derived from similarly treated broth, but recovered using microfiltration for cell separation and higher purity than untreated broth derived UF concentrate (Example 2).

In yet other embodiments, a fermentation broth obtained by fermenting Gram-positive cells expressing secreted lectins is processed by harvesting, clarifying, and concentrating the broth, as generally described in Example 7. As described in this example, fermentation broth was held at the end of fermentation for a sufficient amount of time (under conditions specified) with gentle mixing, followed by a heat treatment for a sufficient amount of time (with pH adjustment as needed), the broth was then cooled and collected, and recovered as described in Example 6. In particular, the pH adjusted and heat-treated broth supernatants provided significant purity improvements over untreated broth supernatants, wherein the Q-GRFT purity remained through the processing steps (e.g., flocculation and Buchner filtration for cell separation and ultrafiltration (UF) for dewatering (data not shown).

In other embodiments, one or more broth concentrates of the disclosure may be subjected to one or more protein purification processes. More specifically, as described above and set forth in Examples 8-10, one or more protein purification processes include protein crystallization processes, wherein all of the concentrates processed according to Example 8, gave square to rectangular plate shaped crystals (data not shown), all of the concentrates processed according to Example 9, formed crystals in the filtrates (see, TABLE 1). and all of the concentrates processed according to Example 10, formed square to rectangular plate shaped crystals in the filtrates (data not shown).

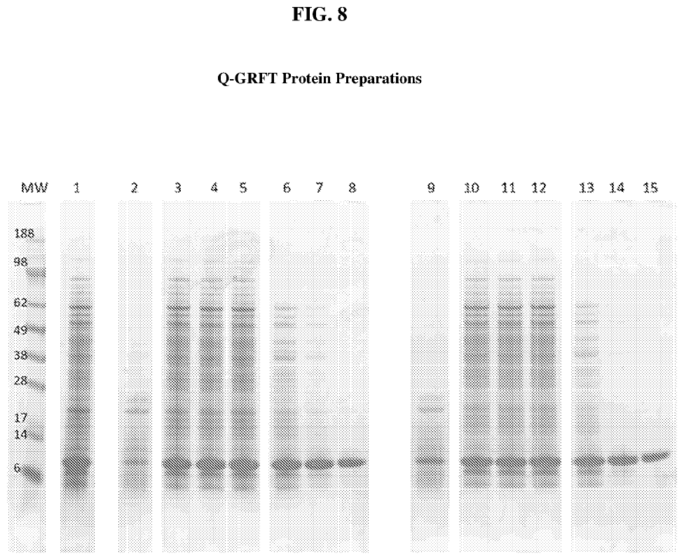

In other aspects, the disclosure provides methods and compositions for producing or obtaining high purity lectin (protein) preparations. More particularly, as set forth in Example 11, crystal slurries were readily derived from broth concentrates, which may include one or more crystal washing steps. As described in this example, after about 48 hours of crystallization, the crystal slurries were centrifuged, and the supernatant decanted, demonstrating a substantial crystallization yield of about 92%. More particularly, the purification factors were about 2.1 to 3.3 times improved over initial UF concentrates (see, TABLE 2). As presented in FIG. 8, high purity protein preparations were obtained by dissolving crystal pellets in sodium acetate, followed by filtration, wherein Q-GRFT was the only visible band by SDS-PAGE, in both the no wash and 1× wash dissolved filtrates (see, FIG. 8). Similarly, Example 12 describes exemplary recovery of crystals and crystal pellet dissolution of various clarified broth concentrates of the disclosure, wherein single band or near single band purity filtrates were obtained.

In other embodiments, broth concentrates of the disclosure are crystallized as generally described in Example 13, wherein suitable crystals formed in the solutions with and without salt addition at under conditions specified.

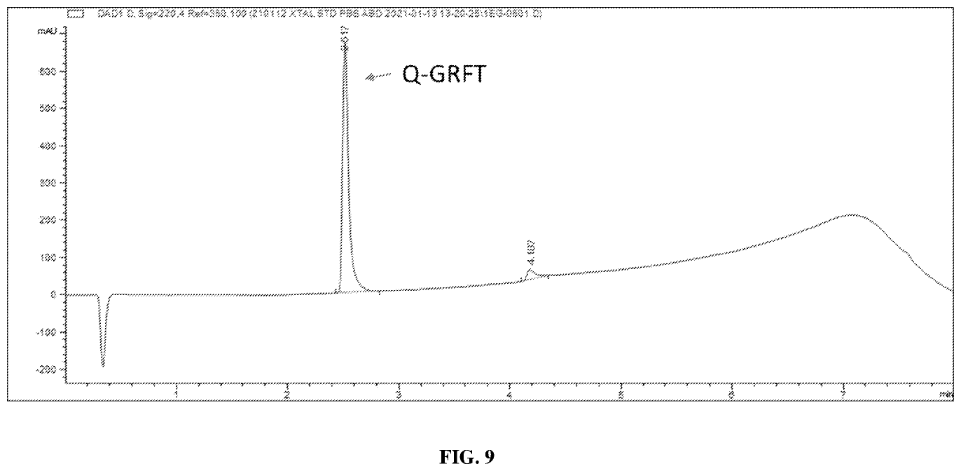

In certain other embodiments, the disclosure provides methods for obtaining/recovering high purity lectin protein preparations, as generally described in Example 14. More particularly, as described in this example, high purity lectin preparations were obtained with liquid-liquid (two-phase) extractions, wherein the lectin protein preferentially partitions into one of the fractions. For example, lectin (Q-GRFT) preparations obtained/recovered by such two-phase extraction methods (Example 14; see FIG. 9), were greater than 90% pure.

As further presented and described below in Examples 15-17, recombinant Gram-positive cells of the disclosure can express/produce various classes of lectins, including, but not limited to, jacalin-like lectins, CVN-like lectins, OAA-like lectin, ricin-like lectins, galactin-1-like lectins and the like. More particularly, as set forth in Examples 15 and 16, one or more lectins described herein may be produced in recombinant (modified) Gram-positive cells of the disclosure for large-scale fermentation, production, recovery, and optional purification of the one or more lectins expressed/produced.

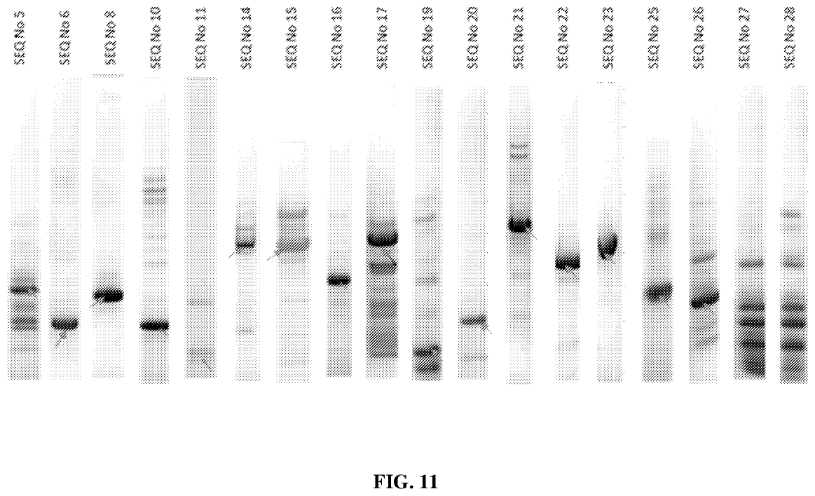

For instance, as described in Example 15, expression plasmids for thirty-five (35) diverse lectins were generated based on the B. subtilis strain CBS12 and their expression was evaluated in 2.5 L ultra-yield flasks. In particular, SDS-Page analysis of the supernatants from these strains revealed significant expression of many diverse lectins that embody various protein folds, molecular weights, sugar-binding specificities, and origin species (FIG. 11).

As described in Example 16, recombinant lectins produced as described above can be fermented, recovered, and purified using exemplary purification methods such as acid treatment, isoelectric precipitation and ion affinity separation via a chromatography column (Example 16, TABLE 4).

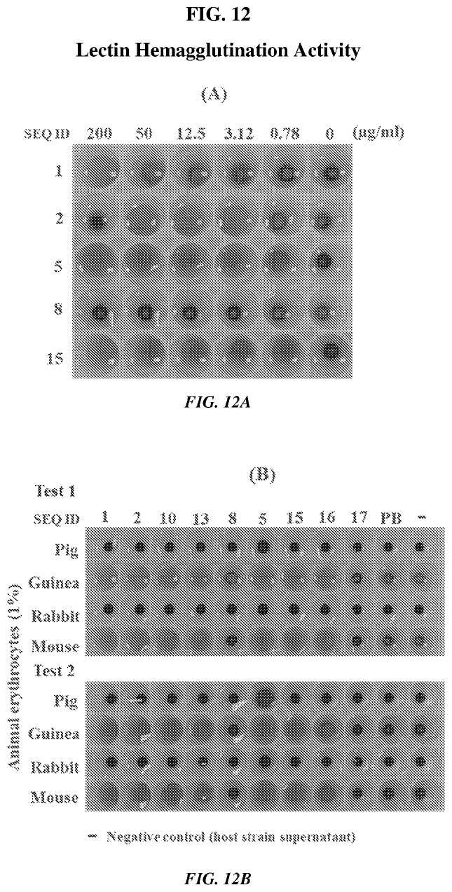

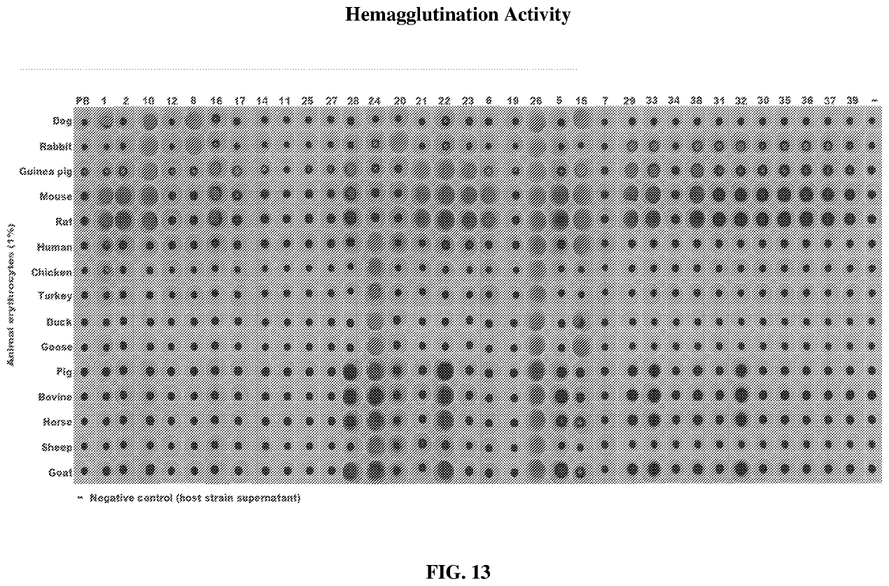

The produced (purified or crude) lectins were tested for functional activity using a hemagglutination assay as described in Example 17. For example, active lectins react with specific carbohydrate moieties on red blood cell surfaces (resulting in the formation of a diffuse matrix), while non-active lectins cannot bind red blood cells (resulting in the formation of noticeable clumps), which allows for a clear distinction between active and non-active samples (e.g., FIG. 12). As described in this example, lectin hemagglutination capability was evaluated using erythrocytes from 15 sources (FIG. 13). It is expected that various lectins will react differently with the erythrocytes from different species based on their sugar specificity and sugar binding affinity.

### VIII. Exemplary Embodiments

Non-limiting embodiments of compositions and methods disclosed herein are as follows:

1. A method for producing a lectin in a Gram-positive bacterial cell comprising (a) obtaining a Gram-positive bacterial cell and introducing into the cell an expression cassette encoding a lectin, wherein the cassette comprises an upstream promoter sequence operably linked to a downstream open reading frame (ORF) encoding the lectin, and (b) fermenting the cell under suitable conditions for the production of the lectin.

2. The method of embodiment 1, wherein the lectin is expressed intracellularly and/or the lectin expressed and secreted extracellularly.

3. The method of embodiment 1, wherein the introduced cassette is integrated into the genome of the cell.

4. The method of embodiment 1, wherein the cell comprises at least two introduced cassettes encoding the lectin, wherein the cassettes comprise an upstream promoter sequence operably linked to a downstream ORF encoding the lectin.

5. The method of embodiment 4, wherein the at least two introduced cassettes are integrated into the genome of the cell.

6. The method of embodiment 4, wherein the at least two introduced cassettes comprise the same ORF encoding the same lectin, or comprise different open reading frames (ORFs) encoding different lectins.

7. A method for producing a lectin in a Gram-positive bacterial cell comprising (a) obtaining a Gram-positive bacterial cell and introducing into the cell an expression cassette encoding a lectin, wherein the cassette comprises an upstream promoter sequence operably linked to a downstream nucleic acid encoding a signal (secretion) sequence operably linked to a downstream open reading frame (ORF) encoding the lectin, and (b) fermenting the cell under suitable conditions for the production of the lectin.

8. The method of embodiment 7, wherein the lectin is expressed and secreted extracellularly.

9. The method of embodiment 7, wherein the introduced cassette is integrated into the genome of the cell.

10. The method of embodiment 7, wherein the cell comprises at least two introduced cassettes encoding the lectin, wherein the cassettes comprise an upstream promoter sequence operably linked to a downstream nucleic acid encoding a signal sequence operably linked to a downstream ORF encoding the lectin

11. The method of embodiment 10, wherein the at least two introduced cassettes are integrated into the genome of the cell.

12. The method of embodiment 10, wherein the at least two introduced cassettes comprise the same ORF encoding the same lectin, or comprise different open reading frames (ORFs) encoding different lectins.

13. A method for producing a lectin in a Gram-positive bacterial cell comprising (a) obtaining a Gram-positive bacterial cell and introducing into the cell a first and a second expression cassette encoding a lectin, wherein the first cassette comprises an upstream promoter sequence operably linked to a downstream open reading frame (ORF) encoding the lectin and the second cassette comprises an upstream promoter sequence operably linked to a downstream nucleic acid encoding a signal (secretion) sequence operably linked to a downstream ORF encoding the lectin, and (b) fermenting the cell under suitable conditions for the production of the lectin.

14. The method of embodiment 13, wherein the lectin is expressed intracellularly and/or the lectin is expressed and secreted extracellularly.

15. The method of embodiment 13, wherein the first introduced cassette, or the second introduced cassette, is integrated into the genome of the cell.

16. The method of embodiment 13, wherein the first and second introduced cassettes are integrated into the genome of the cell

17. The method of embodiment 13, wherein the at least two introduced cassettes comprise the same ORF encoding the same lectin, or comprise different open reading frames (ORFs) encoding different lectins.

18. The method of any one of embodiments 1, 7, or 13, wherein the ORF encodes a native lectin, or a variant lectin, derived from a cyanobacterial cell, a eukaryotic cell, or a bacterial cell.

19. The method of embodiment 18, wherein the eukaryotic cell is selected from the group consisting of plant cells, fungal cells, and animal cells.

20. The method of any one of embodiments 1, 7, or 13, wherein the ORF encodes a lectin selected from the group consisting of a native griffithsin (GRFT) lectin or a variant GRFT lectin derived therefrom, a native scytovirin (SVN) lectin or a variant SVN lectin derived therefrom, a native cyanovirin-N (CVN) lectin or a variant CVN lectin derived therefrom, a native K. alvarezii KAA-1 lectin or a variant KAA-1 lectin derived therefrom, a native K. alvarezii KAA-2 lectin or a variant KAA-2 lectin derived therefrom, a native Microcystis viridis (MVL) lectin or a variant MVL lectin derived therefrom, a native Boodlea coacta agglutinin (BCA) lectin or a variant BCA lectin derived therefrom, a native Artocarpus heterophyllus (Jacalin) lectin or a variant Jacalin lectin derived therefrom, a native Musa acuminata (Banana) lectin or a variant Banana lectin derived therefrom, a native Aaptos papilleta (Sponge) lectin or a variant Sponge lectin derived therefrom, a native Abrus precatorius (Jequirty bean) lectin or a variant Jequirty bean lectin derived therefrom, a native Aegapodium podagraria (Ground elder) lectin or a variant Ground elder lectin derived therefrom, an Agaricus bisporus (Common mushroom) lectin or a variant Common mushroom lectin derived therefrom, a native Albizzia julibrissin (Mimosa tree seed) lectin or a variant Mimosa tree seed lectin derived therefrom, a native Allomyrina dichotoma (Japanese beetle) lectin or a variant Japanese beetle lectin derived therefrom, a native Aloe arborescens (Aloe plant) lectin or a variant Aloe plant lectin derived therefrom, a native Amphicarpaea bracteata (Hog peanut) lectin or a variant Hog peanut lectin derived therefrom, a native Anguilla (Eel) lectin or a variant Eel lectin derived therefrom, a native Aplysia depilans (Mollusca) lectin or a variant Mollusca lectin derived therefrom, a native Arachis hypogaea (Peanut) lectin or a variant Peanut lectin derived therefrom, a native Bauhinia purpurea (Camel's foot tree) lectin or a variant Camel's foot tree lectin derived therefrom, a native Bryonia diocia (White bryony) lectin or a variant White bryony lectin derived therefrom, a native Caragana Arborescens (Siberian pea tree) lectin or a variant Siberian pea tree lectin derived therefrom, a native Carcinoscorpius rotundacauda (Horseshoe crab) lectin or a variant Horseshoe crab) lectin derived therefrom, a native Microcystis aeruginosa (cyanobacterium) microvirin (MVN) lectin or a variant MVN lectin derived therefrom, a native Eucheuma serra (red algae) ESA-2 lectin or a variant ESA-2 lectin derived therefrom, a native Musa acuminate (Banana) BanLec lectin or a variant BanLec lectin derived therefrom, a native Aspidistra elatior AEL lectin or a variant AEL lectin derived therefrom, a native Chaetopterus variopedatus (Marine worm) CVL lectin or a variant CVL lectin derived therefrom, a Vicia faba (Fava bean) lectin or a variant Fava bean lectin derived therefrom, a native Lens culinaris (lentil) or a variant lentil lectin derived therefrom, a native Pisum sativum (pea) lectin or a variant pea lectin derived therefrom, a jacalin-like lectin or variant jacalin-like lectin derived therefrom, including but not limited to a jacalin-like lectin set forth in SEQ ID NO: 5, SEQ ID NO: 6, SEQ ID NO: 7, SEQ ID NO: 8 and SEQ ID NO: 39, a CVN-like lectin or variant CVN-like lectin derived therefrom, including but not limited to a CVN-like lectin set forth in SEQ ID NO: 10 and SEQ ID NO: 11, a OAA-like lectin or variant OAA-like lectin derived therefrom, including but not limited to an OAA-like lectin set forth in one of SEQ ID NO: 14-SEQ ID NO: 23 or SEQ ID NO: 29-SEQ ID NO: 38, a galectin-1-like lectin or variant galectin-1-like lectin derived therefrom, including but not limited to a galectin-1-like lectin set forth in SEQ ID NO: 25-SEQ ID NO: 28, and a ricin-like lectin or variant ricin-like lectin derived therefrom, including but not limited to a ricin-like lectin set forth in SEQ ID NO: 24.

21. The method of any one of embodiments 1, 7 or 13, wherein the Gram-positive bacterial cell is selected from a member of the Firmicutes phylum.

22. The method of any one of embodiments 1, 7 or 13, wherein the Gram-positive bacterial cell is selected from a Bacillaceae family member.

23. The method of any one of embodiments 1, 7 or 13, wherein the Gram-positive bacterial cell is selected from the group consisting of Alkalibacillus sp. cells, Amphibacillus sp. cells, Anoxybacillus sp. cells, Bacillus sp. cells, Caldalkalibacillus sp. cells, Cerasilbacillus sp. cells, Exiguobacterium sp. cells, Filobacillus sp. cells, Geobacillus sp. cells, Gracilibacillus sp. cells, Halobacillus sp. cells, Halolactibacillus sp. cells, Jeotgalibacillus sp. cells, Lentibacillus sp. cells, Marinibacillus sp. cells, Oceanobacillus sp. cells, Ornithinibacillus sp. cells, Paraliobacillus sp. cells, Paucisalibacillus sp. cells, Pontibacillus sp. cells, Pontibacillus sp. cells, Saccharococcus sp. cells, Salibacillus sp. cells, Salinibacillus sp. cells, Tenuibacillus sp. cells, Thalassobacillus sp. cells, Ureibacillus sp. cells, and Virgibacillus sp. cells.

24. A method for recovering an intracellular lectin comprising (a) constructing and fermenting a recombinant cell according to any one of embodiments 1, 7, or 13. (b) lysing cells at end of the fermentation to obtain a lysed cell broth, heat treating the lysed broth at pH between 1.5 and 8.5, then cooling broth and harvesting the cooled broth, (c) subjecting the harvested cooled broth to a clarification process, and (d) subjecting the clarified broth to a concentration process, wherein the lectin is recovered in the clarified concentrated broth.

25. A method for recovering a secreted lectin comprising (a) constructing and fermenting a recombinant cell according to any one of embodiments 1, 7, or 13, and harvesting the broth, (b) subjecting the harvested broth to a clarification process, and (c) subjecting the clarified broth to a concentration process, wherein the lectin is recovered in the clarified concentrated broth.

26. A method for enhancing the purity of a lectin concentrate comprising (a) obtaining a lectin concentrate recovered according to the method of embodiment 24, or embodiment 25, adjusting the pH of concentrate to between 1.5 to 8.5, (b) incubating the concentrate of step (a) for a sufficient amount of time at a temperature between about 5° C. and 55° C., and (c) centrifuging the concentrate and collecting the supernatant, or filtering the concentrate and collecting the filtrate, wherein the collected supernatant, or the collected filtrate, comprises a substantially purified lectin relative to the purity of the lectin concentrate obtained in step (a).

A method for enhancing the purity of a lectin concentrate comprising (a) obtaining a lectin concentrate recovered according to the method of embodiment 24, or embodiment 25, adjusting the pH of the concentrate to about pH 2, incubating the concentrate at pH 2 for a sufficient amount of time at a temperature between about 55° C. and 65° C., and (b) centrifuging the incubated concentrate and collecting the supernatant, or filtering the concentrate and collecting the filtrate, wherein the collected supernatant, or the collected filtrate, comprises a substantially purified lectin relative to the purity of the lectin concentrate obtained in step (a).

28. A method for the crystallization of a lectin comprising (a) obtaining a lectin concentrate recovered according to the method of any one of embodiments 24-27, (b) adding a salt or a mixture of salts, comprising sodium, calcium, ammonium, sulfate, or chloride ions to the concentrate, adjusting the pH of the salted concentrate to about pH 2 to 5, and mixing and incubating the concentrate between about 5° C. to 50° C. for an appropriate amount of time, wherein the lectin crystallizes from the concentrate after mixing and incubating for an appropriate amount of time in step (b).

29. A method for the crystallization of a lectin comprising (a) obtaining a lectin concentrate recovered according to the method of any one of embodiments 24-27, (b) adding a salt or a mixture of salts at 0.5% to 10%, comprising sodium, calcium, ammonium, sulfate, or chloride ions to the concentrate, adjusting the pH of the salted concentrate to about pH 2 to 5, and mixing and incubating the concentrate between about 5° C. to 50° C. for an appropriate amount of time, wherein the lectin crystallizes from the concentrate after mixing and incubating for an appropriate amount of time in step (b).

30. The method of embodiment 29, wherein pH is about 2.5 to about 3.5, the salt is about 1% to about 5% sodium sulfate, and incubating temperature is about 5° C. to about 25° C.

31. The method of embodiment 29, wherein pH is about 2.8 to about 3.2, salt is about 1.8 to about 2.5% sodium sulfate, and incubating temperature is about 15° to about 25° C.

32. A method for enhancing the purity of a lectin preparation comprising (a) obtaining a lectin crystal slurry according to the method of any one of embodiments 28-31, optionally washing the crystal slurry with water and mixing, and centrifuging the slurry for sufficient amount of time, decanting, and collecting the supernatant following centrifugation, adding appropriate pH buffers to maintain pH of about 4.8 to about 8.6 with mixing at room temperature for a sufficient amount of time, and (b) filtering supernatant and collecting filtered supernatant, wherein the collected supernatant comprises a high purity lectin preparation.

A method for recovering a secreted lectin from a fermentation broth comprising (a) obtaining and harvesting a whole fermentation broth comprising a secreted lectin, (b) subjecting the harvested broth to a clarification process, and (c) subjecting the clarified broth to a concentration process, wherein the lectin is recovered in the clarified concentrated broth.

34. A method for recovering an intracellular lectin from a fermentation broth comprising (a) obtaining and lysing a whole fermentation broth comprising an intracellular lectin, heat treating the lysed broth at pH between about 1.5 and about 8.5, then cooling broth and harvesting the cooled broth, (b) subjecting the harvested cooled broth to a clarification process, and (c) subjecting the clarified broth to a concentration process, wherein the lectin is recovered in the clarified concentrated broth.

A method for enhancing the purity of a lectin concentrate comprising (a) obtaining a lectin concentrate recovered according to the method of embodiment 33, or embodiment 34, adjusting the pH of concentrate to between about 1.5 to about 8.5, (b) incubating the concentrate of step (a) for a sufficient amount of time at a temperature between about 5° C. and about 55° C., and (c) centrifuging the concentrate and collecting the supernatant, or filtering the concentrate and collecting the filtrate, wherein the collected supernatant, or the collected filtrate, comprises a substantially purified lectin relative to the purity of the lectin concentrate obtained in step (a).

36. A method for enhancing the purity of a lectin concentrate comprising (a) obtaining a lectin concentrate recovered according to the method of embodiment 33, or embodiment 34, adjusting the pH of the concentrate to about pH 2, incubating the concentrate at about pH 2 for a sufficient amount of time at a temperature between about 55° C. and about 65° C., and (b) filtering the incubated concentrate and collecting the supernatant, or filtering the concentrate and collecting the filtrate, wherein the collected supernatant, or the collected filtrate, comprises a substantially purified lectin relative to the purity of the lectin concentrate obtained in step (a).

37. A method for the crystallization of a lectin comprising (a) obtaining a lectin concentrate recovered according to the method of embodiment 35, or embodiment 36, (b) adding a salt or a mixture of salts, comprising sodium, calcium, ammonium, sulfate, or chloride ions to the concentrate, adjusting the pH of the salted concentrate to about 2 to about 5, and mixing and incubating the concentrate between about 5° C. to about 50° C. for an appropriate amount of time, wherein the lectin crystallizes from the concentrate after mixing and incubating for an appropriate amount of time in step (b).

38. A method for the crystallization of a lectin comprising (a) obtaining a lectin concentrate recovered according to the method of embodiment 35, or embodiment 36, (b) adding a salt or a mixture of salts at about 0.5% to about 10% to the concentrate, the salt or mixture of salts comprising sodium, calcium, ammonium, sulfate, or chloride ions, adjusting the pH of the salted concentrate to pH of about 2 to about 5, and mixing and incubating the concentrate between about 5° C. to about 50° C. for an appropriate amount of time, wherein the lectin crystallizes from the concentrate after mixing and incubating for an appropriate amount of time in step (b).

39. The method of embodiment 38, wherein pH is about 2.5 to about 3.5, salt is about 1% to about 5% sodium sulfate, and incubating temperature is about 5° C. to about 25° C.

40. The method of embodiment 38, wherein pH is about 2.8 to about 3.2, salt is about 1.8% to about 2.5% sodium sulfate, and incubating temperature is about 15° to about 25° C.

41. A method for enhancing the purity of a lectin preparation comprising: (a) obtaining a lectin crystal slurry according to the method of any one of embodiments 37-40, optionally washing the crystal slurry with water and mixing, and centrifuging the slurry for sufficient amount of time, decanting, and collecting the supernatant following centrifugation, adding appropriate pH buffers to maintain pH of about 4.8 to about 8.6 with mixing at room temperature for a sufficient amount of time, and (b) filtering supernatant and collecting filtered supernatant, wherein the collected supernatant comprises a high purity lectin preparation.

42. A high purity lectin preparation produced and obtained according to the methods of any one of embodiments 1-41.

43. A method for producing and recovering a high purity griffithsin (GRFT) protein preparation comprising (a) constructing a recombinant Gram-positive bacterial cell expressing a polynucleotide encoding the GRFT protein, (b) fermenting the recombinant cell for the production of the GRFT protein, lysing cells at end of the fermentation to obtain a lysed cell broth, and treating the lysed broth by holding broth for about 1 to about 4 hours at a pH of about 4.8 to about 5.2 and a temperature of about 50° C. to about 80° C., (c) clarifying the broth of step (b) by a filtration or microfiltration process, and concentrating the clarified broth by an ultrafiltration process. (d) performing a crystallization process on the concentrated broth of step (c), the crystallization process comprising adding about 2% sodium sulfate to the concentrate, adjusting pH to about 3 and incubating at about 22° C. for a sufficient amount of time, (e) centrifuging the incubated concentrate of step (d), decanting supernatant to harvest the GRFT crystal, dissolving the GRFT crystal in about 100 mM sodium acetate buffer at about pH 4.5 to about 5.5 to obtain an aqueous GRFT protein preparation, and centrifuging the GRFT preparation to remove any insoluble impurities therein and collecting the supernatant or filtering the GRFT preparation to remove any insoluble impurities therein and collecting the filtrate, wherein the supernatant collected in step (e), or filtrate collected in step (e), comprises a high purity GRFT protein preparation.

44. A method for recovering a high purity griffithsin (GRFT) preparation comprising (a) obtaining a whole fermentation broth comprising recombinant cells expressing the GRFT protein, lysing cells in the cell broth, and treating the lysed broth by holding broth for about 1 to 4 hours at a pH of about 4.8 to 5.2 and a temperature of about 50° C. to 80° C., (b) clarifying the broth of step (b) by a filtration or microfiltration process, and concentrating the clarified broth by an ultrafiltration process, (c) performing a crystallization process on the concentrated broth of step (b), the crystallization process comprising adding about 2% sodium sulfate to the concentrate adjusting pH to about 3 and incubating at about 22° C. for a sufficient amount of time, and (d) centrifuging the incubated concentrate of step (c). decanting supernatant to harvest the GRFT crystal, dissolving the GRFT crystal in about 100 mM sodium acetate buffer at about pH 4.5 to about 5.5 to obtain an aqueous GRFT protein preparation, and centrifuging the GRFT preparation to remove any insoluble impurities therein, and collecting the supernatant, or filtering the GRFT preparation to remove any insoluble impurities therein and collecting the filtrate, wherein the supernatant collected in step (d), or filtrate collected in step (d), comprises a high purity GRFT protein preparation.

45. The method of embodiment 43, or embodiment 44, wherein the high purity GRFT preparation comprises a native GRFT protein, or a variant GRFT protein preparation.

46. The method of embodiment 43 or embodiment 44, wherein the high purity GRFT preparation is at least 2.0 times higher in purity than the recovered GRFT concentrate, as determined via the GRFT concentration measured at A₂₈₀ nm.

47. The method of embodiment 43, or embodiment 47, wherein the GRFT is the major band, or the only band of about 12.7 kDa in the high purity GRFT preparation when visualized by SDS-PAGE.

48. The method of embodiment 43, or embodiment 47, wherein the high purity GRFT comprises hemagglutination activity when assayed/screened against one or more animal red blood cells (erythrocytes).

49. A protein preparation comprising the high purity GRFT produced according to the method of any one of embodiments 43-48.

50. A method for enhancing the purity of a lectin protein preparation comprising (a) obtaining a lectin fermentation broth concentrate and adjusting the pH of concentrate to about pH 2-4, adding a sufficient amount of a salt to the concentrate, centrifuging the concentrate for a sufficient amount of time, and collecting the supernatant. (b) adding an appropriate amount of a polymer to the supernatant and incubating for a sufficient amount of time to the initiate liquid-liquid extraction, and centrifuging the liquid-liquid extraction for a sufficient amount of time to precipitate the lectin protein, and (c) recovering the precipitated lectin protein pellet, wherein the recovered pellet comprises the enhanced purity lectin.

51. The method of embodiment 50, wherein the salt is selected from the group consisting of phosphates, sulfates, and citrates 52. The method of embodiment 50, wherein the salt is ammonium sulfate.

53. The method of embodiment 50, wherein the polymer is selected from the group consisting of polyethylene glycols (PEGs) of various sizes, dextrans of various sizes, chemically modified derivatives of one or more PEGs of various sizes and/or chemically modified derivatives of one or more dextrans of various sizes.

54. The method of embodiment 50, wherein the pH is about 2.

55. The method of embodiment 50, further comprising washing the recovered lectin pellet with an appropriate buffer and 30% PEG at about pH 2, followed by neutralization and solubilization of the lectin with an appropriate buffer to obtain an enhanced purity lectin protein preparation.

56. The method of embodiment 55, wherein the enhanced purity lectin preparation is at least 80% of the total protein composition.

57. The method of any one of embodiments 1, 3, or 7, wherein the recombinant cell is deficient in the production one or more endogenous (native) proteins selected from the group consisting of amylases, xylanases, pullulanases, phytases, pectate lyases, glucanases, mannosidases, lipases, esterases

58. A recombinant Gram-positive bacterial cell expressing a polynucleotide encoding a heterologous lectin.

59. The recombinant cell of embodiment 58, comprising an introduced polynucleotide cassette encoding the lectin, wherein the cassette comprises an upstream promoter operably linked to a downstream nucleic acid encoding the lectin, or wherein the cassette comprises an upstream promoter operably linked to a downstream nucleic acid encoding a signal sequence operably linked to a downstream nucleic acid encoding the lectin.

60. The recombinant cell of embodiment 58, comprising at least two introduced cassettes encoding the lectin, wherein the at least two introduced cassettes comprise an upstream promoter operably linked to a downstream nucleic acid encoding the lectin.

61. The recombinant cell of embodiment 58, comprising at least two introduced cassettes encoding the lectin, wherein the introduced cassettes comprise an upstream promoter operably linked to a downstream nucleic acid encoding a signal sequence operably linked to a downstream nucleic acid encoding the lectin.

62. The recombinant cell of embodiment 58, comprising at least two introduced cassettes encoding the lectin, wherein one introduced cassette comprises an upstream promoter operably linked to a downstream nucleic acid encoding the lectin and the second introduced cassette comprises an upstream promoter operably linked to a downstream nucleic acid encoding a signal sequence operably linked to a downstream nucleic acid encoding the lectin.

63. The recombinant cell of embodiment 58, wherein the lectin is expressed intracellularly

64. The recombinant cell of embodiment 58, wherein the lectin is expressed and secreted extracellularly.

65. The recombinant cell of embodiment 58, wherein the lectin is expressed intracellularly and expressed and secreted extracellularly.

66. The recombinant cell of embodiment 58, wherein the introduced cassette is integrated into the genome of the cell.

67. The recombinant cell of any one of embodiments 59-62, wherein at least one of the at least two introduced cassettes are integrated into the genome of the cell.

68. The recombinant cell of any one of embodiments 59-62, wherein at least two introduced cassettes are integrated into the genome of the cell.

69. The recombinant cell of any one of embodiments 59-62, wherein the at least two introduced cassettes encode the same lectin, or encode different lectins.

70. The recombinant cell of embodiment 58, wherein the Gram-positive bacterial cell is selected from a member of the Firmicutes phylum.

71. The recombinant cell of embodiment 58, wherein the Gram-positive bacterial cell is selected from a Bacillaceae family member.

72. The recombinant cell of embodiment 58, wherein the Gram-positive bacterial cell is selected from the group consisting of Alkalibacillus sp. cells, Amphibacillus sp. cells, Anoxybacillus sp. cells, Bacillus sp. cells, Caldalkalibacillus sp. cells. Cerasilbacillus sp. cells, Exiguobacterium sp. cells, Filobacillus sp. cells, Geobacillus sp. cells, Gracilibacillus sp. cells, Halobacillus sp. cells, Halolactibacillus sp. cells, Jeotgalibacillus sp. cells, Lentibacillus sp. cells, Marinibacillus sp. cells. Oceanobacillus sp. cells, Ornithinibacillus sp. cells, Paraliobacillus sp. cells, Paucisalibacillus sp. cells, Pontibacillus sp. cells, Pontibacillus sp. cells, Saccharococcus sp. cells, Salibacillus sp. cells, Salinibacillus sp. cells. Tenuibacillus sp. cells, Thalassobacillus sp. cells, Ureibacillus sp. cells, and Virgibacillus sp. cells.

73. The recombinant cell of embodiment 58, wherein the heterologous lectin is derived from a bacterial cell, a eukaryotic cell, or a cyanobacterial cell.

74. The recombinant cell of embodiment 73, wherein the eukaryotic cell is selected from the group consisting of plant cells, fungal cells, insect cells, and animal cells.

75. The recombinant cell of embodiment 58, wherein the wherein the heterologous lectin is selected from the group consisting of a native griffithsin (GRFT) lectin or a variant GRFT lectin derived therefrom, a native scytovirin (SVN) lectin or a variant SVN lectin derived therefrom, a native cyanovirin-N (CVN) lectin or a variant CVN lectin derived therefrom, a native K. alvarezii KAA-1 lectin or a variant KAA-1 lectin derived therefrom, a native K. alvarezii KAA-2 lectin or a variant KAA-2 lectin derived therefrom, a native Microcystis viridis (MVL) lectin or a variant MVL lectin derived therefrom, a native Boodlea coacta agglutinin (BCA) lectin or a variant BCA lectin derived therefrom, a native Artocarpus heterophyllus (Jacalin) lectin or a variant Jacalin lectin derived therefrom, a native Musa acuminata (Banana) lectin or a variant Banana lectin derived therefrom, a native Aaptos papilleta (Sponge) lectin or a variant Sponge lectin derived therefrom, a native Abrus precatorius (Jequirty bean) lectin or a variant Jequirty bean lectin derived therefrom, a native Aegapodium podagraria (Ground elder) lectin or a variant Ground elder lectin derived therefrom, an Agaricus bisporus (Common mushroom) lectin or a variant Common mushroom lectin derived therefrom, a native Albizzia julibrissin (Mimosa tree seed) lectin or a variant Mimosa tree seed lectin derived therefrom, a native Allomyrina dichotoma (Japanese beetle) lectin or a variant Japanese beetle lectin derived therefrom, a native Aloe arborescens (Aloe plant) lectin or a variant Aloe plant lectin derived therefrom, a native Amphicarpaea bracteata (Hog peanut) lectin or a variant Hog peanut lectin derived therefrom, a native Anguilla (Eel) lectin or a variant Eel lectin derived therefrom, a native Aplysia depilans (Mollusca) lectin or a variant Mollusca lectin derived therefrom, a native Arachis hypogaea (Peanut) lectin or a variant Peanut lectin derived therefrom, a native Bauhinia purpurea (Camel's foot tree) lectin or a variant Camel's foot tree lectin derived therefrom, a native Bryonia diocia (White bryony) lectin or a variant White bryony lectin derived therefrom, a native Caragana Arborescens (Siberian pea tree) lectin or a variant Siberian pea tree lectin derived therefrom, a native Carcinoscorpius rotundacauda (Horseshoe crab) lectin or a variant Horseshoe crab) lectin derived therefrom, a native Microcystis aeruginosa (cyanobacterium) microvirin (MVN) lectin or a variant MVN lectin derived therefrom, a native Eucheuma serra (red algae) ESA-2 lectin or a variant ESA-2 lectin derived therefrom, a native Musa acuminate (Banana) BanLec lectin or a variant BanLec lectin derived therefrom, a native Aspidistra elatior AEL lectin or a variant AEL lectin derived therefrom, a native Chaetopterus variopedatus (Marine worm) CVL lectin or a variant CVL lectin derived therefrom, a Vicia faba (Fava bean) lectin or a variant Fava bean lectin derived therefrom, a native Lens culinaris (lentil) or a variant lentil lectin derived therefrom, a native Pisum sativum (pea) lectin or a variant pea lectin derived therefrom, a jacalin-like lectin or variant jacalin-like lectin derived therefrom, including but not limited to a jacalin-like lectin set forth in SEQ ID NO: 5, SEQ ID NO: 6, SEQ ID NO: 7, SEQ ID NO: 8 and SEQ ID NO: 39, a CVN-like lectin or variant CVN-like lectin derived therefrom, including but not limited to a CVN-like lectin set forth in SEQ ID NO: 10 and SEQ ID NO: 11, a OAA-like lectin or variant OAA-like lectin derived therefrom, including but not limited to an OAA-like lectin set forth in one of SEQ ID NO: 14-SEQ ID NO: 23 or SEQ ID NO: 29-SEQ ID NO: 38, a galectin-1-like lectin or variant galectin-1-like lectin derived therefrom, including but not limited to a galectin-1-like lectin set forth in SEQ ID NO: 25-SEQ ID NO: 28, and a ricin-like lectin or variant ricin-like lectin derived therefrom, including but not limited to a ricin-like lectin set forth in SEQ ID NO: 24.

76. The recombinant cell of any one of embodiments 1, 3, or 7, wherein the recombinant cell is deficient in the production one or more endogenous (native) proteins selected from the group consisting of amylases, xylanases, pullulanases, phytases, pectate lyases, glucanases, mannosidases, lipases and esterases.

### EXAMPLES

Certain aspects of the present disclosure may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Expression of Griffithsin Variant (M78Q) in Gram-Positive Bacterial Cells

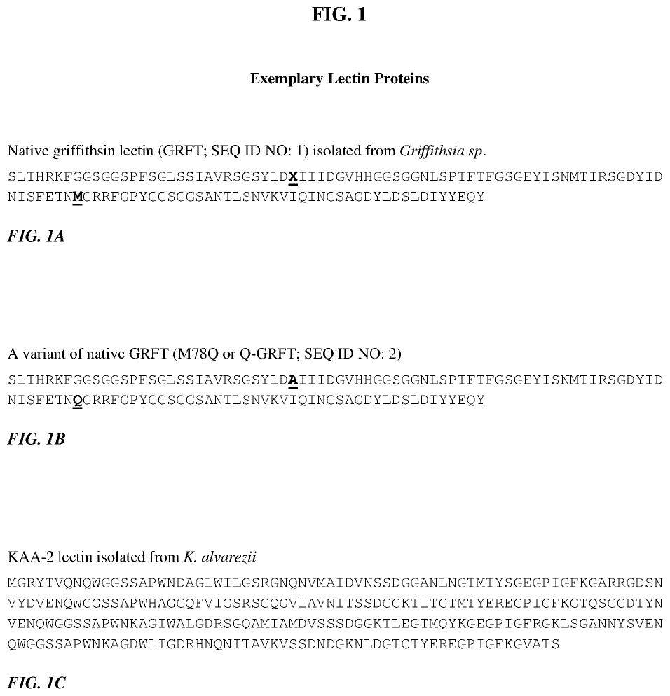

In the instant example, Applicant has designed, constructed, and evaluated exemplary Gram-positive host cells for their ability to express/produce heterologous (foreign) lectins. More particularly, as described herein, it was surprisingly observed that Gram-positive bacterial cells (e.g., Bacillus sp. cells) can express/produce significant amounts of a heterologous (eukaryotic) lectin known as griffithsin (GRFT). As set forth and exemplified in the following sections, polynucleotides (expression cassettes) encoding the Q-GRFT variant (SEQ ID NO: 2; FIG. 1B) of the native griffithsin (GRFT) protein (SEQ ID NO: 1; FIG. 1A) were constructed and evaluated in large scale bioreactors.

### Construction of Expression Cassettes and Bacillus Strains Expressing Q-GRFT

Two (2) expression cassettes encoding Q-GRFT (SEQ ID NO: 2) were constructed for intracellular expression or secreted expression of the mature Q-GRFT in Bacillus sp. cells (e.g., see schematic FIG. 6). For example, to generate expression cassettes for intracellular expression (FIG. 6A/6B), the eukaryotic derived DNA sequence (Q-grft) encoding the Q-GRFT protein was codon optimized (SEQ ID NO: 3) for expression in B. subtilis cells, and operably linked to a suitable upstream (5′) promoter (pro) sequence (e.g., 5′-[pro]-[Q-grft]-3′). To generate the expression cassettes for extracellular (secreted) expression, the codon optimized Q-grft DNA sequence (SEQ ID NO: 3) was operably linked to an upstream (5′) nucleic acid sequence encoding a (protein) signal sequence (ss) functional in the B. subtilis cells (e.g., an aprE signal sequence) which was operably linked to a suitable upstream (5′) promoter (pro) region sequence (e.g., 5′-[pro]-[ss]-[Q-grft]-3′). In addition, the alanine racemase gene (alrA) was cloned in the expression cassettes as selection marker for transformation.

The Q-grft cassette for direct secretion of Q-GRFT was used to transform exemplary B. subtilis strains herein rendered deficient in the production of one or more serine proteases and comprising a deleted alanine racemase (alrA) gene. More particularly, in the instant example, three (3) Q-GRFT production strains were generated, which include B. subtilis strains CB462, comprising deletions of the genes encoding aprE and nprE proteases; CB460, comprising deletions of the genes encoding aprE, nprE, epr, isp, bpr and wprA proteases; and CB447, comprising deletions of the genes encoding aprE, nprE, epr, isp, bpr, wprA, vpr, mpr and nprB proteases. Likewise, the Q-grft cassette for intracellular expression of Q-GRFT was used to transform a B. subtilis strain comprising deletions of the genes encoding aprE, nprE, epr, isp, bpr, wprA, vpr, mpr and nprB proteases and comprising a deleted alanine racemase (alrA) gene, which transformation resulted in the generation of B. subtilis strain CB476.

### Evaluation of Secreted Q-GRFT Produced in 10 L Bioreactors

Expression of the secreted Q-GRFT protein (SEQ ID NO: 2) in B. subtilis strains CB460, CB462 and CB447 was assessed in a 10 L bioreactor and recovered as generally described herein.

Fermentation and Recovery: The Q-GRFT production strains were grown in fermentation medium with cell growth nutrients containing carbon sources like sugars, alcohols, organic and amino acids, nitrogen sources like ammonium and nitrate salts, phosphate salts, magnesium salts, potassium and sodium salts, trace metals salts containing like iron, manganese, zinc, copper, boron, to make high amounts of cell mass. The fermentation medium was optimized for higher cell density by adding complex nutrients containing lipids, proteins, peptides, amino acids typically present in byproducts of soy, corn, yeast, processes. Cell banks of the recombinant strains were maintained in frozen state, and first inoculated into sterile fermentation medium, to prepare seed for starting a fermentor run. Seed cells were further grown in batch of nutrient salts fermentation medium, supplemented with plants derived protein hydrolysates and yeast extract as described above.

Fermentor batch is also fed with controlled amounts of sugars and ammonia, with pH controlled at optimum setpoint, and temperature controlled at optimum setpoint. Fermentation temperature was maintained at 25-45° C., in certain aspects about 37° C., by cooling water through the fermentor coil and jacket. Fermentor pH was maintained by feeding NH₃-water when pH dropped below set point at 6 to 8, (e.g., about 6.8-7.4); if necessary, phosphoric or sulfuric acid was added to maintain pH. The cell mass increased by feeding more carbon and nitrogen sources resulting in higher production of expressed protein. Nutrient salt medium was prepared in stainless steel pressure vessel fermenters, sparged with air for adequate supply of dissolved oxygen (DO). The DO is controlled at optimum setpoint. 5-50% of air saturation value. Secreted Q-GRFT is the most abundant protein in the fermentor broth after 24-48 hours, (e.g., about 36 hours). Some contaminant proteins are from the Bacillus cell debris and naturally secreted enzymes by bacilli.

In the instant example, fermentation broth was recovered to a clarified concentrate, which may be performed via a variety of methods generally starting with a broth treatment which includes lysis (e.g., natural or chemically induced lysis, natural in this example), heat treatment, pH and temperature control, water or buffer dilution, and with or without flocculation. For example, cell separation can be done in a variety of methods including, but not limited to, depth filtration or membrane-based operations. Concentration is performed via ultrafiltration membrane operations. In certain aspects, the resulting clarified concentrate is further purified as described in subsequent examples hereinafter. Additional details regarding recovery process can be found in subsequent examples and the specification of the disclosure.

More specifically, expression of Q-GRFT in the ultrafiltrate (UF) concentrates, the strained broths and the broth supernatants was evaluated via SDS-PAGE, wherein ten microliters (10 μL) of 10-fold diluted samples were evaluated by SDS-PAGE, along with the See Blue Plus 2 molecular weight standard and the lysozyme protein standard followed by staining and detaining of the gel using standard molecular biology procedures (data not shown). As indicated on the SDS-PAGE gel (data not shown), the Q-GRFT protein appears as a single band with a molecular weight between about 6 kDa and 14 kDa, wherein lane 1 is the His-tag lysozyme standard, lane 2 is the ultrafiltrate (UF) concentrated from strain CB447 (grown in a fermentation media containing 2% Soy flour, 2% Corn steep solids), lanes 3, 4 and 5 are the strained broth, supernatant broth and UF concentrated from strain CB447 (grown in a fermentation media containing 1% Corn steep solids), lanes 6, 7 and 8 are the strained broth, supernatant broth and UF concentrated from strain CB462 (grown in a fermentation media containing 1% Corn steep solids) and lanes 9, 10 and 11 are the strained broth, supernatant broth and ultrafiltrate concentrated from strain CB460 (grown in a fermentation media containing 1% Corn steep solids).

Thus, as demonstrated in the instant example, comparable Q-GRFT protein can be recovered from the fermentation broth of B. subtilis strains CB462 (with deletions of aprE, nprE), CB460 (with deletions of aprE, nprE, epr, isp, bpr, wprA), and CB447 (with deletions of aprE, nprE, epr, isp, bpr, wprA, vpr, mpr and nprB), suggesting that the Q-GRFT protein can be efficiently produced in any B. subtilis strain. More particularly, as set forth above, the use of certain strains deleted for one or more side (background) activities can be beneficial for recovery and/or purification of the Q-GRFT protein (or other lectin proteins) from the contaminant proteins and/or background enzyme activities, potentially resulting in a cleaner target protein (lectin) product.

### Evaluation of Intracellular and Secreted Q-GRFT in 10 L Bioreactors

Expression of the intracellular Q-GRFT in strain CB476 and the secreted Q-GRFT in strain CB447 were assessed in a 10 L bioreactor and recovered as generally described herein.

Fermentation and Recovery: The Q-GRFT production strains were grown in fermentation medium with cell growth nutrients containing carbon sources like sugars, alcohols, organic and amino acids, nitrogen sources like ammonium and nitrate salts, phosphate salts, magnesium salts, potassium and sodium salts, trace metals salts containing like iron, manganese, zinc, copper, boron, to make high amounts of cell mass. The fermentation medium was optimized by adding complex nutrients containing lipids, proteins, peptides, amino acids typically present in byproducts of soy, corn, yeast, processes. Cell banks of the recombinant strains were maintained in frozen state, and first inoculated into sterile fermentation medium, to prepare seed for starting a fermentor run. Seed cells were further grown in batch of nutrient salts fermentation medium, supplemented with plant protein hydrolysates and yeast extract, as described above.

Fermentor batch is also fed with controlled amounts of sugars and ammonia, with pH controlled at optimum setpoint, and temperature controlled at optimum setpoint. Fermentation temperature was maintained at 25-45° C. (e.g., about 37° C.), by cooling water through the fermentor coil and jacket. Fermentor pH was maintained by feeding NH₃-water when pH dropped below set point at 6 to 8 (e.g., about 6.8-7.4); if necessary, phosphoric, or sulfuric acid was added to maintain pH. The cell mass increased by feeding more carbon and nitrogen sources resulting in higher production of expressed protein. Nutrient salt medium was prepared in stainless steel pressure vessel fermenters, sparged with air for adequate supply of dissolved oxygen (DO). The DO is controlled at optimum setpoint. 5-50% of air saturation value. Q-GRFT is the most abundant protein in the bacilli cells after 24-48 hours (e.g., about 36 hours). In the fermentation broth, some contaminant proteins besides Q-GRFT are from the Bacillus cell debris along with naturally secreted enzymes by bacilli.

In the present example, the fermentation broth was recovered to a clarified concentrate. As generally described above, such processes can be performed via a variety of methods starting with a broth treatment which includes lysis (e.g., natural, or chemically induced lysis, natural in this example) and heat treatment, pH and temperature control, water or buffer dilution, and with or without flocculation. Likewise, cell separation can be performed in a variety of ways including depth filtration or membrane-based operation. Concentration maybe performed via ultrafiltration membrane operation. In certain embodiments, the resulting clarified concentrate is further purified as described in subsequent examples hereinafter.

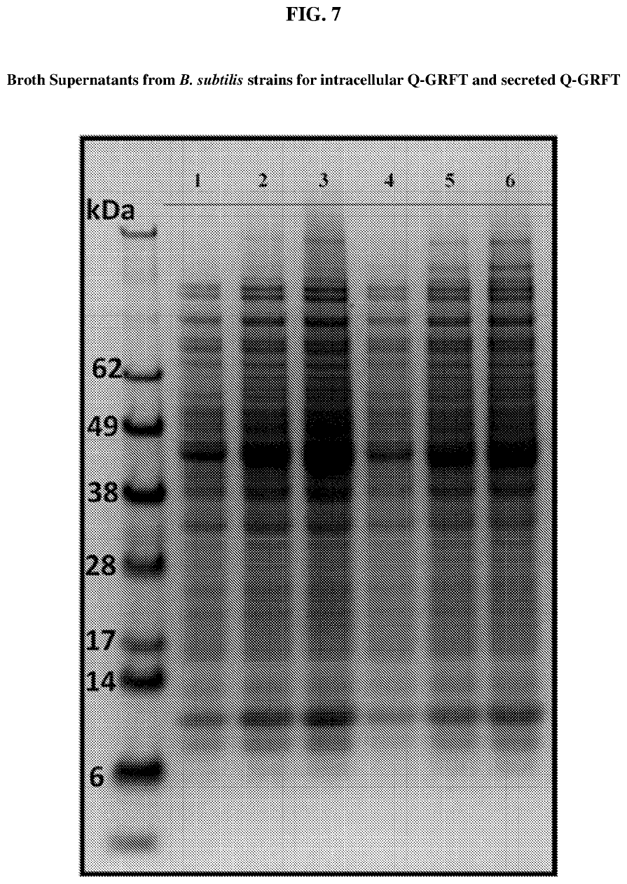

More particularly, the expression of Q-GRFT in the broth supernatants from B. subtilis strains CB447 and CB476 was evaluated via SDS-PAGE. As presented in FIG. 7, ten (10) μL of 10-fold diluted samples (lanes 1-6), along with the See Blue Plus 2 molecular weight standard (labeled, “kDa”) and the lysozyme protein standard (MClab), followed by staining and detaining of the gel using standard molecular biology procedures. As shown in FIG. 7, lanes 1, 2 and 3 are the broth supernatants from strain CB447 at eighteen (18) hours, twenty-four (24) hours and thirty (30) hours of fermentation, respectively, and lanes 4, 5 and 6 are the broth supernatants of strain CB476 at eighteen (18) hours, twenty-four (24) hours and thirty (30) hours of fermentation, respectively, wherein the Q-GRFT protein appears as a single band with the molecular weight of 12.7 kDa.

Thus, as described above, the exemplary Gram positive (B. subtilis) strains CB447, with secreted Q-GRFT expression/production and CB476, with intracellular Q-GRFT expression, produced comparable amounts of Q-GRFT under the large-scale conditions tested. More particularly, the instant example demonstrates that Gram positive bacterial cells/strains (e.g., Bacillus sp.) are particularly useful host strains for large scale fermentation and production of lectin proteins.

### Example 2

Clarified Broth Concentrate from Untreated Broth

In the instant example, fermentation broth from the above-described B. subtilis strain CB447 (i.e., expressing/secreting Q-GRFT; SEQ ID NO: 2) was processed according to the Materials and Methods described herein. More specifically, the materials, compositions and/or methods set forth in Examples 2-14 are not meant to be limiting, as one skilled in the art may readily modify, adjust, refine, and the like such Gram-positive bacterial strains, materials, and/or and methods to suit the particular requirements of any given lectin protein of the disclosure.

### Materials

Materials included the following: (i) fermentation broth from strain CB447 (Example 1), (ii) 180 μm mesh screen, microfilter fitted with 0.1 μm 80 mil spacer 4″ diameter, 17″ length spiral wound membrane, (iii) ultrafilter fitter with C-spacer cartridge 5K molecular weight cutoff (MWCO), sulfuric acid and (iv) SDS-PAGE system and reagents. In the instant example, SDS-PAGE system was 4-12% Bis-Tris reducing and MES running buffer.

### Methods and Results

The CB447 fermentation broth was generally strained through a 180 μm screen, wherein the strained (screened) broth was subsequently diluted with 1.1 parts DI water. The diluted broth was transferred to a microfilter feed tank, and a microfiltration process performed by operating at constant feed tank level diafiltration with 4× feed volumes of DI water pH 7.4 (adjusted with sulfuric acid) at 10° C., wherein the permeate was collected. The collected microfiltration permeate was concentrated using 5K MWCO membrane to an 8× initial broth volume. The Q-GRFT recovery yield was 65%, wherein a visually clear concentrate was produced (data not shown), and the obtained concentrate purity was higher than the broth supernatant by SGS-PAGE.

### Example 3

### Purification of Clarified Broth Concentrate by pH Adjustment

In the instant example, the clarified broth concentrate recovered in Example 2 was further purified by pH adjustment, according to the Materials and Methods described herein.

### Materials

Materials included the following: (i) clarified broth UF concentrate from Example 2, (ii) sulfuric acid, (iii) sodium hydroxide, (iv) pH meter, (v) shaker with temperature control, (vi) centrifuge and (vii) SDS-PAGE system and reagents.

### Methods and Results

Adjust 100 mL of UF concentrate prepared in Example 2 to pH 2 using sulfuric acid.

Readjust (raise) pH back up with sodium hydroxide, taking approximately 25 mL samples during adjustment when reaching pH 5.5, pH 6.8 and pH 8. The pH adjusted samples were then each divided into three (3) portions for incubation at 5° C., 22° C., and 55° C. for twenty-four (24) hours. Centrifuge the incubated samples at 14000 rpm for ten (10) minutes, and collect supernatants for analysis by SDS-PAGE.

All pH treated supernatants have higher purity than initial whole broth sample (data not shown).

### Example 4

### Purification of Clarified Broth Concentrate by Low pH Treatment

In the instant example, the clarified broth concentrate recovered in Example 2 was further purified by low pH treatment (i.e., pH 2) according to the Materials and Methods described herein.

### Materials

Materials include the following: (i) clarified broth concentrate from Example 2, (ii) sulfuric acid, (iii) activated carbon, (iii) pH meter. (iv) shaker with temperature control, (v) benchtop Nalgene 0.2 mm filter and (vi) SDS-PAGE system and reagents.

### Methods and Results

Adjust 150 mL of clarified broth concentrate (Example 2) to pH 2 using sulfuric acid, and split the pH adjusted sample into two portions. To one of the portions, add 1% activated carbon, check pH and adjust to maintain pH 2 as necessary. Take a time zero (TO) sample of the two portions. Split each portion into three tubes for incubation at 5° C., 22° C. and 60° C. for eighteen (18) hours. Filter incubated samples, collect filtrates, and analyze filtrate for purity by SDS-PAGE.

In particular, significant purification of Q-GRFT was achieved with pH adjustment to 2 followed by filtration, wherein sample incubation at 60° C. further improved protein purity (data not shown), and the addition of activated carbon further reduced color of the filtrate (data not shown).

### Example 5

Clarified Broth Concentrate from pH 5-5.5 and Heat-Treated Broth Recovered Using Microfilter for Cell Separation

In the instant example, fermentation broth from the above-described B. subtilis strain CB488 (i.e., Q-GRFT intracellular and extracellular expression; SEQ ID NO: 2) was processed according to the Materials and Methods described herein.

### Materials

Materials included the following: (i) fermentation broth from strain CB488 expressing Q-Grft, (ii) sulfuric acid, (iii) lysozyme, 20% Stock (BioSeutica), (iv) 180 mm mesh screen, (v) microfilter fitted with 0.1 mm 80 mmil spacer 4″ diameter, 17″ length spiral wound membrane. (vi) ultrafilter fitter with C-spacer cartridge 5K MWCO, and (vii) SDS-PAGE system and reagents. In the instant example, SDS-PAGE system was 4-12% Bis-Tris reducing SDS-PAGE, MES running buffer.

### Methods and Results

In the present method, a lysozyme treatment was performed at end of fermentation, by adding 1.5 mL of 20% stock lysozyme per L broth, and allowed to mix at 37° C. for four (4) hours. The pH was adjusted to 5.0-5.5 using sulfuric acid. Heat treatment was performed by heating tank to 60° C. and holding for four (4) hours. The pH adjusted and heat-treated broth was harvested by cooling the broth to 15° C. and transferring into a container. The harvested broth was recovered using the same microfiltration and ultrafiltration procedures outlined and described in Example 2.

A visually clear concentrate was produced. For example, the heat-treated broth supernatant provided a significant purity improvement over untreated broth supernatant (data not shown). Likewise, the Q-GRFT purity remained through the processing steps, e.g., microfiltration for cell separation and ultrafiltration for dewatering, wherein the obtained UF concentrate has higher purity than untreated broth derived UF concentrate in Example 2, recovered using similar process.

### Example 6

Clarified Broth Concentrate from pH 5-5.5 and Heat-Treated Broth Recovered Using Buchner Filter for Cell Separation

In the instant example, fermentation broth from B. subtilis strain CZ438 was processed according to the Materials and Methods described herein.

As generally described herein, the CZ438 cell comprises two (2) introduced (transformed) copies of a Q-grft expression cassette integrated into the aprE locus (1^{st} copy) and the skfABCEFGH locus (2^{nd} copy) of the B. subtills cell. For example, the Q-grft cassette comprises an upstream (5′) DNA sequence (aprE) encoding an AprE signal sequence (i.e., for secretion of mature Q-GRFT) operably linked to a downstream (3′) DNA sequence (Q-grft) encoding the Q-GRFT protein. In the present example, the B. subtills strain comprises a deleted alanine racemase (alrA) gene used for selection.

### Materials

Materials included the following: (i) fermentation broth from strain CZ438 expressing Q-GRFT, (ii) sulfuric acid, (iii) lysozyme, 20% Stock (BioSeutica), (iv) 781G floc polymer, (v) FW12 diatomaceous earth, (vi) Buchner filter fitted with HR900 pad, (vii) ultrafilter fitted with C-spacer cartridge 5K MWCO, and (viii) SDS-PAGE system and reagents. In the instant example, SDS-PAGE system was 4-12% Bis-Tris reducing SDS-PAGE, MES running buffer.

### Methods and Results

The fermentation broth from strain CZ438 was processed using the same lysozyme treatment, pH adjustment and heat treatment processes described above in Example 5. In the instant example, the harvested broth was recovered as follows. Add an equal part of DI water to harvested broth and adjust pH to maintain pH 5.5 as needed. Add 0.2% 781G Floc Polymer (as 20% solution) and mx for five (5) minutes, then add 7.5% FW12 and mix well. Filter through Buchner filter fitted with HR900 filter pad by vacuum, and collect filtrate. The collected filtrate was concentrated using an ultrafilter.

A visually clear concentrate was produced (FIG. 9), wherein the pH adjusted, and heat-treated broth supernatants (FIG. 9, lanes 3 and 4) demonstrated significant purity improvements over untreated broth supernatants (FIG. 9, lane 1). The Q-GRFT purity remained through the processing steps, flocculation and Buchner filtration for cell separation and ultrafiltration for dewatering. The obtained UF concentrate (FIG. 9, lane 7) has purity similar to that of Example 5, derived from the CB488 strain broth similarly treated, but recovered using microfiltration for cell separation; and higher purity than untreated broth derived UF concentrate (Example 2).

### Example 7

Clarified Broth Concentrate from pH 4.8-5.2 and Heat-Treated Broth Recovered Using Buchner Filter for Cell Separation

In the instant example, fermentation broth from the B. subtilis strain CB447 described in Example 2 was processed according to the Materials and Methods described herein.

### Materials

Materials included the following: (i) fermentation broth from strain CB488 expressing Q-GRFT, (ii) sulfuric acid, (iii) 781G floc polymer, (iv) FW12 diatomaceous earth. (v) Buchner filter fitted with HR900 vpad, (vi) ultrafilter fitted with C-spacer cartridge 5K MWCO, and (vii) SDS-PAGE system and reagents. In the instant example, SDS-PAGE system was 4-12% Bis-Tris reducing SDS-PAGE, MES running buffer.

### Methods and Results

In the instant method, an end of fermentation hold was performed as follows. Stop glucose and ammonia feeds, reduce air flow, and hold broth at 37° C. for four (4) hours with gentle mixing. Adjust pH to 4.8-5.2 using sulfuric acid. Heat Treatment: Heat tank to 60° C. and hold for four (4) hours. Broth Harvest: Cool broth to 15° C. and transfer into container. Recovery Procedure: Same as outlined in Example 6. A visually clear concentrate was produced, wherein the pH adjusted and heat-treated broth supernatant provided significant purity improvements over untreated broth supernatant (data not shown). Likewise, the Q-GRFT purity remained through the processing steps, flocculation and Buchner filtration for cell separation and ultrafiltration for dewatering. In particular, the obtained UF concentrate had the highest purity improvement when compared to UF concentrates obtained from broth, with different broth treatments (e.g., see Example 2, no heat treatment; and Example 5, with pH 5-5.5 and heat treatment).

### Example 8

### Crystallization of Clarified Broth Concentrates Using pH 3, 2% Sodium Sulfate

In the instant example, clarified UF concentrates from Examples 2, 5, 6 and 7 were processed according to the Materials and Methods described herein.

### Materials

Materials included the following: (i) clarified UF concentrates from Example 2, Example 5, Example 6 and Example 7, (ii) sodium sulfate, (iii) sulfuric acid and (iv) microscope.

### Methods and Results

In the instant example, the clarified concentrates were processed as follows. Add 2% sodium sulfate to clarified broth concentrate, adjust the pH to 3, incubate at 22° C. with mixing and monitor morphology over time. More specifically, all of the concentrates processed according to such methods gave square to rectangular plate shaped crystals (data not shown).

### Example 9

Crystallization of pH 2 Treated Clarified Broth Concentrate Filtrates with Sodium Sulfate

In the instant example, pH 2 treated filtrates from Example 4 were processed according to the Materials and Methods described herein.

### Materials

Materials included the following: (i) pH 2 treated filtrates from Example 4, (ii) 10% sodium sulfate, (iii) sodium hydroxide, (iv) pH meter, (v) shaker with temperature control and (vi) microscope and supplies.

### Methods and Results

In the instant example, the pH 2 treated filtrates were processed as follows. For crystallization, adjust filtrate from pH 2 to pH 3, using sodium hydroxide. Add 10% sodium sulfate stock solution to reach 2% sodium sulfate in the prepared solution, check pH (and adjust if pH is not in range of 2.8-3.2) and incubate at 22° C. with mixing. Monitor morphology by microscope observation. As generally summarized below in TABLE 1, crystals form in all of the filtrates processed according to these methods.

TABLE 1
Q-GRFT PROTEIN CRYSTALS
Pretreatment Condition

| | Activated | Time to Crystal | |
|---|---|---|---|
| Temp (° C.) | Carbon | Formation | Morphology |
| 5 | 0% | 0-19 hours | Square Plates |
| 22 | 0% | 19-40 hours | Square & Rectangular Plates |
| 60 | 0% | 40-100 hours | Long Rectangular Plates |
| 5 | 1% | 19-40 hours | Square & Rectangular Plates |
| 22 | 1% | 0-19 hours | Square Plates |
| 60 | 1% | 19-40 hours | Needles |

### Example 10

Crystallization of pH 2 Treated Clarified Broth Concentrate Filtrates with Sodium Sulfate

In the instant example, pH 2 incubated filtrates from Example 4 were processed according to the Materials and Methods described herein.

### Materials, Methods and Results

Materials included the following: pH 2 (5° C.) and pH 2 (60° C.) incubated filtrates from Example 4. The filtrates were prepared using procedures similar to those outlined in Example 4, except adjustment to pH 3 and incubated at 5° C. and 60° C. The adjusted filtrate samples are held at 5° C. and the morphology monitored over time, wherein square to rectangular plate shaped crystal formed in all the filtrates after forty-eight (48) hours at 5° C. (data not shown).

### Example 11

### Crystal Recovery and Crystal Pellet Dissolution

The instant example describes recovery of crystals and crystal pellet dissolution according to the Materials and Methods described herein.

### Materials

Materials included the following: (i) crystal slurry derived from Example 2 clarified broth concentrate (see Example 7), (ii) centrifuge, (iii) 100 mM Tris Buffer (pH 8.6), (iv) 100 mM Bis Tris (pH 6.5), (v) 100 mM sodium acetate (pH 5), (vi) 0.2 μm Nalgene bench top filter and (vii) SDS-PAGE system and reagents.

### Methods

### A. No Wash Crystal Recovery

(1) After forty-seven (47) hours of crystallization, aliquot 20 g crystal slurry into 3 centrifuge tubes, (2) centrifuge at 3000 rpm for 15 minutes, (3) decant supernatant, (4) add the following buffer to each tube to reach initial weight of 20 g: 100 mM Tris Buffer (pH 8.6), 100 mM Bis Tris (pH 6.5) and 100 mM sodium acetate (pH 5), (5) let mix at room temperature for one (1) hour and (6) filter through 0.2 μm Nalgene bench top filter.

### B. 1× Wash Crystal Recovery

Same process as above, except in step (1), add 20 g of DI water and mix.

### Results

Crystallization yield=92%

TABLE 2 shows the ratios of impurities relative to lectin protein at an absorbance wavelength of 280 nm (A₂₈₀) for dissolved crystal pellet filtrates, wherein the numbers in brackets are purification factor over initial clarified broth concentrate. As presented in TABLE 2, all the values are lower than the initial clarified broth concentrate ratio of 4.96, wherein the obtained lectin preparations were at least 2.6 to 6.8 times higher in purity.

TABLE 2
IMPURITIES TO LECTIN A₂₈₀ RATIOS OF FILTRATES FROM
NO WASH AND 1X WASH CRYSTAL PELLET DISSOLUTION

| Buffer | No Wash | 1xWash |
|---|---|---|
| pH 8.6 Tris | 1.89 (2.6) | 1.55 (3.2) |
| pH 6.5 Bis-Tris | 1.18 (4.2) | 0.79 (6.3) |
| pH 5 Sodium Acetate | 1.13 (4.4) | 0.67 (6.8) |

More particularly, as presented in FIG. 8, high purity protein preparations were obtained by dissolving crystal pellets in 100 mM sodium acetate (pH 5.5), followed by filtration. For example, the above-described TABLE 2 buffers (i.e., 100 mM Tris Buffer, pH 8.6; 100 mM Bis Tris, pH 6.5; or 100 mM sodium acetate, pH 5), are represented in FIG. 8 in lanes 3, 6, 10, 13 (100 mM Tris Buffer. pH 8.6), lanes 4, 7, 11, 14 (100 mM Bis-Tris, pH 6.5) and lanes 5, 8, 12, 15 (100 mM sodium acetate, pH 5.5).

As shown in FIG. 8, Q-GRFT is the only band present on SDS-PAGE in both the no wash and 1× wash dissolved filtrates. Additionally, all filtrates were very clear, wherein the darkest colors were observed with pH 8.6 dissolution and the pH 5 filtrates were very light in color (data not shown).

### Example 12

Crystal Recovery and Pellet Dissolution from Crystal Slurries Obtained from Various Strains Harvested and Recovered Herein

The instant example describes recovery of crystals and crystal pellet dissolution according to the Materials and Methods described herein.

### Materials

Materials included the following: (i) crystal slurry prepared from Example 4 clarified broth concentrate (see Example 7), (ii) crystal slurry prepared from Example 5 clarified broth concentrate (Example 7), (iii) crystal slurry prepared from Example 6 clarified broth concentrate (Example 7), (iv) crystal slurry prepared from Example 2 filtrate using procedure (Example 7), (v) centrifuge, (vi) 100 mM Tris Buffer (pH 8.6), (vii) 100 mM Bis Tris (pH 6.5), (viii) 100 mM sodium acetate (pH 5), (ix) 0.2 μm Nalgene bench top filter and (x) SDS-PAGE system and reagents.

### Methods

### A. No Wash Crystal Recovery

(1) After forty-seven (47) hours of crystallization, aliquot 20 g crystal slurry into 3 centrifuge tubes, (2) centrifuge at 3000 rpm for 15 minutes, (3) decant supernatant, (4) add the following buffer to each tube to reach initial weight of 20 g: 100 mM Tris Buffer (pH 8.6), 100 mM Bis Tris (pH 6.5) and 100 mM sodium acetate (pH 5), (5) let mix at room temperature for one (1) hour and (6) filter through 0.2 μm Nalgene bench top filter.

### B. 1× Wash Crystal Recovery

Same process as above, except in step (1), add 20 g of DI water and mix.

### Results

A single band or near single band purity filtrates were obtained from crystal slurries derived from the various strains, harvested, and recovered according to processes set forth above in the Examples (data not shown).

### Example 13

### Crystallization of Clarified Broth Concentrate by Varying pH, Temperature and Salt Addition

The instant example describes crystallization of the clarified broth concentrate recovered in Example 8, according to the Materials and Methods described herein.

### Materials

Materials included the following: (i) clarified broth concentrate prepared using procedure described in Example 6, (ii) sodium sulfate, (iii) ammonium sulfate, (iv) sodium chloride, (v) calcium chloride, (vi) sulfuric Acid and (vii) microscope.

### Methods and Results

For each salt to be tested, add 2% of salt to clarified broth concentrate, divide into three (3) portions. Adjust the pH of one portion to pH 4.2, another portion to pH 3 and another portion to pH2 using sulfuric acid; and divide each of the pH adjusted portions (i.e., pH 4.2, 3 and 2) for incubation with mixing at 10° C. 22° C. and 50° C., wherein morphology is monitored over time via microscope. Same tests were set up for conditions without salt addition. Between about four (4) to ninety (90) hours, plate shaped crystals ranging from square, rectangular to diamond formed.

More particularly, crystals formed in the solutions without salt addition at about pH 2 at 10° C., 22° C. and 50° C., as well as pH 3 at 50° C. Crystals also formed in the solutions with sodium sulfate or ammonium sulfate, at pH 2, pH 3 and pH 4.2 at 50° C., as well as pH 2 and pH 3 at 20° C. and 22° C. Likewise, crystals formed in the solutions with sodium chloride or calcium chloride, pH 2 at 10° C., 22° C. and 50° C. as well as pH 4.2 at 22° C. and 50° C.

### Example 14

Methods to Produce High-Purity Griffithsin Preparations from a Fermentation Broth

The instant example describes methods to recover lectins such as Q-GRFT, in substantially pure form from a fermentation broth in which recombinant host cells (e.g., bacterial cells, plant cells, insect cells, and the like) have been fermented. More particularly, such exemplary purification methods provide combinations of water-soluble polymers and salts, that spontaneously separate into two (2) fractions, one of which fractions comprises the lectin. Such methods may be referred to as a liquid-liquid extraction (or a two-phase extraction), wherein the target substance (lectin) preferentially partitions into one of the fractions. Thus, the materials and methods described herein are not meant to be limiting, as one skilled in the art may readily modify, adjust, refine and the like such materials and methods to suit the particular requirements of any given lectin protein of the disclosure. For example, commonly used water-soluble polymers for such liquid-liquid (two-phase) extractions include, but are not limited to, polyethylene glycols (PEGs) of various sizes, dextrans of various sizes, and chemically modified derivatives thereof. Commonly used salts include, but are not limited to, phosphates, sulfates, and citrates. More particularly, the combinations and concentrations of the salts and polymers are adjusted for optimal recovery and purity of the target lectin.

In particular, as exemplified herein, the purification of the lectin (Q-GRFT) from a heterologous host was greatly aided by low pH treatment. In the instant case, a pH of about pH 2-4 was most useful for separating host proteins from the target lectin (Q-GRFT). For example, in certain aspects, the liquid-liquid (two-phase) separation can be carried out by centrifugation, or in some instances, separation may occur spontaneously over the course of several hours. In addition, the liquid-liquid (two-phase) extraction method is especially efficient when carried out at low pH, resulting in superior recovery and purity compared to the same extraction at neutral pH. Thus, a particular benefit of the instant liquid-liquid extraction methods is that there is no need to utilize a time-consuming and costly chromatography step.

More particularly, in the instant example, a concentrated clarified fermentation broth was made 12% (w/w) ammonium sulfate and adjusted to pH 2 by the addition of hydrochloric acid (HCl) or sulfuric acid (H₂SO₄). The precipitated material (impurities) was separated by centrifugation, and the supernatant was made 30% (w/v) polyethylene glycol (PEG) 3350 to initiate the liquid-liquid extraction. After incubation overnight at 4° C., the material was centrifuged, and the precipitated pellet material was recovered. The recovered pellet was washed once (1×) with 20 mM glycine (pH 2), 30% PEG, followed by neutralization and solubilization by the addition of 40 mM Tris base. As presented in FIG. 9, the lectin (Q-GRFT) preparations obtained/recovered were greater than 90% pure.

### Example 15

### Expression of Jacalin-Like, CVN-Like, OAA-Like and Galectin-1 Lectins in Gram+ Bacterial Cells

In the instant example, Applicant has designed, constructed, and evaluated exemplary Gram-positive host cells for their ability to express/produce heterologous (foreign) lectins. More particularly, as described herein, it was surprisingly observed that Gram-positive bacterial cells can express/produce significant amounts of heterologous lectins known as jacalin-like, CVN-like, OAA-like. Ricin-like, and galectin-1-like lectins. As set forth and exemplified in the following sections, polynucleotides (expression cassettes) encoding the jacalin-like. CVN-like. OAA-like, Ricin-like, and galectin-1-like lectins were constructed and evaluated in 2.5 L Ultra-yield flask.

### Construction of Expression Plasmids and Bacillus Strains Expressing Jacalin-Like, CVN-Like, OAA-Like, Ricin-Like, and Galectin-1-Like Lectins

Thirty-five (35) expression plasmids encoding the jacalin-like, CVN-like, OAA-like, Ricin-like, and galectin-1-like lectins (TABLE 3) were constructed for expression of the mature lectins in a B. subtilis host. More particularly, in the instant example, thirty-five (35) lectin production strains were generated, which were based on the B. subtilis strain CBS12. For instance, the gene (ORF) coding lectin sequences (i.e., without a secretion (signal) sequence) were codon-optimized based on B. subtilis codon preference and cloned into the SpeI and HindIII restriction enzyme in the p3JM vector (FIG. 10) using double digestion and ligation method. The plasmid contained a B. subtilis aprE promoter operably linked to a codon-optimized nucleotide sequence (ORF) encoding the lectin protein and chloramphenicol acetyltransferase (CAT) as selection marker for transformation. The constructed vector was subjected to rolling-circle amplification and transformed into the host B. subtilis CBS12 and plated on Luria Agar plates supplemented with 5 ppm chloramphenicol. Thus, in certain in or more embodiments, Gram-positive host cells comprising introduced expression vectors/cassettes (e.g., p3JM) encoding lectin proteins without the use of a secretion sequence can express and retain the lectins intracellularly and/or secrete the lectins extracellularly (e.g., into the broth via a general secretory pathway) when fermented under suitable conditions for the expression/production of the lectins.

### Evaluation of Secreted Lectins Produced in Ultra-Yield Flask

Expression of the lectin proteins in B. subtilis strain CBS12 were assessed in a 2.5 L ultra-yield flask and recovered as generally described herein. More particularly, characterization of the lectins expressed in the recombinant Bacillus host cells are summarized below in TABLE 3 (FIG. 11). In particular, certain OAA-like lectins (e.g., SEQ ID NO: 14, SEQ ID NO: 18-20) described herein (TABLE 3) were expressed in similar amounts in the cell broth and the cell lysate (i.e., when the cell pellet was lysed), indicating low secretion efficiency of certain lectins without the use of a signal (secretion) sequence.

TABLE 3
CHARACTERIZATION OF LECTINS EXPRESSED IN GRAM-
POSITIVE HOSTS CELLS OF THE DISCLOSURE

| | | | | | NCBI | | |
|---|---|---|---|---|---|---|---|
| | Common | SEQ ID | | Source | Taxonomy | Expression | |
| Lectin Class | Name | NO | Fold | Species | Type | Level | Purified |
| Jacalin-like | Banlec | 5 | β-prism | Musa acuminate | Plant | ++ | Y |
| | | 6 | β-prism | Helianthus annuus | Plant | + | N |
| | AYR06195 | 7 | β-prism | Renouxia sp. | Plant | ++ | N |
| | Jacalin | 8 | β-prism | Artocarpus integrifolia | Plant | ++ | Y |
| | | 39 | | Musa troglodytarum | plant | +++ | N |
| CVN-like | MVL | 10 | β-sandwich | Microcystis viridis | cyanobacteria | +++ | Y |
| | MVN | 11 | β-sandwich | Microcystis | cyanobacteria | + | Y |
| | | | | aeruginosa PCC7806 | | | |
| OAA-like | EDA2 | 14 | β-barrel | Eucheuma | red algae | ++ | Y |
| | | | | denticulatum | | | |
| | MPA-2 | 15 | β-barrel | Meristotheca papulosa | red algae | + | Y |
| | OAA | 16 | β-barrel | Oscillatoria agardhii | cyanobacteria | ++ | Y |
| | | | | NIES-204 | | | |
| | ESA-2 | 17 | β-barrel | Eucheuma serra | red algae | + | Y |
| | | 18 | β-barrel | Herpetosiphon | bacteria | ++ | N |
| | | | | aurantiacus DSM 785 | | | |
| | | 19 | β-barrel | Roseofilum | cyanobacteria | ++ | Y |
| | | | | reptotaenium AO1-C | | | |
| | | 20 | β-barrel | Pseudomonas baetica | bacteria | + | Y |
| | | 21 | β-barrel | Verrucomicrobiaceae | bacteria | + | Y |
| | | | | bacterium | | | |
| | | 22 | β-barrel | Melittangium boletus | bacteria | + | Y |
| OAA-like | | 23 | β-barrel | Rhodocyclaceae | bacteria | ++ | Y |
| | | | | bacterium | | | |
| | | 29 | | Trichodesmium sp. | bacteria | +++ | N |
| | | | | ALOHA_ZT_67 | | | |
| | | 30 | | Burkholderia | bacteria | ++ | N |
| | | | | ubonensis | | | |
| | | 31 | | Aquimarina longa | bacteria | ++ | N |
| | | 32 | | Microcystis | bacteria | +++ | N |
| | | | | aeruginosa | | | |
| | | 33 | | Corallococcus sp. | bacteria | +++ | N |
| | | | | Z5C101001 | | | |
| | | 34 | | Agardhiella subulata | fish | +++ | N |
| | | 35 | | Sphingomonas sp. | bacteria | + | N |
| | | | | TDK1 | | | |
| | | 36 | | Proteobacteria | bacteria | ++ | N |
| | | | | bacterium | | | |
| | | 37 | | Sinobacterium | bacteria | ++ | N |
| | | | | caligoides | | | |
| | | 38 | | Aquimarina sp. TRL1 | bacteria | +++ | N |
| | Actinohivin | 24 | β-trefoil | Longispora albida | bacteria | ++ | Y |
| Ricin-like | | 25 | β-sandwich | Mus musculus | mammalian | + | Y |
| Galectin-like | Galectin-1 | 26 | β-sandwich | Desmodus rotundus | mammalian | + | Y |
| | Galectin-1 | 27 | β-sandwich | Scleropages formosus | fish | + | Y |
| | Galectin-1 | 28 | β-sandwich | Callorhinchus milii | fish | ++ | Y |

Y: purified; N: not purified.

### Fermentation and Recovery

As presented above in TABLE 3, the recombinant host cells expressing lectin proteins were grown in fermentation medium (Grant's II medium) with cell growth nutrients containing carbon sources like sugars, alcohols, organic and amino acids, nitrogen sources like ammonium and nitrate salts, phosphate salts, magnesium salts, potassium and sodium salts, trace metals salts containing like iron, manganese, zinc, copper, cobalt, molybdate, calcium, boron, to make high amounts of cell mass. Cell banks of the recombinant strains (cells) were maintained in frozen state, and first inoculated into sterile LB medium, to prepare seed for ultra-yield flask fermentation. Seed cells were further grown in batch of fermentation medium. The strains were cultured in 2.5 L flask in Infos shaker with 220 rpm rotation. Fermentation temperature was maintained at 37° C. for 16 hours, then lowered to 32° C. and cultured for another 32 hours. Some contaminant proteins are from the Bacillus (host) cell debris and naturally secreted host cell enzymes.

In the instant example, fermentation broth was recovered to a clarified concentrate, which may be performed via a variety of methods generally starting with a broth treatment which includes lysis (e.g., natural, or chemically induced lysis, natural in this example), heat treatment, pH and temperature control, water or buffer dilution, and with or without flocculation. For example, cell separation can be done in a variety of methods including, but not limited to, centrifugation, depth filtration or membrane-based operations. Concentration is performed via ultrafiltration membrane operations. In certain aspects, the resulting clarified concentrate is further purified as described in subsequent examples hereinafter. Additional details regarding the recovery process can be found in subsequent examples and the specification of the disclosure.

For example, expression of the jacalin-like (FIG. 2), CVN-like (FIG. 3), OAA-like (FIG. 4), Ricin-like (FIG. 5), and galectin-1-like (FIG. 5) lectins in the broth supernatants was evaluated via SDS-PAGE (data not shown), wherein fifteen microliters (15 μL) of samples were evaluated by SDS-PAGE, along with the Invitrogen broad spectrum molecular weight standard or the Sangon RealBand protein standard followed by staining and detaining of the gel using standard molecular biology procedures. In particular, the jacalin-like proteins (SEQ ID NO: 5, SEQ ID NO: 6, SEQ ID NO: 7, SEQ ID NO: 8 and SEQ ID NO: 39) appeared on the SDS-PAGE as a single band with molecular weights between about 10 kDa and 31 kDa. In certain aspects, it was observed that the molecular weight of jacalin-like lectin of SEQ ID NO: 39 (FIG. 2E) is about 31 kDa (data not shown), which is double of its theoretical molecular weight, which may be caused by strong binding of two monomers.

Likewise, the CVN-like proteins (FIG. 3; SEQ ID NO: 10 and SEQ ID NO: 11;) appeared on the SDS-PAGE as a single band with molecular weight between about 10 kDa and 15 kDa (data not shown), the OAA-like proteins (FIG. 4; SEQ ID NOS: 14-23, and SEQ ID NOS: 29, 30, 31, 32, 33, 34, 35, 36, 37, 38) appeared on the SDS-PAGE as a single band with molecular weight between about 10 kDa and 40 kDa (data not shown). The ricin-like lectin (FIG. 5: SEQ ID NO: 24) appeared as a single band on the SDS-PAGE with molecular weight between about 10 kDa and 40 kDa, and the galectin-like proteins (FIG. 5; SEQ ID NO: 25, SEQ ID NO: 26, SEQ ID NO: 27 and SEQ ID NO: 28) appeared as a single band on the SDS-PAGE with molecular weight between about 10 kDa and 15 kDa.

Different from other classes of lectins, when the cell pellet was lysed, similar amounts of the OAA-like lectins comprising SEQ ID NO: 14, SEQ ID NO: 18, SEQ ID NO: 19, and SEQ ID NO: 20 were found in the broth and cell lysate, which indicates its low secretion efficiency and certain challenges in secretion (or binding) of certain lectin proteins.

### Example 16

### Recovery and Purification of Lectins

The instant example describes general methods to recover one or more expressed/produced lectins in substantially pure form from a fermentation broth in which recombinant host cells (e.g., bacterial cells, plant cells, insect cells, and the like) have been fermented. More particularly, such exemplary purification methods including acid treatment by adding sulfuric acid. isoelectric precipitation, ion affinity separation via chromatography column, which separate into two (2) or more fractions, one of which fractions comprises the lectin. The method for each lectin purification is listed below in TABLE 4.

For instance, as shown in TABLE 4, the fermentation crude of certain OAA-like and galectin-1 lectins (SEQ ID NO: 14, SEQ ID NO: 19, SEQ ID NO: 20, SEQ ID NO: 22, SEQ ID NO: 23, SEQ ID NO: 27 and SEQ ID NO: 28), were concentrated and ammonium sulfate added to the final concentration of 1 M. The solution was loaded onto a HIC column pre-equilibrated with 20 mM Tris-HCl (pH7.5) supplemented with 1 M ammonium sulfate. The target lectin protein was eluted through a gradient with 0-1 M ammonium sulfate in 20 mM Tris-HCl (pH7.5) buffer. The fractions containing the lectin protein were concentrated, buffer exchanged and pooled into one (1) fraction. The final concentrated samples of lectins (SEQ ID NO:19, SEQ ID NO: 20, SEQ ID NO: 22, SEQ ID NO: 23. SEQ ID NO: 27, and SEQ ID NO: 28) were formulated with 20 mM Tris, pH 7.5, 150 mM NaCl buffer and 40% w/w glycerol. The final concentrated samples of lectins (SEQ ID NO: 14) were formulated with 20 mM NaPi, pH 7.5, 150 mM NaCl buffer and 40% w/w glycerol. One special case is that the galectin-1 lectins (SEQ ID NO: 27 and SEQ ID NO: 28 are sensitive to low pH (e.g., pH 2.0). These two lectins form precipitation when the pH was adjusted to pH 2.0 by adding 1 mole sulfuric acid (H₂SO₄) dropwise. In other embodiments (TABLE 4) the purification of the lectins (SEQ ID NO: 5, SEQ ID NO: 10, SEQ ID NO:16, SEQ ID NO:17, and SEQ ID NO:25) from a heterologous host were greatly aided by low pH treatment. In the instant case, a pH of about pH 2 was most useful for separating (e.g., background) host proteins from the target lectin protein(s). For example, the host proteins precipitated at pH 2, wherein the host proteins and the target lectin can be separated using centrifuge. The lectins retained in the supernatant were collected and the pH adjusted to neutral by add 2 mole sodium hydroxide and added ammonium sulfate to the final concentration of 1 M. Then, the samples which contain lectins were loaded to HIC column as described above. The final concentrated samples were formulated with 20 mM Tris, pH 7.5, 150 mM NaCl buffer and 40% w/w glycerol.

In certain other embodiments (TABLE 4), the purification of the ricin-like lectin (SEQ ID NO: 24) from a heterologous host was greatly aided by low pH treatment. In the instant case, a pH of about pH 2 was most useful for separating host proteins from the target lectin. The host proteins precipitated at pH 2, wherein the host proteins and the target lectin can be separated using centrifuge. The lectins in the supernatant were collected and the pH was adjusted to neutral by adding 2 mole sodium hydroxides. The final concentrated samples were formulated with 20 mM NaCitrate, pH 3.5, 150 mM NaCl buffer and 40% w/w glycerol.

In other one or more embodiments, (TABLE 4). the purification of the jacalin-like lectin (SEQ ID NO: 8) from a heterologous host were ultrafiltered and added ammonium sulfate to the final concentration of 1 M. The solution was loaded onto a HIC column pre-equilibrated with 20 mM NaAc, pH 5.0 supplemented with 1 M ammonium sulfate. The target lectin protein was eluted with 0 to 1 M ammonium sulfate gradient. The fractions containing target protein were pooled and buffer exchanged into 20 mM Tris-HCl pH7.5, then incubated at 4° C. for overnight to facilitate the isoelectric precipitation. The resulting target protein pellet was resuspended in 20 mM NaCitrate, pH 3.5, 150 mM NaCl buffer and 40% w/w glycerol.

In certain other embodiments (TABLE 4), the purification of the CVN-like lectin (SEQ ID NO: 11) from a heterologous host were ultrafiltered and added ammonium sulfate to the final concentration of 1 M. The solution was loaded onto a HIC column pre-equilibrated with 20 mM Tris, pH 7.5 supplemented with 1 M ammonium sulfate. The target protein was eluted with 0 to 1 M ammonium sulfate gradient. The fractions containing target protein were pooled and buffer exchanged into 20 mM NaAc, pH 5.0, then applied to a AIEX column pre-equilibrated with 20 mM NaAc, pH 5.0. The target protein was eluted with 0 to 0.5 M NaCl gradient. The resulting target protein fractions were then pooled and concentrated via the 5K Amicon Ultra devices, then stored in 20 mM Tris, pH 7.5, 150 mM NaCl buffer and 40% w/w glycerol at −20° C.

In yet other embodiments (TABLE 4), the purification of the OAA-like lectin (SEQ ID NO: 15) from a heterologous host was ultrafiltered and buffer exchanged into 20 mM NaPi, PH6.0, then applied to a AIEX column pre-equilibrated with 20 mM NaPi, PH6.0. The target protein was eluted with 0 to 0.5 M NaCl gradient. The resulting target protein fractions were then pooled and concentrated via the 5K Amicon Ultra devices, then stored in 20 mM Tris, pH 7.5, 150 mM NaCl buffer and 40% w/w glycerol at −20° C.

In certain other embodiments (TABLE 4), the purification of the OAA-like lectin (SEQ ID NO: 21) from a heterologous host were ultrafiltered and ammonium sulfate added to the final concentration of 1 mole. The solution was loaded onto a HIC column pre-equilibrated with 20 mM Tris, pH 7.5 supplemented with 1 M ammonium sulfate. The target protein was eluted with 0 to 1 M ammonium sulfate gradient. The fractions containing target protein were pooled and buffer exchanged into 20 mM NaAc, pH 5.5, then applied to a AIEX column pre-equilibrated with 20 mM NaAc, pH 5.5. The target protein was eluted with 0 to 0.5 M NaCl gradient. The resulting target protein fractions were then pooled, and buffer exchanged into 20 mM NaAc, pH 5.0, then applied to a CIEX column pre-equilibrated with 20 mM NaAc, pH 5.0. The target protein was eluted with 0 to 0.5 M NaCl gradient. The resulting target protein fractions concentrated via the 5K Amicon Ultra devices, then stored in 20 mM NaPi, pH 7.0, 150 mM NaCl buffer and 40% w/w glycerol at −20° C. In addition, the OAA-like lectin (SEQ No 21) was totally precipitated after adjusting the pH to 2 and the protein is still insoluble after adjusting the pH to neutral.

In certain other embodiments (TABLE 4), the purification of the galectin-1 lectin (SEQ ID NO: 26) from a heterologous host were ultrafiltered and exchanged into 20 mM NaPi, pH 7.0. The solution was loaded onto a CIEX column pre-equilibrated with 20 mM NaPi, pH 7.0. The target protein was eluted with 0 to 0.5 M NaCl gradient. The fractions containing target protein were pooled and buffer exchanged into 20 mM NaPi, pH 7.0, then applied to a AIEX column pre-equilibrated with 20 mM NaPi, pH 7.0. The target protein was eluted with 0 to 0.5 M NaCl gradient. The fractions containing target protein were pooled and buffer exchanged into 20 mM Tris, pH 7.5 and add ammonium sulfate to final concentration of 1 mole, then applied to a HIC column pre-equilibrated with 20 mM Tris, pH 7.5 supplemented with 1 M ammonium sulfate. The target protein was eluted with 0 to 1 M ammonium sulfate gradient. The fractions containing target protein were pooled and buffer exchanged into 20 mM Tris. pH 7.5, then applied to a AIEX column pre-equilibrated with 20 mM Tris, pH 7.5. The target protein was eluted with 0 to 0.5 M NaCl gradient. The resulting target protein fractions were then pooled and concentrated via the 5K Amicon Ultra devices, then stored in 20 mM Tris, pH 7.5, 150 mM NaCl buffer and 40% w/w glycerol at −20° C. Similar with galectin-1 like lectin (SEQ No 27 and 28), the galectin-1 lectin (SEQ No 26) is also sensitive to pH 2 and high ratio of the lectin precipitated when the pH of the broth adjusted to 2.

TABLE 4
METHODS FOR LECTIN PURIFICATION

| SEQ ID NO | Method | Purity % | Formulation |
|---|---|---|---|
| 5 | Step 1: Acid treatment | 49 | 20 mM Tris, pH 7.5, 150 mM NaCl |
| | Step 2: HIC | | buffer and 40% w/w glycerol |
| | Step 3: Formulation methods | | |
| 8 | Step 1: HIC | 96 | 20 mM NaCitrate, pH 3.5, 150 mM NaCl |
| | Step 2: Isoelectric Precipitation | | buffer and 40% w/w glycerol. |
| | at pH 7.5 | | |
| | Step 3: Formulation methods | | |
| 10 | Step 1: Acid treatment | 71 | 20 mM Tris, pH 7.5, 150 mM NaCl |
| | Step 2: HIC | | buffer and 40% w/w glycerol |
| | Step 3: Formulation methods | | |
| 11 | Step 1: HIC | 17 | 20 mM Tris, pH 7.5, 150 mM NaCl |
| | Step 2: AIEX | | buffer and 40% w/w glycerol |
| | Step 3: Formulation methods | | |
| 14 | Step 1: HIC | 73 | 20 mM NaPi, pH 7.0, 150 mM NaCl |
| | Step 2: Formulation methods | | buffer and 40% w/w glycerol |
| 15 | Step 1: AIEX | 90 | 20 mM Tris, pH 7.5, 150 mM NaCl |
| | | | buffer and 40% w/w glycerol |
| 16 | Step 1: Acid treatment | 98 | 20 mM Tris, pH 7.5, 150 mM NaCl |
| | Step 2: HIC | | buffer and 40% w/w glycerol |
| | Step 3: Formulation | | |
| 17 | Step 1: Acid treatment | 70 | 20 mM Tris, pH 7.5, 150 mM NaCl |
| | Step 2: HIC | | buffer and 40% w/w glycerol |
| | Step 3: Formulation methods | | |
| 19 | Step 1: HIC | 60 | 20 mM Tris, pH 7.5, 150 mM NaCl |
| | Step 2: Formulation | | buffer and 40% w/w glycerol |
| 20 | Step 1: HIC | 85 | 20 mM Tris, pH 7.5, 150 mM NaCl |
| | Step 2: Formulation | | buffer and 40% w/w glycerol |
| 21 | Step 1: HIC | 80 | 20 mM NaPi, pH 7.0, 150 mM NaCl |
| | Step 2: AIEX | | buffer and 40% w/w glycerol |
| | Step 3: CIEX | | |
| | Step 4: Formulation | | |
| 22 | Step 1: HIC | 94 | 20 mM Tris, pH 7.5, 150 mM NaCl |
| | Step 2: Formulation | | buffer and 40% w/w glycerol |
| 23 | Step 1: HIC | 100 | 20 mM Tris, pH 7.5, 150 mM NaCl |
| | Step 2: Formulation | | buffer and 40% w/w glycerol |
| 24 | Step 1: Acid treatment | 86 | 20 mM NaCitrate, pH 3.5, 150 mM NaCl |
| | Step 2: Formulation | | buffer and 40% w/w glycerol |
| 25 | Step 1: Acid treatment | 98 | 20 mM Tris, pH 7.5, 150 mM NaCl |
| | Step 2: HIC | | buffer and 40% w/w glycerol. |
| | Step 3: Formulation | | |
| 26 | Step 1: CIEX | 80 | 20 mM Tris, pH 7.5, 150 mM NaCl |
| | Step 2: AIEX | | buffer and 40% w/w glycerol |
| | Step 3: HIC | | |
| | Step 4: AIEX | | |
| | Step 5: Formulation | | |
| 27 | Step 1: HIC | 50 | 20 mM Tris, pH 7.5, 150 mM NaCl |
| | Step 2: Formulation | | buffer and 40% w/w glycerol. |
| 28 | Step 1: HIC | 50 | 20 mM Tris, pH 7.5, 150 mM NaCl |
| | Step 2: Formulation | | buffer and 40% w/w glycerol. |

Columns used for lectins purification which were listed in TABLE 4 are HIC: Column HiPrepTM Phenyl FF (high sub) 16/10; AIEX: Column HiPrepTM Q FF 16/10; CIEX: Column HiPrepTM SP FF 16/10

### Example 17

### Assaying Hemagglutination Activity of Lectins

The instant example describes methods to screen the binding activity of the of one or more purified lectins described above using a hemagglutination assay. The principle is that active lectins react with specific carbohydrate moieties on red blood cell surfaces, resulting in the formation of a diffuse matrix, while non-active lectins cannot bind red blood cells resulting in the formation of noticeable clumps. This allows for a clear visual distinction between active and non-active lectin samples. More particularly, lectin hemagglutination capability (activity) was evaluated using erythrocytes from fifteen (15) different animal sources (i.e., dog, rabbit, guinea pig, mouse, rat, human, chicken, turkey, duck, goose, pig, bovine, horse, sheep, and goat) and processed according to the Materials and Methods described herein.

### Materials

Materials included the following: (i) Animal erythrocytes in Alsever's solution (Sbjbio company, Nanjing. China), including dog, rabbit, guinea pig, mouse, rat, human, chicken, turkey, duck, goose, pig, bovine, horse, sheep, and goat erythrocytes, (ii) 200 mM phosphate buffer (PB. pH 7.4), and (iii) 96-well round bottom microwell plate (Nunc, Thermo Scientific, USA).

### Methods

(i) Hemagglutination test was used to detect lectins in both purified and crude samples. Purified lectin samples were diluted to an initial concentration of 200-400 g/ml using PB buffer, then dispensed 50 μL of each to the sample well of a 96-well round bottom microplate. Crude samples were diluted by estimating the protein expression level based on the SDS-PAGE gel. (ii) 50 μl of host strain supernatant was dispensed to the control wells of the above microplate and used as the negative controls. A column of 50 μL PB buffer was also included in the above microplate to determine the possible buffer effect. (iii) Animal erythrocytes were diluted to an initial concentration of 2% (v/v) using PB buffer, and then added 50 μl of each to the above samples. (iv) The mixture was pipetted for 30 seconds to combine, then allow the plate to settle at room temperature for 60 minutes. (v) The hemagglutination activity was determined by visual examination. Active lectins were characterized by the formation of a diffuse network, whereas non-active lectins were observed to form a sediment button at the bottom of the well.

### Results

As shown in FIG. 12, the hemagglutination capability of lectin samples could be easily determined by visually distinguishing the even suspension with no signs of clumping wells for positive samples and a sediment button at the bottom of the wells for negative samples. PB buffer and host strain supernatant showed no hemagglutination capability (FIG. 12A and FIG. 12B). A dose-response pattern of hemagglutination activity for active samples was detected when using different doses of lectins on 1% mouse erythrocytes showing lectin hemagglutination capability was dose-dependent (FIG. 12A). This shows the hemagglutination assay is a feasible method to differentiate strong positive (e.g., SEQ ID NO: 1 at 200 μg/ml in FIG. 12A), weak positive (e.g., SEQ ID NO: 5 at 0.78 μg/ml in FIG. 12A), intermediate positive (e.g., SEQ ID NO: 1 at 12.5 μg/ml in FIG. 12A), and negative samples (e.g., SEQ ID NO: 8 in FIG. 12A). At least two parallel plates were made for each test to ensure data accuracy and the results showed good reproducibility (FIG. 12B). The lectin hemagglutination was stable for about 1-2 hours at room temperature and the observations and interpretations can vary from test to test, batch of animal erythrocytes, type and concentration of lectins, incubation time and temperature used in the assay. Therefore, in the present example, the hemagglutination assay was not intended to be used as a quantitative method, but rather to detect positive samples. The most obvious positive results were recorded in FIG. 13 and the corresponding effective animal erythrocytes are summarized below in TABLE 5.

TABLE 5
HEMAGGLUTINATION ACTIVITY OF LECTINS PRODUCED

| | | Concentration | Hemagglutination |
|---|---|---|---|
| SEQ ID | Sample | (μg/ml) | capability |
| 1 | Purified | 200 | Dog, mouse, rat |
| 2 | Purified | 200 | Mouse, rat |
| 5 | Purified | 200 | Mouse, rat, bovine, horse, goat |
| 6 | Purified | 200 | Mouse, rat |
| 7 | Crude | — | — |
| 8 | Purified | 200 | Dog, rabbit |
| 10 | Purified | 50 | Dog, rabbit, guinea pig, mouse, rat |
| 11 | Purified | 200 | — |
| 12 | Purified | 0.5-400 | — |
| 14 | Purified | 200 | — |
| 15 | Purified | 200 | Dog, guinea pig, mouse, rat, human, |
| | | | duck, goose, horse, goat |
| 16 | Purified | 50 | Guinea pig, mouse, rat |
| 17 | Purified | 200 | — |
| 18 | — | — | ND |
| 19 | Purified | 0.5-400 | — |
| 20 | Purified | 400 | Rabbit |
| 21 | Purified | 5 | Guinea pig, mouse, rat |
| 22 | Purified | 5 | Guinea pig, mouse, rat, pig, bovine, |
| | | | horse, goat |
| 23 | Purified | 50 | Guinea pig, mouse, rat |
| 24 | Crude | — | Human, chicken, turkey, duck, goose, |
| | | | pig, bovine, horse, sheep, goat |
| 25 | Purified | 0.5-400 | — |
| 26 | Crude | — | Dog, rabbit, guinea pig, mouse, rat, |
| | | | human, chicken, turkey, duck, goose, |
| | | | pig, bovine, horse, sheep, goat |
| 27 | Purified | 0.5-400 | — |
| 28 | Purified | 50 | Guinea pig, mouse, rat, pig, bovine, |
| | | | horse, goat |
| 29 | Crude | — | Rabbit, guinea pig, mouse, rat, pig, |
| | | | bovine, horse, goat |
| 30 | Crude | — | Mouse, rat |
| 31 | Crude | — | Mouse, rat |
| 32 | Crude | — | Rabbit, mouse, rat, pig, bovine, |
| | | | horse, goat |
| 33 | Crude | — | Rabbit, guinea pig, mouse, rat, pig, |
| | | | bovine, horse, goat |
| 34 | Crude | — | — |
| 35 | Crude | — | Mouse, rat |
| 36 | Crude | — | Mouse, rat |
| 37 | Crude | — | Mouse, rat |
| 38 | Crude | — | Rabbit, guinea pig, mouse, rat |
| 39 | Crude | — | — |

ND—not detected yet.

### REFERENCES

PCT Publication No. WO2002/14490
PCT Publication No. WO2003/083125
PCT Publication No. WO2005/118627
PCT Publication No. WO2007/064844
PCT Publication No. WO2008/022303
PCT Publication No. WO2010/014248
PCT Publication No. WO2014/197650
PCT Publication No. WO2016/130628
PCT Publication No. WO2019/108656
US Patent Publication No. US20020127675
US Patent Publication No. US20040204365
US Patent Publication No. US20110189105
US Patent Publication No. US20110263485
Ausubel et al., “Current Protocols in Molecular Biology, published by Greene Publishing Assoc. and Wiley-Interscience (1987).
Ayouba et al., “Legume lectins interact with muramic acid and N-acetylmuramic acid”, FEBS. 289(1): 12-104, 1991.
Cole et al., “Differentiation of Bacillus anthracis and Other Bacillus Species by Lectins”, J. Clinical Microbiol., 19(1): 48-54, 1984.
El-Araby et al., “Characterization and antimicrobial activity of lectins purified from three Egyptian leguminous seeds”, AMB Express, 10:90 (pages 1-14), 2020.
Gengenbach et al., “Comparison of microbial and transient expression (tobacco plants and plant-cell packs) for the production and purification of the anticancer mistletoe lectin viscumin”, Biotechnology and Bioengineering, 116, pages 2236-2249, 2019.
Hirayama et al., “High-Mannose Specific Lectin and Its Recombinants from a Carrageenophyta Kappaphycus alvarezii Represent a Potent Anti-HIV Activity Through High-Affinity Binding to the Viral Envelope Glycoprotein gp120”, Mar. Biotechnol., Vol 18, pages 144-160, 2016.
Lagarda-Diaz et al., “Legume Lectins: Proteins with Diverse Applications”, Int. J. Mol. Sci., 18, 1242, 2017.
O'Keefe et al., “Scaleable manufacture of HIV-1 entry inhibitor griffithsin and validation of its safety and efficacy as a topical microbicide component”, PNAS, Vol. 106, No. 15, pages 6099-6104, 2009.
Petrova et al., “Engineering Lactobacillus rhamnosus GG and GR-1 to express HIV-inhibiting griffithsin”, International Journal of Antimicrobial Agents, volume 52, issue 5, pages 599-607, 2018.
Petrova et al., “The lectin-like protein 1 in Lactobacillus rhamnosus GR-1 mediates tissue-specific adherence to vaginal epithelium and inhibits urogenital pathogens”, Scientific Reports 6, Article No. 37437, 2016.
Peumans and Van Damme, “Plant Lectins: Versatile Proteins with Important Perspectives in Biotechnology”, Biotechnology and Genetic Engineering Reviews, 15:1, 199-228, 1998.
Pevzner et al., “Differences in amino acid sequences of mistletoe lectin I and III B-subunits determining carbohydrate binding specificity”, Biochimica et Biophysica Acta 1675:155-164, 2004.
Sambrook et al., “Molecular Cloning: A Laboratory Manual” Cold Spring Harbor Laboratory: Cold Spring Harbor, N.Y. (1989), (2001) and (2012).
Singh and Sarathi, “Insights of Lectins—A review”, International J. Scientific & Engineering Res., Volume 3, Issue 4, 2012.
Whitley et al., “Burkholderia oklahomensis agglutinin is a canonical two-domain OAA-family lectin: structures, carbohydrate binding, and anti-HIV activity”, FEBS J., 280(9): 2056-2067, 2013.

1. A method for intracellular production of a lectin in a Bacillus sp. cell comprising:
introducing into the cell an expression cassette encoding a lectin, wherein the cassette comprises an upstream promoter operably linked to a downstream open reading frame (ORF) encoding the lectin, and
fermenting the cell for the production of the lectin.

2. (canceled)

3. A method for extracellular production of a lectin in a Bacillus sp. cell comprising:
introducing into the cell an expression cassette encoding a lectin, wherein the cassette comprises an upstream promoter operably linked to a downstream nucleic acid encoding a signal sequence operably linked to a downstream open reading frame (ORF) encoding the lectin, and
fermenting the cell for the production of the lectin.

4. (canceled)

5. A method for combined intracellular and extracellular production of a lectin in a Gram-positive bacterial cell comprising:
introducing into the cell a first and a second expression cassette encoding a lectin, wherein the first cassette comprises an upstream promoter operably linked to a downstream open reading frame (ORF) encoding the lectin and the second cassette comprises an upstream promoter sequence operably linked to a downstream nucleic acid encoding a signal sequence operably linked to a downstream ORF encoding the lectin, and
fermenting the cell for the production of the lectin.

6. (canceled)

7. A method for recovering an intracellular lectin produced by a Bacillus sp. cell comprising:
constructing and fermenting a Bacillus sp. cell according to claim 1,
lysing cells at end of the fermentation to obtain a lysed cell broth, heat treating the lysed broth at pH between 1.5 and 8.5, then cooling broth and harvesting the cooled broth,
subjecting the harvested cooled broth to a clarification process, and
subjecting the clarified broth to a concentration process,
wherein the lectin is recovered in the clarified concentrated broth.

8. A method for recovering a secreted lectin produced by a Bacillus sp. cell comprising:
constructing and fermenting a Bacillus sp. cell according to claim 2, and harvesting the broth,
subjecting the harvested broth to a clarification process, and
subjecting the clarified broth to a concentration process,
wherein the lectin is recovered in the clarified concentrated broth.

9-13. (canceled)

14. A method for producing and recovering a high purity griffithsin (GRFT) protein preparation comprising:
constructing and fermenting a recombinant Bacillus sp. cell expressing a heterologous GRFT protein,
(b) lysing cells at end of the fermentation to obtain a lysed cell broth, and treating the lysed broth by holding broth for about 1 to about 4 hours at a pH of about 4.8 to about 5.2 and a temperature of about 50° C. to about 80° C.,
clarifying the broth by a filtration or microfiltration process, and concentrating the clarified broth by an ultrafiltration process,
performing a crystallization process on the concentrated broth, the crystallization process comprising adding about 2% sodium sulfate to the concentrate, adjusting pH to about 3 and incubating at about 22° C. for a sufficient amount of time,
centrifuging the incubated concentrate, decanting supernatant to harvest the GRFT crystal, dissolving the GRFT crystal in about 100 mM sodium acetate buffer at about pH 4.5 to about 5.5 to obtain an aqueous GRFT protein preparation, and centrifuging the GRFT preparation to remove any insoluble impurities therein and collecting the supernatant or filtering the GRFT preparation to remove any insoluble impurities therein and collecting the filtrate,
wherein the supernatant collected, or filtrate collected, comprises a high purity GRFT protein preparation.

15. The method of claim 14, wherein the high purity GRFT preparation comprises a native GRFT protein, or a variant GRFT protein.

16. The method of claim 14, wherein the high purity GRFT preparation is at least 2.0 times higher in purity than the recovered GRFT concentrate, as determined via the GRFT concentration measured at A₂₈₀ nm, and/or wherein the GRFT is the major band, or the only band of about 12.7 kDa in the high purity GRFT preparation when visualized by SDS-PAGE.

17-22. (canceled)
